(19) United States
(12) Patent Application Publication
(10) Pub. No.: US 2021/0335447 A1
(43) Pub. Date: Oct. 28, 2021

(54) METHODS AND SYSTEMS FOR ANALYSIS OF RECEPTOR INTERACTION

(71) Applicant: REGENERON PHARMACEUTICALS, INC., Tarrytown, NY (US)

(72) Inventors: Wen Zhang, Tarrytown, NY (US); Jing He, Tarrytown, NY (US); Namita Gupta, Tarrytown, NY (US); Gurinder S. Atwal, Tarrytown, NY (US); Peter Hawkins, Tarrytown, NY (US)

(21) Appl. No.: 17/236,998

(22) Filed: Apr. 21, 2021

Related U.S. Application Data
(60) Provisional application No. 63/111,395, filed on Nov. 9, 2020.
(60) Provisional application No. 63/090,498, filed on Oct. 12, 2020.
(60) Provisional application No. 63/013,480, filed on Apr. 21, 2020.

(51) Int. Cl. G16B 5/20; G16B 15/30; G16B 40/00; G06N 20/00; G06N 5/04

(52) U.S. Cl. CPC G16B 5/20; G16B 15/30; G06N 5/04; G06N 20/00; G16B 40/00

(57) ABSTRACT

A computational framework for high-throughput mapping, validating, and predicting receptor sequence interactions is described.

### CROSS REFERENCE TO RELATED PATENT APPLICATION

This application claims priority to U.S. Provisional Application No. 63/111,395, filed Nov. 9, 2020, U.S. Provisional Application No. 63/090,498, filed Oct. 12, 2020, and U.S. Provisional Application No. 63/013,480, filed Apr. 21, 2020, which are herein incorporated by reference in their entirety.

### BACKGROUND

T cell antigen specificity, mediated via T cell receptors (TCRs), is a hallmark of cellular immunity. TCRs are heterodimeric proteins found on the T cell surface, commonly comprised of an α- and β-chain. The TCR α- and β-chain genes are composed of discrete V, D (β-chain only) and J segments that are joined by somatic recombination during T cell development. This genetic rearrangement generates a highly diverse TCR repertoire (estimated to range from 1015 to 1061 possible receptors in human) to ensure efficient control of viral infections and other pathogen-induced diseases. TCR diversity is primarily exhibited in complementarity determining region (CDR) loops (CDR1, CDR2 and CDR3), which engage peptides that are presented by major histocompatibility complex (MHC) proteins, and therefore directly determines the specificity of T cell pMHC binding.

Although the factors underlying TCR-pMHC recognition are not fully understood, recent studies have shown that T cells binding to a particular pMHC share common TCR sequence features and, in select cases, it is possible to predict the specific binding probability of an unseen TCR sequence based on learned TCR sequence features. However, these studies were limited by the quantity and diversity of training data generated by traditional single multimer sorting or antigen re-exposure assays. Further understanding of TCR-pMHC specific binding requires innovation in both computational and experimental methods. 10× Genomics recently published a dataset generated from their highly multiplexed pooled dextramer binding immune profiling platform that couples feature-barcoded dextramers and single cell TCR sequencing. This approach makes it feasible to generate high-dimensional pMHC specific binding data at the single cell level with paired T cell α- and β-chain sequences, whereas other large-scale pooled multimer approaches only estimate the composition of pMHC specific binding T cells.

As with any other high throughput technology, highly multiplexed dextramer binding data are often associated with low signal-to-noise ratios. This makes it bioinformatically challenging to reliably identify TCR-pMHC binding events using such large-scale binding datasets. Unexpectedly high cross-HLA and cross-pMHC associations were observed from the binding events that 10× Genomics provided (FIG. 11A). This low signal-to-noise dataset calls for more sophisticated computational normalization methods to discriminate true TCR-pMHC binding events from non-specific background.

As next-generation screening technologies have increased the volume of available TCR-pMHC binding data, state-of-the-art functional classifiers to computationally validate and subsequently predict TCR-pMHC specific recognition have become more feasible. While the results from initial TCR-pMHC binding classifiers are encouraging, they were only trained using CDR loop sequences and thus unable to learn the overall complex sequence patterns from full-length TCR sequences, resulting in sub-optimal prediction accuracy for highly diverse pMHC binding TCRs. Leveraging the ability of deep learning algorithms to learn complex patterns, several deep learning frameworks were recently proposed to uncover binding patterns in large, highly complex TCR sequence datasets.

In this study, a computational framework for mapping, computationally validating, and predicting TCR-pMHC specific recognition using highly multiplexed dextramer binding data is described.

### BRIEF SUMMARY

Disclosed are methods comprising receiving single cell sequencing data comprising single cell sequence data, dextramer sequence data, and single cell T-Cell Receptor (TCR) sequence data; filtering, from the dextramer sequence data, based on the single cell sequence data, data associated with low-quality cells; adjusting, based on a measure of background noise, the dextramer sequence data; filtering, from the dextramer sequence data, based on the single cell TCR-data, data according to a presence or an absence of an α-chain or a β-chain; and identifying data remaining in the normalized filtered dextramer sequence data as associated with reliable TCR-pMHC binding events.

Disclosed are methods comprising receiving single cell sequence data, dextramer sequence data, and single cell T Cell Receptor (TCR) sequence data; determining, for each cell represented in the dextramer sequence data, based on the single cell sequence data, a number of genes; removing, from the dextramer sequence data, data associated with cells having a number of genes outside of a gene threshold range; determining, for each cell represented in the dextramer sequence data, based on the single cell sequence data, a fraction of mitochondrial gene expression; removing, from the dextramer sequence data, data associated with cells having a fraction of mitochondrial gene expression that exceeds a gene expression threshold; determining, based on the dextramer sequence data, sorted dextramer sequence data wherein the sorted dextramer sequence data comprises sorted test dextramer sequence data and negative control dextramer sequence data and unsorted dextramer sequence data, wherein the unsorted dextramer sequence data comprises unsorted test dextramer sequence data; determining, for each cell represented in the dextramer sequence data, based on the negative control dextramer sequence data, a maximum negative control dextramer signal; determining, for each cell represented in the dextramer sequence data, based on the sorted test dextramer sequence data, a maximum sorted dextramer signal; determining, for each cell represented in the dextramer sequence data, based on the unsorted test dextramer sequence data, a maximum unsorted dextramer signal; estimating, based on the maximum negative control dextramer signals, a dextramer binding background noise; estimating, based on the maximum sorted dextramer signals and the maximum unsorted dextramer signals, a dextramer sorting gate efficiency; determining, based on the dextramer binding background noise and the dextramer sorting gate efficiency, a measure of background noise; subtracting, for each cell represented in the dextramer sequence data, the measure of background noise from a dextramer signal associated with each cell; performing, for each cell represented in the dextramer sequence data, cell-wise normalization on the dextramer signals associated with each cell; performing, for each cell represented in the dextramer sequence data, pMHC-wise normalization; determining, for each cell represented in the dextramer sequence data, based on the single cell TCR sequence data, a presence or an absence of at least one α-chain and at least one β-chain; removing, from the normalized dextramer sequence data, based on the presence or the absence of the at least one α-chain and the at least one β-chain, data associated with cells having only an α-chain, only a β-chain, or multiple α- or β-chains; and identifying data remaining in the normalized dextramer sequence data as associated with reliable TCR-pMHC binding events.

Disclosed are methods comprising performing TCR-pMHC binding specificity data normalization on dextramer sequence data to identify a plurality of TCR-pMHC binding events; determining, based on the normalized dextramer sequence data, a training dataset comprising a plurality of TCR sequences wherein each TCR sequence is associated with a binding affinity; determining, based on the plurality of TCR sequences, a plurality of features for a predictive model; training, based on a first portion of the training dataset, the predictive model according to the plurality of features; testing, based on a second portion of the training dataset, the predictive model; and outputting, based on the testing, the predictive model.

Disclosed are methods comprising presenting, to a trained predictive model, an unknown TCR sequence, wherein the trained predictive model is trained based on a training data set derived according to the disclosed methods; and predicting, by the trained predictive model, a binding affinity.

Disclosed are methods comprising receiving single cell sequence data, dextramer sequence data, and single cell T Cell Receptor (TCR) sequence data, determining, for each cell represented in the dextramer sequence data, based on the single cell sequence data, a number of genes, removing, from the dextramer sequence data, data associated with cells having a number of genes outside of a gene threshold range, determining, for each cell represented in the dextramer sequence data, based on the single cell sequence data, a fraction of mitochondrial gene expression, removing, from the dextramer sequence data, data associated with cells having a fraction of mitochondrial gene expression that exceeds a gene expression threshold, determining, based on the dextramer sequence data, sorted dextramer sequence data wherein the sorted dextramer sequence data comprises sorted test dextramer sequence data and negative control dextramer sequence data, determining, for each cell represented in the dextramer sequence data, based on the negative control dextramer sequence data, a maximum negative control dextramer signal, determining, for each cell represented in the dextramer sequence data, based on the sorted test dextramer sequence data, a maximum sorted dextramer signal, estimating, based on the maximum negative control dextramer signals and the maximum sorted dextramer signals, a dextramer binding background noise, determining, for each cell represented in the dextramer sequence data, based on the single cell TCR sequence data, a presence or an absence of at least one α-chain and at least one β-chain, removing, from the dextramer sequence data, based on the presence or the absence of the at least one α-chain and the at least one β-chain, data associated with cells having only an α-chain, only a β-chain, or multiple α- or β-chain, determining, for each dextramer binding to a given cell represented in the dextramer sequence data, a ratio of dextramer signal within the cell to a sum of all dextramers binding to the cell (a measure of the dextramer binding specificity to the cell), determining, for each dextramer binding to a given TCR clonotype of each cell represented in the dextramer sequence data, a fraction of T cells within a clone binding to a particular dextramer (a measure of the dextramer binding specificity to the clonotype to which the cell belongs), determining, for each dextramer binding to a given cell represented in the dextramer sequence data, based on the measure the of the dextramer binding specificity to the cell and the measure of the dextramer binding specificity to the clonotype to which the cell belongs, a corrected dextramer signal associated with each dextramer binding to the cell, performing, for each cell represented in the dextramer sequence data, cell-wise normalization on the dextramer signals associated with each cell, performing, for each cell represented in the dextramer sequence data, pMHC-wise normalization, and identifying, based on a threshold, data remaining in the normalized dextramer sequence data as associated with reliable TCR-pMHC binding events.

Disclosed are apparatus configured to perform any of the disclosed methods.

Disclosed are computer readable mediums having processor-executable instructions embodiment thereon configured to cause an apparatus to perform any of the disclosed methods.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows an example operational environment.

FIG. 2 shows an experimental approach for generating multi-omics high-throughput TCR-pMHC binding data: PBMC T cells from healthy human donors were labeled for sorting on CD8+ cells. Sorted CD8+ T cells were stained with a pool of 50 dCODE Dextramer antibodies. Dextramer positive CD8+ T cells were sorted by flow cytometry and were captured individually as input for the 10× Genomics single cell sequencing library preparation. Three libraries were generated for gene expression, cell surface protein/dCODE expression, paired TCR sequences for each CD8+ T cell.

FIG. 3 shows an example method.

FIG. 4 shows an example method.

FIG. 5 shows an example method.

FIGS. 6A and B show an example of the ICON (Integrative COntext-specific Normalization) workflow schema. a. From the top left to bottom left: I. Distributions of dCODE dextramer raw expression in UMI (Unique Molecular Identifier). The maximum dCODE dextramer expression in UMI in each CD8+ cells from Dex_sorted (maximum UMI of testing dextramers from dextramer sorted CD8+ T cells), NC_dex (maximum UMI of negative control dextramers from dextramer sorted CD8+ T cells) and Dex_unsorted (maximum UMI of testing dextramers in dextramer stained but not sorted control CD8+ cells). II. Filtering out low quality cells based on single cell RNA-seq. Each dot is a T cell. Dots in red are unhealthy cells. III. Estimating dextramer binding background noise (P_{99.9}) and dextramer sorting gate efficiency (argmax D_{s, u}) based on dCODE dextramer expression data. IIII. Adjusting background noise by subtracting Max(P_{99.9}, argmax D_{s, u}). V. Cell and pMHC wise normalization of background subtracted dextramer expression. VI. Selecting cells with single paired TCR αβ chains. VII. Distributions of normalized dextramer expression. UMI*: normalized UMI. Please see the Methods for details. b. TCR-pMHC binding specificities of expanded TCR clonotypes. The 50 largest TCR clones from donor 1 are plotted along with their binding specificities and concordance. A circle indicates that at least one member of the clonotype was classified as specific for a particular pMHC. Circle size indicates the total within-donor clonotype size. Circle color indicates the proportion of cells within the clonotype that bind the Dextramer (the ‘binding concordance’). The left panel: the 50 largest clonotypes that 10× Genomics identified using global cutoffs. The right panel: the 50 largest clonotypes from the pMHC repertoires that contain the 10× Genomics 50 largest clonotypes of donor 1.

FIGS. 7A-7E show the pMHC binding landscape of 10× Genomics dextramer binding data. a. The network of identified pMHC specific binding T cell repertoires. Each node represents an pMHC repertoire and a pie chart of the number of unique paired TCRs from each donor that bind to that pMHC. Donor 1 is gray, Donor 2 is red, and Donor 4 is yellow. The node size denotes the total number of T cells that bind to that pMHC. Each edge represents unique TCR(s) shared by two pMHCs. The thickness of edge represents the number of shared unique TCR(s). b. Majority of identified binders interact with the seven pMHCs. c. The Venn diagram of unique paired binding TCRs identified from donor 1, donor 2 and donor 3. d. Composition of unique paired TCR αβ chains. By TCRB, 1 to 1 means 1 unique TCR β-chain paired with 1 unique TCR α-chain; 1 to >=2 & binding to identical pMHC means unique paired TCRs with shared β-chain but different α-chains recognize the same pMHC; 1 to >=2 & binding to >=2 pMHCs means unique paired TCRs with shared β-chain but different α-chains recognize different pMHCs. By TCRA, 1 to 1 means 1 unique TCR α-chain paired with 1 unique TCR β-chain; 1 to >=2 & binding to identical pMHC means unique paired TCRs with shared α-chain but different β-chains recognize the same pMHC; 1 to >=2 & binding to >=2 pMHCs means unique paired TCRs with shared α-chain but different β-chains recognize different pMHCs. e. TCR-pMHC binding specificity and TCR cross-HLA recognition. Left, a pie chart of T cells binding to one pMHC or to at least 2 pMHCs. Right, a pie chart of T cells: HLA type match binding, supertype match binding or cross-type binding.

FIGS. 8A-8D show a convolutional neural network (CNN) based classification of TCR-pMHC binding TCRs. a. CNN-based TCR sequence classification framework. The left panel, the V and J segments (from alpha and beta) were transformed into embedding vectors. Trainable embeddings were used for the amino acids that constitute CDR3 alpha or beta sequences, and a 1-dimensional CNN was applied to the embedding. Then, all embeddings were concatenate together and fed through connected layers. A SoftMax layer then was used to output the sequence class probability. The right panels, a toy example illustrates the input and out of Deep Learning sequence classifier. Please see the Methods session for details. b. ROC curves for the CNN-based classifier with binomial mode using the 11 curated paired TCR pMHC binding repertoires. Binders are unique TCRs bind to a particular pMHC, and non-binders are unique TCRs bind to other 10 pMHCs. Paired α & β TCR sequences were used as input data. c. Classification power comparison between the CNN-based and the distance-based binary classifiers with the same definition for binders and non-binders as descripted in b. Paired α & β TCR sequences were used as input data (Methods). d. Correlation of pMHC repertoire diversity measured by Shannon entropy and prediction performance between the CNN-based and the distance-based classifiers. ΔAUC=AUC of the CNN-based—AUC of the distance-based.

FIG. 9A-4E shows a CNN-based classification of the top seven pMHC binding repertoires identified from 10× Genomics dataset. a. ROC curves for the CNN-based classifier in binomial mode using the 7 pMHC binding repertoires identified from 10× Genomics high-throughput dataset. Binders are unique TCRs bind to a particular pMHC, and non-binders are unique TCRs bind to other 6 pMHCs. Paired α & β TCR sequences were used as input data. b. ROC curves of the prediction results from the CNN-based classifier using independent testing datasets from VDJdb: T cells binding to A*02:01_GILGFVFTL_Flu-MP_Influenza, A*02:01_ELAGIGILTV_MART-1_Cancer, A*02:01_GLCTLVAML_BMLF1_EBV and A*11:01_AVFDRKSDAK_EBNA-3B_EBV and another set of MART-1 (REGN_A*02:01 ELAGIGILTV_MART-1_Cancer) binders from an in-house independent experiment (Methods). The module was trained by pMHC repertoires identified from 10× Genomics data for the prediction. c. Classification performance comparisons using TCRα only, TCRβ only or paired TCRα & β chains as the sequence input. d. T cell V and J gene segment usages for T cells binding to these seven pMHCs. The gene segments with less than 5% were combined and indicated in grey. e. CDR3 motifs of the 10 most predictive paired TCRs from the 7 pMHC repertoires.

FIGS. 10A-10E show immune phenotypes of pMHC binding CD8+ T cells. a. Classification of pMHC binding cells. Clusters were visualized by UMAP and cell types were represented by different colors. b. The heatmap of the gene or protein expression of cell type marker genes for annotating CD8+ T cell subgroups. C. pMHC binding landscape by T cell immune subtypes. Bars indicate the number of pMHC binding T cells in log 2 scale. d. Expanded clonotypes are enriched in the non-naïve compartment. Each dot represents a unique TCR clone. e. Proportion of HLA match and mismatch binding in naïve and non-naïve binding T cells. Tpm: peripheral memory cells; Tcm: central memory cells; Tem: effector memory cells; Temra: terminally differentiated effector memory cells; Others: other memory cells with marker expression CD43^{lo}KLRG1^{hi} CD127.

FIGS. 11A-11B shows TCR-pMHC binding specificities of expanded clonotypes from the binding events that 10× Genomics identified from each donor. The 50 largest clonotypes are plotted along with their binding specificities and concordance. a. A circle indicates that at least one member of the clonotype was classified as specific for a particular pMHC. Circle size indicates the total within-donor clonotype size. Circle color indicates the proportion of cells within the clonotype that bind the Dextramer (the ‘binding concordance’). b. Scatter plots of cell sorting results for reassessment of CD8+ T cell dextramer binding of 10× Genomics donors 3 and 4 (Methods).

FIGS. 12A-12F are examples of estimating the background of 10× Genomics high-throughput data and adjusting dextramer binding signal. Dex_sorted (maximum UMI of testing dextramers from dextramer sorted CD8+ T cells), NC_dex (maximum UMI of negative control dextramers from dextramer sorted CD8+ T cells) and Dex_unsorted (maximum UMI of testing dextramer in dextramer stained but not sorted control CD8+ cells). a. Scatter plots of number of detected genes versus percentage of mitochondria gene expression using single cell RNA data. Each dot represents a cell. Dots in red are dead cells or doublets. b. The distributions of dextramer expression data before and after ICON process. C & d. Estimating dextramer sorting efficiency. c. accumulated distribution of dextramer UMI. Each dot is a data point of unique dextramer UMIs. d. p-value distribution of the KS test (Dex_sorted vs. Dex_unsorted) using one dextramer UMI data point as a sliding window. The grey dash line is the threshold for dextramer sorting efficiency. e. Scatter plot of Dex_sorted before (x axis) and after (y axis) background subtraction for each donor. f. E_{c}′ density distribution. E_{c}′: the Log-Rank of each dextramer signal within a cell (Methods). Blue dash line is for the threshold of pMHC specific binding.

FIGS. 13A-13C show binding specificities of expanded clonotypes identified by this study from three donors. The 50 largest T cell clones are plotted along with their binding specificities and concordance. Circle size indicates the T cell clone size. Circle color indicates the proportion of cells within the clone that bind the Dextramer, the binding concordance.

FIGS. 14A and 14B show ROC curves for the distance-based classifier using the curated pMHC binding repertoires. b. Shannon entropy scores for the curated pMHC binding repertoires.

FIGS. 15A-15C show a characterization of the top 7 pMHC binding T cell repertoires. a. A pie chart of proportion of HLA type matched, supertype matched and mismatched binding T cells. b. Power law distributions of unique T cell clone sizes of the top 7 pMHC binding repertoires. Lowess Smoothing was used for fitting. c. Simpson's diversity index and TCRB generation probabilities of TCR-pMHC repertoires. R package vegan was used for calculating Simpson's diversity index. TCRB CDR3 amino acid sequence generation probabilities of binders specific to each pMHC was calculated using OLGA. Then, the fraction of the repertoire specific (represented by red triangles) to each pMHC is obtained as the sum of the generation probabilities for each of the corresponding CDR3 sequences as Sethna et al. described. The result shows that the net fraction of TCRs specific to these pMHCs is large (range from 10⁷ to 10⁴) in) the sense defined by the inverse of the number of independent TCR recombination events (10⁸), meaning that any individual is likely to have these binding T cells in their T repertoire. Each dot in the TCRB generation probability figure represent a unique T cell clone and the color bar indicate the T cell clone size.

FIGS. 16A-16C show a classification of TCR-pMHC binding TCRs. a. Distance-distance distributions of pMHC binders and non-binders using α-chain only, β-chain only and paired αβ chains. b. ROC curves for the distance-based classifier using the top 7 pMHC binding repertoires identified from 10× Genomics high-throughput dataset. Paired α & β TCR sequences were used as input data. c. Comparison of the classification power of the CNN-based and the distance-based classifiers.

FIGS. 17A and 17B show CDR3 motifs of the four pMHC binding repertoires from the overlap of VDJdb and the top 7 pMHC repertoires identified from 10× Genomics high-throughput data. b. ROC curves for the CNN-based classifier in multinomial mode using the 7 pMHC binding repertoires identified from 10× Genomics high-throughput dataset. Paired α & β TCR sequences were used as input data.

FIGS. 18A and 18B show an example of cluster of pMHC binding CD8+ cells using single cell RNA-seq data. a. by number of clusters. b. Overlaid with donor information.

FIG. 19 is a table with information on the T cell donors used in the disclosed study.

FIG. 20 is a list of the dCODE Dextramer reagents used in the disclosed study and NetMHC peptide HLA allele binding prediction.

FIG. 21 is a table with a summary of pMHC-TCR binding events.

FIG. 22 shows TCR-pMHC repertoire diversities and peptide properties.

FIG. 23 shows a summary of 11 pMHC repertoires collated from VDJdb and McPAS.

FIG. 24 shows specificities of expanded TCR clonotypes pMHC in binders identified by 10× Genomics. The 50 largest TCR clones from donors 1 to 4 are plotted along with their binding specificities and concordance. A circle indicates that at least one member of the clonotype was classified as specific for a particular pMHC. Circle size indicates the total within-donor clonotype size. Circle color indicates the proportion of cells within the clonotype that bind the Dextramer (the ‘binding concordance’).

FIGS. 25A-G shows the identification and characterization of pMHC binding T cells from the high-throughput pMHC binding data. (A) The ICON (Integrative COntext-specific Normalization) workflow schema. RT: the fraction of T cells within a clone binding to a particular dextramer; RC: the ratio of a dextramer signal within a cell to the sum of all dextramers binding to the cell. (B) The pMHC binding landscape network of ICON identified dextramer binders. Each node represents a pMHC repertoire and is displayed as a pie chart of the number of unique paired TCRs from each donor that bind to that pMHC. The node size denotes the total number of unique TCRs that bind to a given pMHC. Each edge represents unique TCR(s) shared by two pMHCs. The thickness of an edge represents the number of shared unique TCR(s). (C) The correlation of the result from flow sorting on single dextramer binding and ICON estimated relative abundance of pMHC binding T cells. The number of the dextramers for the validation is 21. (D) The uniqueness and overlap of pMHC binding TCRs identified among donors 1, 2, 3, 4 and V. (E) The majority of identified binders interact with the nine pMHCs. (F) V and J gene segment usages for T cells binding to these nine pMHCs. The gene segments with less than 5% were combined and indicated in grey. (G) HLA type restricted and unrestricted bindings.

FIG. 26A-D shows processing the high-throughput data using ICON. (A) Scatter plots of the number of detected genes versus percentage of mitochondria gene expression using single cell RNA data. Each dot represents a cell. Dots in red are dead cells or doublets. (B) The distributions of dextramer signals in UMI from negative control and test dextramers. Sorted_nc: negative control dextramers; sorted_dex: test dextramers. (C) Scatter plot of RT versus RC. RC is the ratio of a dextramer signal within a cell to the sum of all dextramers binding to the T cell. RT is the fraction of T cells within a clone binding to a particular dextramer. (D) Hierarchy cluster of ICON identified pMHC binding T cells. Each row is a dextramer and column is a T cell.

FIG. 27 shows pooled dextramer FACS gating for fluorescence activated sorting (FACS) of dextramer⁺ T cells from donor V.

FIGS. 28A-B show single oligo-dextramer sorting. (A) Representative gating for fluorescence activated sorting (FACS) of dextramer positive T cells. T cells were previously enriched from Donor V peripheral blood mononuclear cells (PBMC) then stained with Single oligo-dextramer. The following sequential gating strategy was employed to isolate the desired dextramer+ population for sorting. (B) Scatter plots of single oligo-dextramer cell sorting results for each 21 test dextramers and two negative control dextramers.

FIG. 29 is a table showing a summary of pMHC-TCR binding events ICON identified from the high throughput pMHC binding data.

FIGS. 30A-B show a characterization of ICON identified pMHC binding T cells from the high throughput dataset. (A) Power law distributions of unique T cell clone sizes of the top nine most abundant pMHC binding T cell repertoires. (B) Shannon diversity scores of the top nine pMHC repertoires.

FIGS. 31A-C shows a TCRAI model and performance on the gold-standard dataset. (A) Schematic of the TCRAI framework for a model receiving input of CDR3, and V, J genes of both the α and β chains. A trained TCRAI model creates a numerical fingerprint, and prediction, for a given TCR. (B) ROC curves for TCRAI classification performance using the 8 curated public TCR-pMHC binding repertoires. Binders are unique TCRs that bind to a particular pMHC, and non-binders are unique TCRs that bind to other pMHCs. Paired α & β TCR sequences were used as input data. FPR: false positive rate; TPR: true positive rate. (C) Classification performance comparison. TCRAI was compared with predictive classifiers NetTCR, TCRdist and DeepTCR. The area under the ROC curve (AUC) scores for NetTCR and TCRdist were generated using the original classifiers with default parameters. The AUC score for DeepTCR (a multinomial classifier) was derived from a slightly modified and hyperparameters optimized version of DeepTCR (Methods) in order to compare with these binomial classifiers NetTCR and TCRdist. For the comparison, the binomial mode of TCRAI was used.

FIGS. 32A-C shows ROC performance of TCR antigen specificity classifiers (a and b). (c) shows ROC curves for TCRAI in multinomial mode using the nine pMHC binding repertoires identified from the high-throughput dataset. Paired α and β TCR sequences were used as input data. FPR: false positive rate; TPR: true positive rate.

FIG. 33 is a table showing the comparison of TCR-antigen specificity classifiers.

FIGS. 34A-D shows TCRAI performance on the high-throughput dataset. (A) ROC curves for TCRAI on the top nine most abundant pMHC binding repertoires. Binders are unique TCRs that bind to a particular pMHC, and non-binders are unique TCRs that bind to other pMHCs. Paired α & β TCR sequences were used as input data. FPR: false positive rate; TPR: true positive rate. (B) Classification performance comparisons using TCRα only, TCRβ only or paired TCRα & β chains as the sequence input. (C) ROC curves from the independent test of four overlapping pMHC repertoires between the curated public dataset and the high throughput dataset. TCRAI was trained by pMHC repertoires identified from the high throughput dataset and tested on the curated public dataset. (D) UMAP of both the training (high-throughput data) and the testing (“gold-standard” data) TCRAI fingerprints extracted from the high-throughput trained models. The left panel shows the strong overlap between A*02:01_ELAGIGILTV_MART-1_Cancer training and testing sets, while the poor overlap of A*02:01_NLVPMVATV_pp65_CMV training and testing datasets is shown in the right panel. The black circle highlights the region with almost no overlap fingerprints of binding TCRs.

FIG. 35 shows ROC curves for TCRAI in multinomial mode using the nine pMHC binding repertoires identified from the high-throughput dataset. Paired α & β TCR sequences were used as input data. FPR: false positive rate; TPR: true positive rate.

FIGS. 36A-B shows TCRAI fingerprint comparison between models trained on different datasets. (A) Comparison of high-throughput and “gold-standard” TCR fingerprints generated by a high-throughput data trained model for the two cases not shown in FIG. 3d, showing a good overlap in binders in both cases. (B) The inference problem was performed in reverse: training a model with the “gold-standard” data and calculating fingerprints of the “gold-standard” and high-throughput TCRs. For the case of A*02:01_NLVPMVATV_pp65/CMV, where cross-dataset performance was poor, the model trained on the “gold-standard” data containing TCRs from many donors separates a large group of binding TCRs. However, the high-throughput binding TCRs come from predominantly a single donor, who only has binding TCRs from small clusters in TCR space that do not well represent the range of binding TCRs occurring in the wider population. The black circle highlights the TCRs unique to the high-throughput data.

FIGS. 37A-G shows a characterization of TCR groups. (A) Clustering TCRAI fingerprints of high-confidence TCRs identified from the high throughput dataset by a model trained to predict A*02:01_GILGFVFTL_Flu-MP_Influenza binders reveals two TCR clusters: cluster 0 (orange) and cluster 1 (green). (B) The dextramer signal (in UMI) distributions of clusters 0 and 1. (C) Conserved CDR3 motifs and gene usage in these two clusters of Flu peptide binding TCRs. For cluster 0 the gene usage was shown for the 30 most common unique quadruplets of gene-usage such that the key variability can be seen in one plot. (D) 3D structures of Flu peptide binding TCR-pMHC binding complexes for a cluster 0 TCR (PDB 2VLJ) and a cluster 1 TCR (PDB 5JHD). In the top panels, only non-peptide residues within 4 Å of the Phe-5 ring (β-chain in pink, α-chain in blue, MHC in green) are shown. In the bottom panel, a comparison of peptide structures from cluster 0 and cluster 1 TCR-pMHC binding complexes. (E) Clustering of TCRAI fingerprints of TCRs with a high-confidence to bind A*02-01_GLCTLVAML_BMLF1_EBV from the high throughput dataset. (F) Dextramer signal (in UMI) distributions of EBV peptide binding clusters 0 to 2. (G) Conserved CDR3 motifs and gene usage in these three clusters of EBV peptide binding TCRs.

FIGS. 38A-F shows immune phenotypes of pMHC binding CD8+ T cells. (A) Classification of pMHC binding cells. Clusters were visualized by UMAP and cell types were represented by different colors. (B) The heatmap of the expression of CD8+ T cell type marker genes and proteins. *: protein expression measured by CITE-seq. (C) pMHC binding landscape by T cell immune subtypes. Bars indicate the number of pMHC binding T cells in log 2 scale. (D) Expanded clonotypes are enriched in the non-naïve compartment. Each dot represents a unique TCR clone. (E) The pie chart describes subpopulations of pMHC binding CD8+ T cells. (F) Proportion of HLA matched and mismatched binding in naïve and non-naïve binding T cells. Tpm: peripheral memory cells; Tcm: central memory cells; Tem: effector memory cells; Temra: terminally differentiated effector memory cells; Others: other memory cells with marker expression CD43^{lo}KLRG1^{hi}CD127.

FIG. 39 shows the importance of VJ gene information. Errors in AUC when comparing models trained using full input or only gene input are calculated by propagating the errors on the AUC for each model (full or gene), with the assumption of no covariance between the results. Error on the AUC for each model is either the difference between mean AUC for the best hyperparameters during MCCV and the final model trained with those hyperparameters, or the standard deviation of AUC during MCCV, whichever was larger. ΔAUC=AUC_{full}−AUC_{gene}.

FIGS. 40A-B shows a characterization of TCR groups. (A) The dextramer signal distributions of all 5 TCR clusters identified for A*02-01_GLCTLVAML_BMLF1_EBV as shown in fingerprint space in FIG. 4e. (B) The motif and gene usage of EBV peptide binding TCR clusters 3 and 4.

FIG. 41 shows an example operational environment.

FIG. 42 shows an example method.

FIG. 43 shows an example method.

FIG. 44 shows an example method.

FIG. 45 shows an example method.

FIG. 46 shows an example method.

### DETAILED DESCRIPTION

The disclosed method and compositions may be understood more readily by reference to the following detailed description of particular embodiments and the Example included therein and to the Figures and their previous and following description.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a.”, “an.” and “the” include plural reference unless the context clearly dictates otherwise. Thus, for example, reference to “a TCR” includes a plurality of such TCRs, reference to “the dextramer” is a reference to one or more dextramers and equivalents thereof known to those skilled in the art, and so forth.

The term “subject” or “donor” may refer to an animal, such as a mammalian species (preferably human) or avian (e.g., bird) species. More specifically, a subject or donor can be a vertebrate, e.g., a mammal such as a mouse, a primate, a simian or a human. Animals include farm animals, sport animals, and pets. A subject or donor can be a healthy individual, an individual that has symptoms or signs or is suspected of having a disease or a predisposition to the disease, or an individual that is in need of therapy or suspected of needing therapy. In some embodiments, the subject donor is human, such as a human who has, or is suspected of having, cancer.

The term “barcode,” as used herein, generally refers to a label that may be attached to a molecule (e.g., dextramer, cell) to convey information about the molecule. For example, a DNA barcode can be a polynucleotide sequence attached to each dextramer and a common sequencing barcode can be a polynucleotide sequence attached during sequencing. This barcode can then be sequenced. The presence of the same barcode on multiple sequences may provide information about the origin of the sequence. For example, a barcode may indicate that the sequence came from a particular dextramer. A barcode can also indicate that a sequence came from a particular cell/dextramer combination.

As used herein, the terms “sequencing” or “sequencer” refer to any of a number of technologies used to determine the sequence of a biomolecule, e.g., a nucleic acid such as DNA or RNA. Exemplary sequencing methods include, but are not limited to, targeted sequencing, single molecule real-time sequencing, exon sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, and a combination thereof. In some embodiments, sequencing can be performed by a gene analyzer such as, for example, gene analyzers commercially available from Illumina or Applied Biosystems.

A “polynucleotide”, “nucleic acid”, “nucleic acid molecule”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Oligonucleotides often range in size from a few monomeric units, e.g. 3-4, to hundreds of monomeric units. Whenever a polynucleotide is represented by a sequence of letters, such as “ATGCCTG,” it will be understood that the nucleotides are in 5′→3′ order from left to right and that “A” denotes adenosine, “C” denotes cytosine, “G” denotes guanosine, and “T” denotes thymidine, unless otherwise noted. The letters A, C, G, and T may be used to refer to the bases themselves, to nucleosides, or to nucleotides comprising the bases, as is standard in the art.

The term “DNA (deoxyribonucleic acid)” refers to a chain of nucleotides comprising deoxyribonucleosides that each comprise one of four nucleobases, namely, adenine (A), thymine (T), cytosine (C), and guanine (G). The term “RNA (ribonucleic acid)” refers to a chain of nucleotides comprising four types of ribonucleosides that each comprise one of four nucleobases, namely; A, uracil (U), G, and C. Certain pairs of nucleotides specifically bind to one another in a complementary fashion (called complementary base pairing). In DNA, adenine (A) pairs with thymine (T) and cytosine (C) pairs with guanine (G). In RNA, adenine (A) pairs with uracil (U) and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “nucleotide sequence”, “genomic sequence,” “genetic sequence,” or “fragment sequence,” or “nucleic acid sequencing read” denotes any information or data that is indicative of the order of the nucleotide bases (e.g., adenine, guanine, cytosine, and thymine or uracil) in a molecule (e.g., a whole genome, whole transcriptome, exome, oligonucleotide, polynucleotide, or fragment) of a nucleic acid such as DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, and electronic signature-based systems.

“Optional” or “optionally” means that the subsequently described event, circumstance, or material may or may not occur or be present, and that the description includes instances where the event, circumstance, or material occurs or is present and instances where it does not occur or is not present.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

“Exemplary” means “an example of” and is not intended to convey an indication of a preferred or ideal configuration. “Such as” is not used in a restrictive sense, but for explanatory purposes.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

### B. Methods of Identifying Reliable Receptor-pMHC Binding and Uses Thereof

In some aspects, the methods and systems described can identify reliable TCR-pMHC bindings by analyzing multi-omics high-throughput binding data. The methods and systems may be referred to herein as ICON (Integrative COntext-specific Normalization).

Disclosed are methods of receiving single cell sequence data, dextramer sequence data, and single cell receptor sequence data; filtering, from the dextramer sequence data, based on the single cell sequence data, data associated with low-quality cells; adjusting, based on a measure of background noise, the dextramer sequence data; filtering, from the dextramer sequence data, based on the single cell receptor data, data according to a presence or an absence of specific receptor sequences; and identifying data remaining in the normalized filtered dextramer sequence data as associated with reliable receptor-pMHC binding events.

The single cell sequence data and corresponding receptor sequence data can be from several cell types, including T cells (αβ or γδ) and B cells. Thus, as an example, disclosed are methods of receiving single cell sequence data, dextramer sequence data, and single cell TCR sequence data; filtering, from the dextramer sequence data, based on the single cell sequence data, data associated with low-quality cells; adjusting, based on a measure of background noise, the dextramer sequence data; filtering, from the dextramer sequence data, based on the single cell TCR-data, data according to a presence or an absence of an α-chain or a β-chain; and identifying data remaining in the normalized filtered dextramer sequence data as associated with reliable TCR-pMHC binding.

1. Data Acquisition

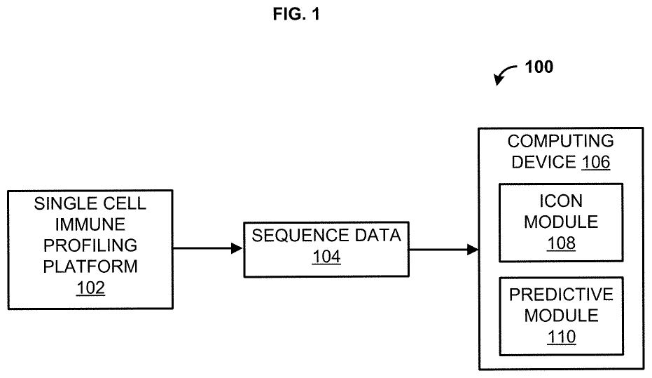

Disclosed are methods of acquiring, receiving, and/or determining multi-omics high-throughput binding data. As shown in FIG. 1, a system 100 can comprise a single-cell immune profiling platform 102. The single-cell immune profiling platform 102 may be configured to generate multi-omics high-throughput binding data (e.g., sequence data 104). In an aspect, the multi-omics high-throughput binding data can comprise one or more of single cell sequence data, dextramer sequence data, and/or single cell receptor sequence data. The single cell sequence data can comprise, for example, RNA-seq data. The dextramer sequence data can comprise, for example, dCODE-Dextramer-seq and/or cell surface protein expression sequencing, also referred to as CITE-seq (cellular indexing of transcriptomes and epitopes by sequencing). The single cell receptor sequence data can comprise, for example, TCR-seq data, such as paired αβ chain (or γδ chain) single cell TCR-seq data.

In some aspects, the multi-omics high-throughput binding data can be previously generated and incorporated into the disclosed methods. In some aspects, the multi-omics high-throughput binding data can be generated as part of the disclosed methods.

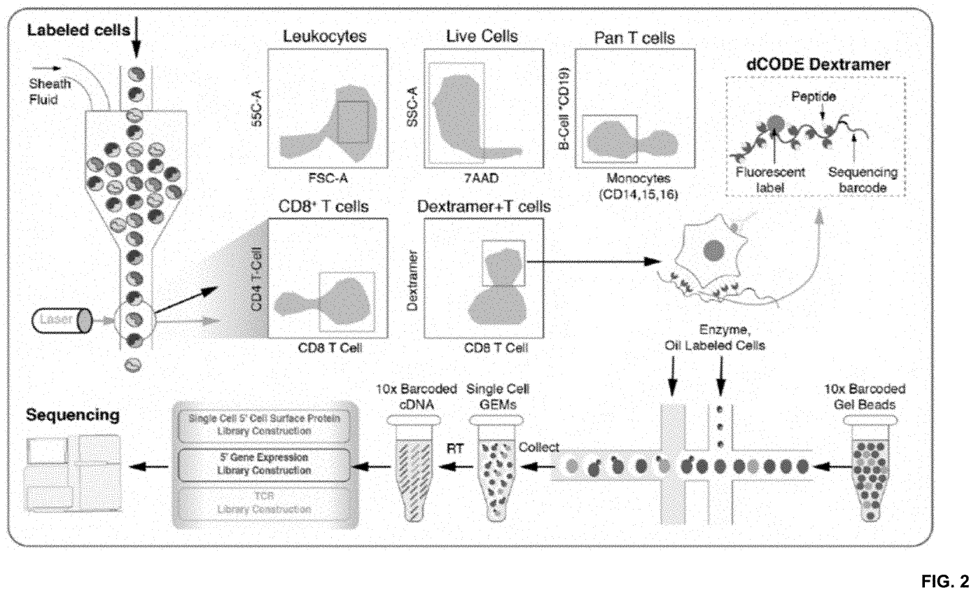

In some aspects, as shown in FIG. 2, the single-cell immune profiling platform 102 may be configured to label peripheral blood mononuclear cells (PBMCs) from healthy human donors for sorting on cells, such as, T cells or B cells. In some aspects, the cells can be T cells (e.g., CD4+ or CD8+ cells). In some aspects, the T cells can be αβ T cells or γδ T cells. In some aspects, the cells can be B cells. Thus, when labeling for sorting, the label can be a CD4, CD8, or B cell specific label.

In some aspects, once the cell type of interest has been sorted, the sorted cells can then be sorted for cells that bind a particular peptide-major histocompatibility complex (MHC) (pMHC). In some aspects, cells can be combined with a set of dextramers, for example, dCODE™ dextramers. In some aspects, the dCODE™ Dextramer® technology can be used. The dextramers can comprise two or more MHCs, a peptide presented by each MHC, and a DNA barcode. In some aspects, a pool of dextramers are used. In some aspect, a pool of dextramers can comprise, but is not limited to, 2, 3, 4, 5, 6, 7, 8, 9, 10, 15, 20, 25, 30, 35, 40, 45, 50, 55, 60, 65, 70, 75, 80, 85, 90, 95, or 100 single dextramers each comprising a different pMHC. In some aspects, a pool of dextramers comprises two or more of each of the single dextramers comprising a different pMHC. In some aspects, the two or more MHCs on a single dextramer are the same and therefore present the same peptide. In some aspects the MHC can be a MHC class I (MHC I) or MHC class II (MHC II). In some aspects, the DNA barcode comprises one or more primer sequences, a peptide-MHC (pMHC) specific barcode, and a unique molecular identifier. In some aspects, the dextramers can further comprise a label. For example, the label can be a fluorescent label. In some aspects, cells that bind a particular pMHC are sorted based on the label on the dextramer. In some aspects, cells that bind a particular pMHC are sorted based on a labeled antibody specific to the dextramer.

In some aspects, the cell sorting for specific cell types and the cell sorting for cells recognizing a dextramer can be performed simultaneously or consecutively.

In some aspects, after sorting of the cells that bound to dextramers comprising pMHCs, each cell and the corresponding dextramer can be sequenced. In some aspects, the cell sequence and the dextramer sequence (e.g., the DNA barcode sequence from the dextramer) all have a common sequencing barcode which allows one to determine which cell sequences were associated with which dextramer sequences. In some aspects, the Next GEM technology can be used for sequencing. The common sequencing barcode is different than the DNA barcode found on the dextramers.

In some aspects, the sequencing of the cells that bound to dextramers comprising pMHCs provides the sequence data 104 which may comprise single cell sequence data, dextramer sequence data, and single cell receptor sequence data. In some aspects, the single cell sequence data comprises sequences from the entire cellular genome or transcriptome. Thus, in some aspects, single cell sequence data comprises gene expression data. In some aspects, the dextramer sequence data comprises the DNA barcode sequence. In some aspects, single cell receptor sequence data comprises a sequence of a specific receptor. For example, single cell receptor sequence data comprises single cell TCR or B cell receptor (BCR) sequence data. In some aspects, single cell TCR sequence data comprises paired TCR sequence data. In some aspects, paired TCR sequence data comprises sequence data for the a chain and the β chain, if present, for each cell. In some aspects, paired TCR sequence data comprises sequence data for the γ chain and the δ chain, if present, for each cell. Thus, for each method and example described herein, the sequencing of the alpha chains and beta chains can be exchanged for sequencing of the gamma chains and delta chains.

Returning to the system 100 shown FIG. 1, in an aspect, the sequence data 104 may be provided to a computing device 106. The computing device 106 may be, for example, a smartphone, a tablet, a laptop computer, a desktop computer, a server computer, or the like. The computing device 106 may include a group of one or more servers. The computing device 106 may be configured to generate, store, maintain, and/or update various data structures including a database for storage of one or more of the sequence data 102. The computing device 106 may be configured to operate one or more application programs, such as an Integrative COntext-specific Normalization (ICON) module 108 and/or a predictive module 110. The ICON module 108 and the predictive module 110 may be stored and or configured to operate on the same computing device or separately on separate computing devices.

In some aspects, the ICON module 108 can be configured to analyze the received sequence data 104 (e.g., multi-omics high-throughput binding data, single cell sequence data, dextramer sequence data, single cell receptor sequence data, etc.). The sequence data 104 may include sequence information as well as meta information. The sequence data 104 can be stored in any suitable file format including, for example, VCF files, FASTA files or FASTQ files, as are known to those of skill in the art. FASTA and FASTQ are common file formats used to store raw sequence reads from high throughput sequencing. FASTQ files store an identifier for each sequence read, the sequence, and the quality score string of each read. FASTA files store the identifier and sequence only. Other file formats are contemplated.

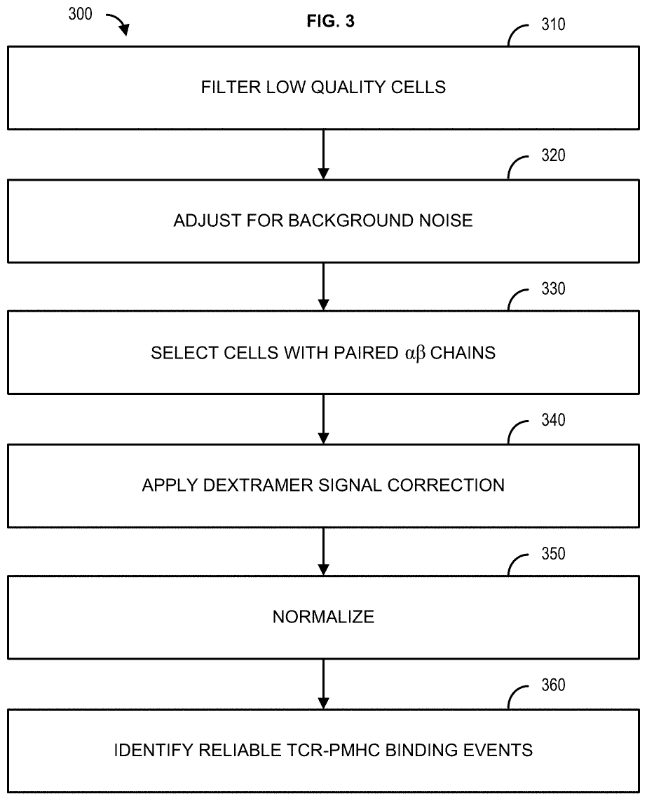

In some aspects, as shown in FIG. 3 the ICON module 108 can be configured to perform a method 300 comprising filtering of low-quality cells from the sequence data 104 (e.g., the dextramer sequence data) at step 310, adjusting the sequence data 104 for background noise at step 320, selecting T cells with paired αβ chains in the sequence data 104 at step 330, applying dextramer signal correction to the sequence data 104 at step 340, performing cell- and/or pMHC-wise dextramer signal normalization and binder identification to the sequence data 104 at step 350, and identifying data remaining in the normalized dextramer sequence data as associated with reliable TCR-pMHC binding events at step 360. In an embodiment, the ICON data process may be performed in a donor, cell, and/or dextramer specific context.

Filtering of low-quality cells from the sequence data 104 at step 310 may comprise single cell RNA-seq based filtering of low-quality cells. The ICON module 108 can be configured to filter out low quality cells such as doublets and dead cells. The cells with an unexpected high number of genes for T cells detected (e.g. >2500 genes per cell) may be categorized as doublets and cells with a high fraction of mitochondrial gene expression (e.g. ratio of mitochondrial gene expression UMIs to the total gene expression UMIs>0.4) or too few numbers of genes detected (<200 genes per cell) may be classified as dead cells. Data associated with the low quality cells may be removed from the sequence data 104 (e.g., the dextramer sequence data).

In an embodiment, filtering of low-quality cells from the sequence data 104 at step 310 may comprise determining, for each cell represented in the dextramer sequence data, based on the single cell sequence data, a number of genes, removing, from the dextramer sequence data, data associated with cells having a number of genes outside of a gene threshold range (the gene threshold range may be, for example, about 200 to about 2,500 genes), determining, for each cell represented in the dextramer sequence data, based on the single cell sequence data, a fraction of mitochondrial gene expression, and removing, from the dextramer sequence data, data associated with cells having a fraction of mitochondrial gene expression that exceeds a gene expression threshold. The gene expression threshold can be about 40 percent of total unique molecular identifier counts.

Adjusting the sequence data 104 for background noise at step 320 may comprise single cell dCODE-Dextramer-seq based background adjustment. In an aspect, two types of background noise controls that were designed for the dextramer binding assays include negative control dextramers from dextramer stained and sorted CD8+ T cells (NC_dex, denoted as nc), and the dextramer stained CD8+ T cells without sorting on dextramer (Dex_unsorted, denoted as du). To inspect signal and noise distributions, the maximum dextramer signal in UMI (Unique Molecular Identifier) of each cell may be selected to represent the best binding of each cell. Specifically, the non-specific dextramer binding signal of a cell may be represented as Max(nc₁, . . . , ncₙ), the maximum dextramer signal of n negative control dextramers included the dextramer pool. The dextramer binding signal of a cell from a dextramer stained and sorted sample (Dex_sorted, denoted as ds) may be represented as Max(ds₁, . . . , dsₘ), the maximum dextramer signal in UMI of m testing dextramers. Similarly, the dextramer binding signal of a cell from a Dex_unsorted sample may be represented as Max(du₁, . . . , duₘ). P_{99.9} of the non-specific dextramer binding signals in UMI may be selected as a non-specific dextramer binding cutoff (absolute outliers of negative dextramer controls may be excluded).

To estimate the potential noise introduced by the cell sorting process, the accumulative distributions of dextramer binding signals between Dex_sorted and Dex_unsorted samples may be compared to determine a cutoff for dextramer sorting efficiency. Kolmogorov-Smirnov test (KS test) p-values may be calculated by comparing the accumulative curves of dextramer sorted and dextramer unsorted samples using each data point (dextramer UMI) as a sliding window. The dextramer UMI which defines the largest difference of dextramer binding signals between Dex_sorted and Dex_unsorted (argmax D_{s,u}) may be used as a threshold for estimating dextramer sorting efficiency. The measure of estimated background noise (d) of dextramer sorted samples may be defined as:

d=Max(P_{99.9},argmax D_{s,u})

The dextramer signals (UMI) for each testing dextramer of sorted cells may be corrected by subtracting the measure of estimated background noise (d):

E_{c}=Eₛ−d

In an embodiment, adjusting the data for background noise at step 320 may comprise determining, based on the dextramer sequence data, sorted dextramer sequence data and unsorted dextramer sequence data. The sorted dextramer sequence data can comprise sorted test dextramer sequence data (dex_sorted) and negative control dextramer sequence data (nc_dex). The unsorted dextramer sequence data, can comprise unsorted test dextramer sequence data (dex_unsorted). The method 300, at step 320, may determine, for each cell represented in the dextramer sequence data, based on the negative control dextramer sequence data (nc_dex), a maximum negative control dextramer signal (Max(nc₁, . . . , ncₙ)). The method 300, at step 320, may determine, for each cell represented in the dextramer sequence data, based on the sorted test dextramer sequence data (dex_sorted), a maximum sorted dextramer signal (Max(ds₁, . . . , dsₘ)). The method 300, at step 320, may determine, for each cell represented in the dextramer sequence data, based on the unsorted test dextramer sequence data (dex_unsorted), a maximum unsorted dextramer signal (Max(du, . . . , duₘ)).

The method 300, at step 320, may estimate, based on the maximum negative control dextramer signals, a dextramer binding background noise (P_{99.9}) and estimate, based on the maximum sorted dextramer signals and the maximum unsorted dextramer signals, a dextramer sorting gate efficiency (argmax D_{s, u}). The dextramer sorting gate efficiency may be determined, for example, by the maximum difference between Max(ds₁, . . . , dsₘ) of the sorted test dextramer sequence data and Max(du, . . . , duₘ) of the unsorted dextramer sequence data.

The method 300, at step 320, may determine, based on the dextramer binding background noise (P_{99.9}) and the dextramer sorting gate efficiency (argmax D_{s, u}), a measure of background noise (d) and subtract, for each cell represented in the dextramer sequence data, the measure of background noise (d) from a dextramer signal associated with each cell (E_{c}=Eₛ−d).

In an embodiment, selecting T cells with paired αβ chains in the sequence data 104 at step 330 may comprise determining, for each cell represented in the dextramer sequence data, based on the single cell TCR sequence data, a presence or an absence of at least one α-chain and at least one β-chain and removing, from the dextramer sequence data, based on the presence or the absence of the at least one α-chain and the at least one β-chain, data associated with cells having only an α-chain, only a β-chain, or multiple α- or β-chains. Step 330 may comprise removing any data from the dextramer sequence data that is not associated with cells with single paired γδ chains. Thus, the same steps for adjusting background noise at step 320 can be performed with regards to the presence or absence of the γ chain and/or δ chain.

Selecting T cells with paired αβ chains in the sequence data 104 at step 330 may comprise removing any data from the dextramer sequence data that is not associated with cells with single paired αβ chains. The single cell receptor sequence data (e.g., single cell TCR-seq data), may be used to determine data associated with T cells that have only α-chain, only β-chain, and multiple α- or β-chains and such data may be removed from the sequence data 104 (e.g., the dextramer sequence data). For T cells with multiple α- or β-chains detected, the α- or β-chains with highest UMI counts may be assigned to each T cell. For example, if one T cell has 4 α-chains and 4 β-chains detected, from the list of all β-chains, the β-chain with the highest UMI may be selected. Similarly for α-chains. The selected α- or β-chains from this process may be assigned to the cell.

The method 300, at step 340, may comprise applying dextramer signal correction to the sequence data 104. At step 340, dextramer signals in the sequence data 104 may be corrected, resulting in corrected dextramer sequence data. Each dextramer has an optimal binding condition, however it is impossible to arrange the experimental conditions such that a multiplexed dextramer binding assay is optimal for every dextramer. This results in multiple dextramers binding to the same T cell/clone. To correct for this effect, dextramer signals may be penalized if simultaneously binding to the same T cell/clone, using the following technique.

Defining the background noise subtracted dextramer signal for the i^{th} T cell binding the j^{th} dextramer as Eᵢⱼ, further denote the fraction of dextramer signal due to binding of the j^{th} dextramer for the i^{th} T cell as:

$${RC}_{ij} = \frac{{E}_{ij}}{{\sum }_{{j}^{′} = 1}^{n} {e}_{{ij}^{′}}} .$$

Denoting the TCR clonotype of the i^{th} T cell as kᵢ, and the number of T cells belonging to clonotype kᵢ that bind dextramer j as Tₖ_{ᵢ}ⱼ, denote the fraction of T cells that belong to clonotype kᵢ that bind the j^{th} dextramer as:

$${RT}_{{k}_{i} j} = \frac{{T}_{{k}_{i} j}}{{\sum }_{{j}^{′} = 1}^{n} {T}_{{k}_{i} {j}^{′}}} .$$

Using these quantities, calculate the corrected dextramer signal for the i^{th} T cell binding the j^{th} dextramer as:

Sᵢⱼ=Eᵢⱼ(RCᵢⱼ)²RTₖⱼ.

The method 300, at step 350, may normalize the corrected dextramer sequence data by performing, for each cell represented in the dextramer sequence data, cell-wise normalization on the dextramer signals associated with each cell and/or performing, for each cell represented in the dextramer sequence data, pMHC-wise normalization. Such normalization can result in normalized dextramer sequence data. Step 350 may further comprise binder identification. To make all the dextramer binding signals comparable, the corrected dextramer binding signals may be log-ratio normalized across 44 testing dextramers within a cell. pMHC-wise normalization may subsequently be conducted based on Log-Rank distribution. Normalized dextramer UMI>0 was empirically chosen as the cutoff for pMHC specific binders.

In an embodiment, the corrected dextramer sequence data may be normalized at step 350. For example, a cell-wise normalization may be performed based on Log-Rank distribution for each cell and/or a pMHC-wise normalization may be performed to make the dextramer binding signals comparable to each other. The adjusted dextramer binding signals of sorted cells E_{c} may normalized across the testing dextramers, then across all cells as the following equation:

$${E}_{c}^{′} = \frac{log ( {E}_{c}^{ij} , 10 )}{{\sum }_{j = 1}^{j = N} log ( {E}_{c}^{ij} , 10 )}$$

$${E}^{*} = \frac{{{E}^{′}}_{c}^{ij} - {\overline{{E}^{′}}}_{c}^{j}}{σ ( {E}^{{′}_{c}^{{{ }^{′}}^{′} j}} )}$$

E_{c}′>=0.9 may be empirically determined as a cutoff for pMHC specific binders

The method 300, at step 360, may further identify data remaining in the normalized dextramer sequence data as associated with reliable TCR-pMHC binding events. Such data may be considered a portion of a training data set for use in a machine learning process. The resulting processed sequence data 104 (e.g., the training data set) may be provided to the predictive module 110.

### C. Methods of Using Reliable Receptor-pMHC Binding for Machine Learning

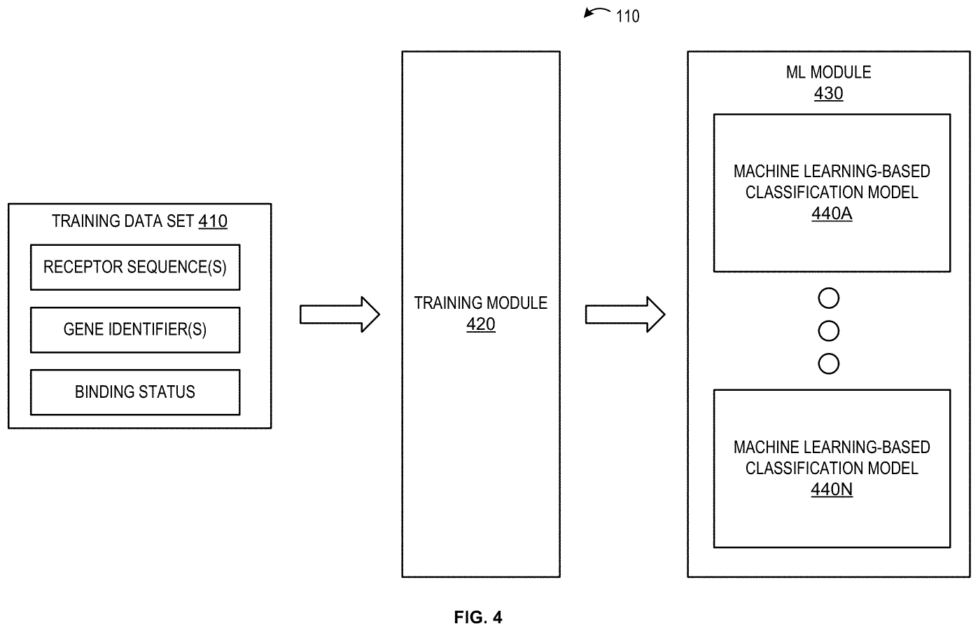

Turning now to FIG. 4, the predictive module 110 is described. The predictive module 110 may be configured to use machine learning (“ML”) techniques to train, based on an analysis of one or more training data sets 410 by a training module 420, at least one ML module 430 that is configured to predict a binding affinity for a given receptor sequence.

The training data set 410 may comprise one or more receptor sequences, one or more gene identifiers, a binding status, and an identifier of a peptide to which the receptor sequence bound (if any). The binding status may indicate “yes” for a receptor sequence that bound to a peptide or “no” for a receptor sequence that did not bind to a peptide. For receptor sequences that bound to a peptide, the identifier of the peptide can be used to identify an antigen associated with the peptide. Such data may be derived in whole or in part from the sequence data 104 processed by the ICON module 108. In an embodiment, TCR-CDR3 amino acid sequences may be determined from the sequence data 104, including associated V, D, and J gene identifiers, a label indicating binding status (Yes, No), and an identifier of a peptide to which the TCR-CDR3 amino acid sequences bound. The TCR-CDR3 amino acid sequences may be encoded into numbers to represent the 20 possible amino acids. Padding may be applied to sequences as needed. The V and J gene identifiers may be one-hot encoded to provide a categorical and discrete representation of the gene identifiers in numerical space. The encoded TCR-CDR3 amino acid and V and J gene identifiers may be concatenated together to represent one TCR record and associated with the label indicating binding status (Yes, No). The label may further indicate the specific peptide to which the TCR bound. One or more TCR records may be combined to result in the training data set 410.

A subset of the TCR records may be randomly assigned to the training data set 410 or to a testing data set. In some implementations, the assignment of data to a training data set or a testing data set may not be completely random. In this case, one or more criteria may be used during the assignment. In general, any suitable method may be used to assign the data to the training or testing data sets, while ensuring that the distributions of yes and no labels are somewhat similar in the training data set and the testing data set.

The training module 420 may train the ML module 430 by extracting a feature set from a plurality of TCR records (e.g., labeled as yes) in the training data set 410 according to one or more feature selection techniques. The training module 420 may train the ML module 430 by extracting a feature set from the training data set 410 that includes statistically significant features of positive examples (e.g., labeled as being yes) and statistically significant features of negative examples (e.g., labeled as being no).

The training module 420 may extract a feature set from the training data set 410 in a variety of ways. The training module 420 may perform feature extraction multiple times, each time using a different feature-extraction technique. In an example, the feature sets generated using the different techniques may each be used to generate different machine learning-based classification models 440. For example, the feature set with the highest quality metrics may be selected for use in training. The training module 420 may use the feature set(s) to build one or more machine learning-based classification models 440A-440N that are configured to indicate whether a new receptor sequence (e.g., with an unknown binding status) is likely or not likely to bind to a peptide or pMHC.

The training data set 410 may be analyzed to determine any dependencies, associations, and/or correlations between features and the yes/no labels in the training data set 410. The identified correlations may have the form of a list of features that are associated with different yes/no labels. The term “feature,” as used herein, may refer to any characteristic of an item of data that may be used to determine whether the item of data falls within one or more specific categories. By way of example, the features described herein may comprise one or more sequence patterns, amino acid sequences of one or both alpha and beta chains, names of v and j gene segments of one or both alpha and beta chains.

A feature selection technique may comprise one or more feature selection rules. The one or more feature selection rules may comprise an feature occurrence rule. The feature occurrence rule may comprise determining which features in the training data set 410 occur over a threshold number of times and identifying those features that satisfy the threshold as candidate features.

A single feature selection rule may be applied to select features or multiple feature selection rules may be applied to select features. The feature selection rules may be applied in a cascading fashion, with the feature selection rules being applied in a specific order and applied to the results of the previous rule. For example, the feature occurrence rule may be applied to the training data set 410 to generate a first list of features. A final list of candidate features may be analyzed according to additional feature selection techniques to determine one or more candidate feature groups (e.g., groups of features that may be used to predict binding). Any suitable computational technique may be used to identify the candidate feature groups using any feature selection technique such as filter, wrapper, and/or embedded methods. One or more candidate feature groups may be selected according to a filter method. Filter methods include, for example, Pearson's correlation, linear discriminant analysis, analysis of variance (ANOVA), chi-square, combinations thereof, and the like. The selection of features according to filter methods are independent of any machine learning algorithms. Instead, features may be selected on the basis of scores in various statistical tests for their correlation with the outcome variable (e.g., yes/no).

As another example, one or more candidate feature groups may be selected according to a wrapper method. A wrapper method may be configured to use a subset of features and train a machine learning model using the subset of features. Based on the inferences that drawn from a previous model, features may be added and/or deleted from the subset. Wrapper methods include, for example, forward feature selection, backward feature elimination, recursive feature elimination, combinations thereof, and the like. As an example, forward feature selection may be used to identify one or more candidate feature groups. Forward feature selection is an iterative method that begins with no feature in the machine learning model. In each iteration, the feature which best improves the model is added until an addition of a new variable does not improve the performance of the machine learning model. As an example, backward elimination may be used to identify one or more candidate feature groups. Backward elimination is an iterative method that begins with all features in the machine learning model. In each iteration, the least significant feature is removed until no improvement is observed on removal of features. Recursive feature elimination may be used to identify one or more candidate feature groups. Recursive feature elimination is a greedy optimization algorithm which aims to find the best performing feature subset. Recursive feature elimination repeatedly creates models and keeps aside the best or the worst performing feature at each iteration. Recursive feature elimination constructs the next model with the features remaining until all the features are exhausted. Recursive feature elimination then ranks the features based on the order of their elimination.

As a further example, one or more candidate feature groups may be selected according to an embedded method. Embedded methods combine the qualities of filter and wrapper methods. Embedded methods include, for example, Least Absolute Shrinkage and Selection Operator (LASSO) and ridge regression which implement penalization functions to reduce overfitting. For example, LASSO regression performs L1 regularization which adds a penalty equivalent to absolute value of the magnitude of coefficients and ridge regression performs L2 regularization which adds a penalty equivalent to square of the magnitude of coefficients.

After the training module 420 has generated a feature set(s), the training module 420 may generate a machine learning-based classification model 440 based on the feature set(s). A machine learning-based classification model may refer to a complex mathematical model for data classification that is generated using machine-learning techniques. In one example, the machine learning-based classification model 440 may include a map of support vectors that represent boundary features. By way of example, boundary features may be selected from, and/or represent the highest-ranked features in, a feature set.

The training module 420 may use the feature sets extracted from the training data set 410 to build a machine learning-based classification model 440A-440N for each classification category (e.g., yes, no). In some examples, the machine learning-based classification models 440A-440N may be combined into a single machine learning-based classification model 440. Similarly, the ML module 430 may represent a single classifier containing a single or a plurality of machine learning-based classification models 440 and/or multiple classifiers containing a single or a plurality of machine learning-based classification models 440.

The extracted features (e.g., one or more candidate features) may be combined in a classification model trained using a machine learning approach such as discriminant analysis; decision tree; a nearest neighbor (NN) algorithm (e.g., k-NN models, replicator NN models, etc.); statistical algorithm (e.g., Bayesian networks, etc.); clustering algorithm (e.g., k-means, mean-shift, etc.); neural networks (e.g., reservoir networks, artificial neural networks, etc.); support vector machines (SVMs); logistic regression algorithms; linear regression algorithms; Markov models or chains; principal component analysis (PCA) (e.g., for linear models); multi-layer perceptron (MLP) ANNs (e.g., for non-linear models); replicating reservoir networks (e.g., for non-linear models, typically for time series); random forest classification; a combination thereof and/or the like. The resulting ML module 430 may comprise a decision rule or a mapping for each candidate feature to assign an binding status to a new receptor sequence.

In an embodiment, the training module 420 may train the machine learning-based classification models 440 as a convolutional neural network (CNN). The CNN may comprise at least one convolutional feature layer and three fully connected layers leading to a final classification layer (softmax). The final classification layer may finally be applied to combine the outputs of the fully connected layers using softmax functions as is known in the art.

The candidate feature(s) and the ML module 430 may be used to predict the binding statuses (and associated peptides) of a plurality of TCR records in the testing data set. In one example, the result for each TCR record includes a confidence level that corresponds to a likelihood or a probability that the receptor sequence will bind to a peptide. The confidence level may be a value between zero and one, and it may represent a likelihood that the receptor sequence belongs to a yes/no binding status with regard to one or more peptides. In one example, when there are two statuses (e.g., yes and no), the confidence level may correspond to a value p, which refers to a likelihood that a particular receptor sequence belongs to the first status (e.g., yes). In this case, the a value 1-p may refer to a likelihood that the particular receptor sequence belongs to the second status (e.g., no). In general, multiple confidence levels may be provided for each test receptor sequence and for each candidate feature when there are more than two statuses. A top performing candidate feature may be determined by comparing the result obtained for each test receptor sequence with the known yes/no binding status for each test receptor sequence. In general, the top performing candidate feature will have results that closely match the known yes/no binding statuses.

The top performing candidate feature(s) may be used to predict the yes/no binding status of a receptor sequence with regard to one or more peptides. For example, a new TCR sequence may be determined/received. The new TCR sequence may be provided to the ML module 430 which may, based on the top performing candidate feature, classify the new TCR sequence as either binding (yes) or not binding (no) and an indication of the binding peptide(s).

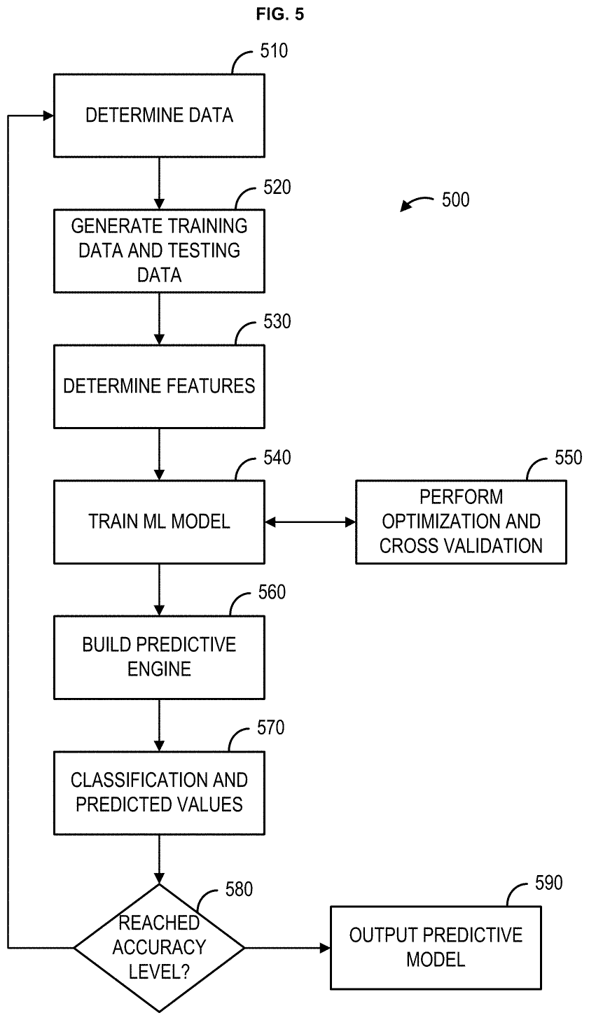

FIG. 5 is a flowchart illustrating an example training method 500 for generating the ML module 530 using the training module 420. The training module 420 can implement supervised, unsupervised, and/or semi-supervised (e.g., reinforcement based) machine learning-based classification models 440. The method 500 illustrated in FIG. 5 is an example of a supervised learning method; variations of this example of training method are discussed below, however, other training methods can be analogously implemented to train unsupervised and/or semi-supervised machine learning models.

The training method 500 may determine (e.g., access, receive, retrieve, etc.) first sequence data that has been processed by the ICON module 108 at step 510. The sequence data may comprise a labeled set of receptor sequences. The labels may correspond to binding status (e.g., yes or no) and identification of peptide(s) to which the receptor sequence bound.

The training method 500 may generate, at step 520, a training data set and a testing data set. The training data set and the testing data set may be generated by randomly assigning labeled receptor sequences to either the training data set or the testing data set. In some implementations, the assignment of labeled receptor sequences as training or testing samples may not be completely random. As an example, a majority of the labeled receptor sequences may be used to generate the training data set. For example, 75% of the labeled receptor sequences may be used to generate the training data set and 25% may be used to generate the testing data set.

The training method 500 may determine (e.g., extract, select, etc.), at step 530, one or more features that can be used by, for example, a classifier to differentiate among different classification of binding status (e.g., yes vs. no) with regard to one or more peptides. As an example, the training method 500 may determine a set features from the labeled receptor sequences. In a further example, a set of features may be determined from labeled receptor sequences different than the labeled receptor sequences in either the training data set or the testing data set. In other words, labeled receptor sequences may be used for feature determination, rather than for training a machine learning model. Such labeled receptor sequences may be used to determine an initial set of features, which may be further reduced using the training data set.

The training method 500 may train one or more machine learning models using the one or more features at step 540. In one example, the machine learning models may be trained using supervised learning. In another example, other machine learning techniques may be employed, including unsupervised learning and semi-supervised. The machine learning models trained at 540 may be selected based on different criteria depending on the problem to be solved and/or data available in the training data set. For example, machine learning classifiers can suffer from different degrees of bias. Accordingly, more than one machine learning model can be trained at 540, optimized, improved, and cross-validated at step 550.

The training method 500 may select one or more machine learning models to build a predictive model at 560. The predictive model may be evaluated using the testing data set. The predictive model may analyze the testing data set and generate predicted binding statuses at step 570. Predicted binding statuses may be evaluated at step 580 to determine whether such values have achieved a desired accuracy level. Performance of the predictive model may be evaluated in a number of ways based on a number of true positives, false positives, true negatives, and/or false negatives classifications of the plurality of data points indicated by the predictive model.

For example, the false positives of the predictive model may refer to a number of times the predictive model incorrectly classified a receptor sequence as binding that was in reality not binding. Conversely, the false negatives of the predictive model may refer to a number of times the machine learning model classified an receptor sequence as not binding when, in fact, the receptor sequence was binding. True negatives and true positives may refer to a number of times the predictive model correctly classified one or more receptor sequences as binding or non-binding. Related to these measurements are the concepts of recall and precision. Generally, recall refers to a ratio of true positives to a sum of true positives and false negatives, which quantifies a sensitivity of the predictive model. Similarly, precision refers to a ratio of true positives a sum of true and false positives. When such a desired accuracy level is reached, the training phase ends and the predictive model (e.g., the ML module 430) may be output at step 590; when the desired accuracy level is not reached, however, then a subsequent iteration of the training method 500 may be performed starting at step 510 with variations such as, for example, considering a larger collection of sequence data.

In an embodiment, provided is a flexible framework for the study of TCR-pMHC specificity, referred to herein as TCRAI. In an embodiment, TCRAI may utilize Tensorflow 2. TCRAI is highly modularized and allows for adjustment to model architecture. Any number of V(D)J genes and CDR regions of the TCR may be defined as inputs to the model in textual form. A selection may be made with regard to how to process these inputs into numerical form in a non-learnable way, via “processor” objects that convert text to numerical representations. These numerical inputs can then be further processed in learnable ways via “extractor” objects that form blocks of the neural network and give as their output vector representations of the input data, referred to herein as TCRAI fingerprints. TCRAI fingerprints may be concatenated into a single TCRAI fingerprint describing the input TCR via a single numerical vector. The TCRAI fingerprint may then be passed through a “closer” object which forms the final block of the neural network architecture, producing a prediction on the input TCR. TCRAI provides several such pre-built processors, extractors, and closers. TCRAI may be configured to perform binomial, multinomial, regression, and/or other tasks by choosing to construct a different closer object. In an embodiment, TCRAI may be used to build a model to make predictions of whether a given TCR can bind a specific pMHC complex.

In an embodiment, TCRAI may make use of 1D convolutions and batch normalization for CDR3 sequences and lower dimensional representations for genes, resulting in model regularization and forcing the model to learn stronger gene associations.

In an embodiment, the input information of the TCR may be processed into a numerical format. For each CDR3 sequence, amino acids may be converted to integers, and the integer vectors may be encoded into a one-hot representation. For V and J genes, a dictionary of gene type to integer may be built for each V and J gene and used to convert each gene to an integer.

The neural network architecture applied to the processed input information may include embedding layers and convolutional networks. Specifically, processed CDR3 residues may be embedded into a 16-dimensional space via a learned embedding, and the resulting numeric CDR3s may be fed through one or more (e.g., 3) 1D convolutional layers. In an embodiment, filters of dimensions [64,128,256], kernel widths [5,4,4], and strides [1,3,3] may be used. Each convolution may be activated by an exponential linear unit activation and followed by dropout and batch normalization. Following these three convolutional blocks, global max pooling may be applied to the final features, this process encodes each CDR3 by a vector of length 256, a “CDR3 fingerprint.” The processed gene input for each gene may be one-hot encoded and embedded into a reduced dimensional space (e.g., 16 for V genes, and 8 for J genes) via a learned embedding, giving a “gene fingerprint” of each gene as a vector. The fingerprints of all selected CDR3s and genes may then be concatenated together into a single vector, a “TCRAI fingerprint.” The TCRAI fingerprint may be passed through one final full-connected layer to give binomial predictions (single output value, sigmoid activation), regression predictions (single output, no activation), or multinomial predictions (multiple output values, softmax activation).

In an embodiment, TCR sequencing files may be collected as a raw csv formatted multi-omics high-throughput binding data. Sequencing files may be parsed to take the amino acid sequence of the CDR3 after removing unproductive sequences. Clones with different nucleotide sequences, but the same matched amino acid sequence from CDR3s, and the V, D, J genes may be aggregated together under one TCR. Thus, each TCR record may include single paired α and β TCR chains, with CDR3 amino acid sequence and V, J genes for each chain.

The data may be split into a training set (e.g., 76.5%), a validation set (e.g., 13.5%), and a left-out test set (e.g., 10%) for each model, and subsequently a 5-fold Monte-Carlo cross-validation (MCCV) may be performed on the training set. The model may be trained by minimizing the cross-entropy loss via the Adam optimizer, and the cross-entropy loss may be weighted by weights 1/(number of classes*fraction of samples in that class) for each class. Early stopping may be engaged, via a left-out validation dataset, to prevent overfitting, in which the model ceases training if the validation loss increases for more than 5 epochs and the weights of the model with minimal validation loss are restored. In the event of training a large number of models, only the learning rate and batch size need be tuned during cross-validation. After cross-validation the optimally performing hyperparameters may be selected and the model may be re-trained on the full training set, using the validation set to control early-stopping. The re-trained model may then be evaluated on the left-out test set.

TCRAI models may produce both a prediction for a TCR to bind a specific pMHC (or one of many pMHCs, in the multinomial case), and a numerical vector (TCRAI fingerprint) (e.g., by encoding paired αβ chain CDR3 amino acid sequences and the V and J genes of each TCR into a one-dimensional input vector) that describes that TCR within the context of the question of whether it can bind that pMHC.

In an embodiment, the distribution of fingerprints may be analyzed to identify groups of TCRs with different binding modalities. The fingerprints can be reduced to a two-dimensional space, for example, using UMAP: Uniform Manifold Approximation and Projection for Dimension Reduction. When using a model trained on one dataset and inferring fingerprints on another unseen dataset, the UMAP projector can be fit with TCRs from the training dataset and transform the TCRs from the unseen set using that projector.

When clustering TCR fingerprints, the fingerprints of all TCRs of the dataset can be projected into two-dimensional space as described above, and then those TCRs that are strong true positives (STPs, binomial prediction >0.95) can be selected. These STPs can then be clustered, for example using a k-means classifier, in the two-dimensional space. Other clustering algorithms may be used. TCRs from within in each cluster can then collected and used to construct CDR3 motif logos (using weblogo), gene-usage, and/or UMI distributions by pairing the unique TCR clonotypes within the cluster with all repeated clonotypes in the high throughput data.

### D. Methods of Use

In an aspect, the trained predictive model (e.g., machine learning classifier) may be used to predict a binding status of a TCR sequence with regard to one or more peptides. A TCR sequence may be presented to the machine learning classifier. The machine learning classifier may predict a likelihood that the TCR sequence will bind to one or more specific peptides. Similarly, a plurality of TCR sequences may be presented to the machine learning classifier. The machine learning classifier may predict, for each TCR sequence in the plurality of TCR sequences, a likelihood that each TCR sequence will bind to one or more specific peptides. In an aspect, the machine learning classifier can generate a TCR-peptide map as shown in the example output below.

| TCR Sequence | Peptide | Binding Likelihood |
|---|---|---|
| TCR Sequence 1 | Peptide 1 | 99% |
| TCR Sequence 2 | Peptide 6 | 99% |
| TCR Sequence 2 | Peptide 18 | 97.5% |
| TCR Sequence 2 | Peptide 10 | 68% |
| TCR Sequence 3 | Peptide 4 | 88% |
| TCR Sequence 4 | Peptide 24 | 59% |

A TCR-peptide map thus generated may be used to rapidly identify peptides that a subject's TCR sequences are likely to bind to. A biological sample (e.g., blood) may be obtained from a subject, cells isolated, and sequenced. The subject's TCR sequences may be identified and compared to the TCR-peptide map to identify peptides most likely to bind to the subject's TCR sequences.

In some aspects, identifying and evaluating antigen-specific T cells can be used to better understand the activities of drugs in mono- and combination therapy settings, identify features of potent anti-tumor T cells, screen for immunogenic epitopes in a haplotype-relevant manner, develop new vaccine and TCR therapies, and develop peptide binding algorithms based on TCR sequence features.

In some aspects, disclosed are methods of identifying a subject using binding patterns of the subject's TCRs. For example, blood can be drawn (first blood draw), cells from the blood can be processed via a single cell-based immune profiling platform, and the resulting data can be processed according to the ICON methods described herein. In some aspects, the cells are exposed to a variety of dextramers comprising pMHCs from a wide range of immunogens. After performing an ICON method as described herein, a reliable TCR binding pattern can be determined. In some aspects, a TCR binding pattern represents the specificity of TCRs to the immunogens on the dextramers. Blood can then be drawn at a different time point (days, weeks, months, years later) from the first blood draw (second blood draw). In some aspects, it would be expected that the second blood draw would likely comprise T cells having TCRs with different sequences than what was present in the first blood draw since there are about 10¹⁵ possible TCR sequences, however, the TCR binding pattern is unlikely to change. The cells from the second blood draw can be exposed to the same dextramers as used for the first blood draw and the resulting data analyzed according to an ICON method. Regardless of the different TCR sequences, the binding data of the first blood draw and second blood draw can be compared and used to determine if they are both from the same subject.

In some aspects, disclosed are methods of identifying a subject using machine learning to predict the binding patterns of the subject's TCRs. Reliable TCR binding data can be identified according to an ICON method as described herein. In some aspects, the reliable TCR binding data can be used to train a machine learning classifier as described herein. The trained machine learning classifier can be used to predict specificity TCR binding pattern of a subject. In some aspects, blood can be drawn (first blood draw) and a TCR binding pattern can be predicted using the trained machine learning classifier. Blood can then be drawn at a different time point (days, weeks, months, years later) from the first blood draw (second blood draw). In some aspects, it would be expected that the second blood draw would likely comprise T cells having TCRs with different sequences than what was present in the first blood draw since there are about 10¹⁵ possible TCR sequences, however, the TCR binding pattern is unlikely to change. Regardless of the different TCR sequences, the trained machine learning classifier may be used to predict a second TCR binding pattern using data derived from the second blood draw. It is possible to predict that the second blood draw is from the same subject as the first blood draw based on the TCR signatures.

In some aspects, a TCR or BCR binding pattern can be established using the described methods. In some aspects, having reliable TCR data identified using the methods described herein allows someone, such as a medical professional, to infer the antigenic history or vaccine history of a subject. In some aspects, reliable TCR data identified using the ICON methods described herein allows someone, such as a medical professional, to infer what pathogens a subject has been exposed to or even what countries the subject has visited. For example, the presence of TCR binding data to pathogens only present in Africa can indicate that the subject has been to Africa and been exposed to those pathogens.

In some aspects, reliable TCR data identified using the ICON methods described herein can assess a current immunologic state of a subject. For example, blood can be drawn (first blood draw), cells from the blood can be processed via a single cell-based immune profiling platform, and the resulting data can be processed according to the ICON methods described herein, resulting in TCR binding data. In some aspects, the dextramers used in establishing the TCR binding data comprise tumor specific pMHCs. Thus, once the TCR binding data has been normalized using an ICON method, and reliable TCR binding data is established, the presence of predicted tumor specific TCRs can be determined. For example, the reliable TCR data can be used in the disclosed machine learning (CNN) methods and therefore the blood from the subject can be analyzed for the presence of predicted tumor specific TCRs. Thus, the presence of tumor specific TCRs can result in early detection of cancer before any tumors or cancer symptoms are detected.

In some aspects, disclosed are methods for selecting T cells for T cell-based therapies. In some aspects, training data can be accumulated using the disclosed methods of machine learning classifying. In some aspects, the classifier can assign probabilities of a pMHC binding to each TCR sequence tested. In some aspects, the TCR sequence tested is associated with a T cell, wherein the T cell can be from a primary or secondary cell culture. This avoids needed to perform binding assays on all T cells being tested to determine if each T cell has a TCR specific to the different pMHCs. Instead, the classifier is relied on for determining the probability of TCR-pMHC binding. Those TCRs, and thus T cell comprising that TCR), classified as being highly selective to a specific pMHC can then be used for T cell therapies. In some aspects, T cells identified through the machine learning classifier can provide safer cell therapies than those T cells identified through binding assays because only the most reliable binding data was used to create the training data used to classify the TCRs associated with the T cells selected.

In some aspects, disclosed are methods for immune monitoring. In some aspects, blood can be drawn from a subject undergoing immunotherapy (e.g. vaccine treatment; immune checkpoint treatment), the cells, particularly the T cells, can be classified, based on the training data established in the disclosed machine learning approaches, as having a specificity to the epitope of interest or not. In some aspects, if a T cell is determined to have specificity to an epitope of interest then one can infer that the subject will be or is responsive to the immunotherapy. For example, if the immunotherapy is a vaccine that triggers an immune response to a cancer specific antigen, then T cells obtained from the subject would be classified based on their probability of binding to the cancer specific antigen. If T cells are selected as having a high probability of binding to the cancer specific antigen based on the training data obtained using the single cell immune profiling technology and ICON, then the subject would be considered to be a responder to the immunotherapy (e.g. vaccine).

In some aspects, disclosed are methods of TCR epitope mapping using the disclosed methods. In some aspects, TCR epitope mapping is a term that refers to the process of identifying the specific (in some cases the shortest) amino acid sequence of the epitope of a specific antigen that is recognized by T-cell (CD4+ and/or CD8+) receptors, and at the same time has the potential to stimulate a long lasting and a cytotoxic immune response. While performing the disclosed single cell immune profiling platform technology, dextramers can be used wherein all the different epitopes from one or more antigens of interest can be presented on dextramers. In other words, a single dextramer can comprise a pMHC wherein the peptide of the pMHC is a single epitope from one or more antigens of interest and enough dextramers are used so that every epitope of the one or more antigens of interest are present in the pMHC on the dextramers. T cells can be exposed to the dextramers in the disclosed single cell immune profiling platform with the dextramers comprising a single epitope from one or more antigens of interest and wherein enough dextramers are used so that every epitope of the one or more antigens of interest are present in the pMHC on the dextramers. The single cell sequence data, dextramer sequence data, and single cell TCR sequence data obtained from the single cell immune profiling can provide data about the T cells that bound to the different dextramers (e.g. epitopes). The single cell immune profiling data is then processed using ICON as described herein, therefore resulting in binding data for those cells that had the most reliable binding to one or more epitopes of the one or more antigens of interest. In some aspects, machine learning classification of TCRs that bind to the one or more epitopes of the one or more antigens of interest can be used to predict which T cells from a subject might be reactive against a particular antigen (e.g. tumor antigen).

### E. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for generating single cell sequencing data, the kit comprising reagents for single cell immune profiling. In some aspects, the kits can comprise one or more of the disclosed dextramers comprising pMHCs. In some aspects, the kits can comprise Next GEM sequencing materials. In some aspects, the kits can comprise multi-omics high-throughput binding data comprising one or more of single cell sequence data, dextramer sequence data, and/or single cell receptor sequence data.

### EXAMPLES

The following examples illustrate the present methods and systems as they relate to colorectal cancer detection. The following Examples are not intended to be limiting thereof.

### A. Example 1

1. Results

i. Multi-Omics High-Throughput TCR-pMHC Binding Data.

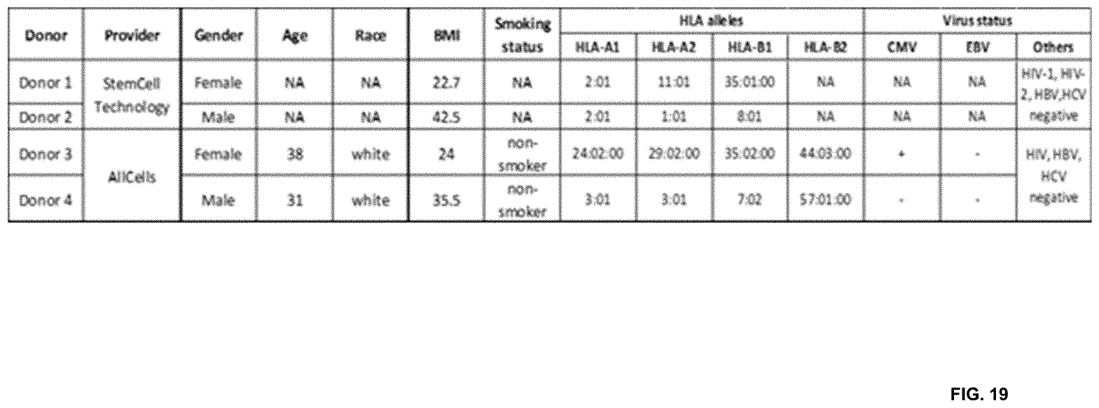

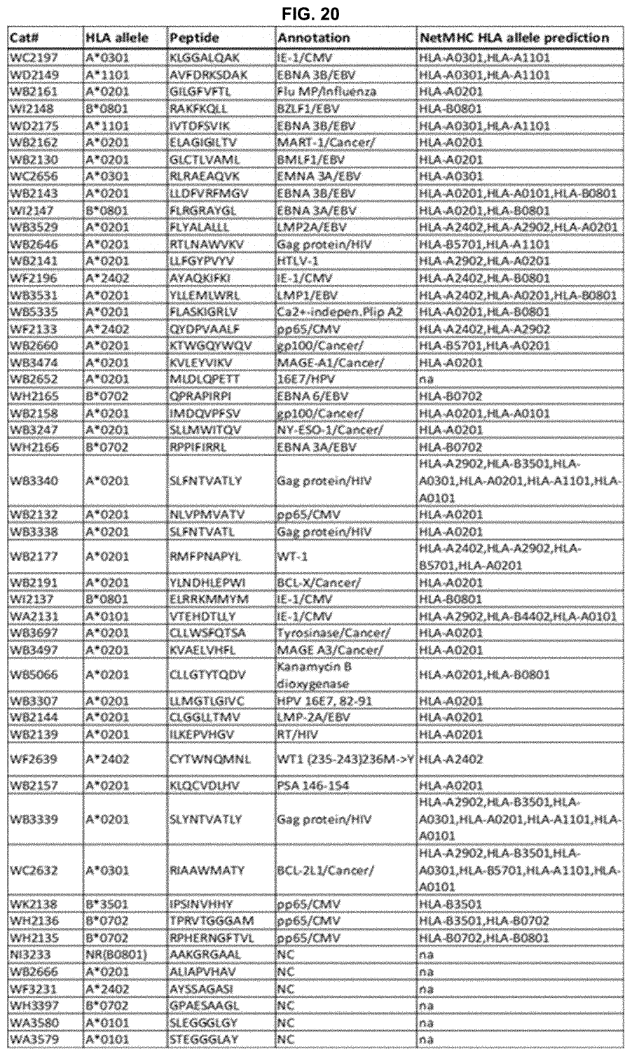

10× Genomics recently generated an expansive, publicly available TCR-pMHC binding dataset. In their initial report, the binding profile of over 150,000 CD8+ T cells from four HLA haplotyped healthy donors (FIG. 19) was assessed across 44 pMHC dextramers using a single cell-based immune profiling platform to directly detect antigen binding to T cells, while simultaneously sequencing T cell αβ chain pairs and transcriptomes (FIG. 2). The dextramer pool consists of epitopes with known common viral and cancer reactivities across eight HLA alleles (FIG. 20).

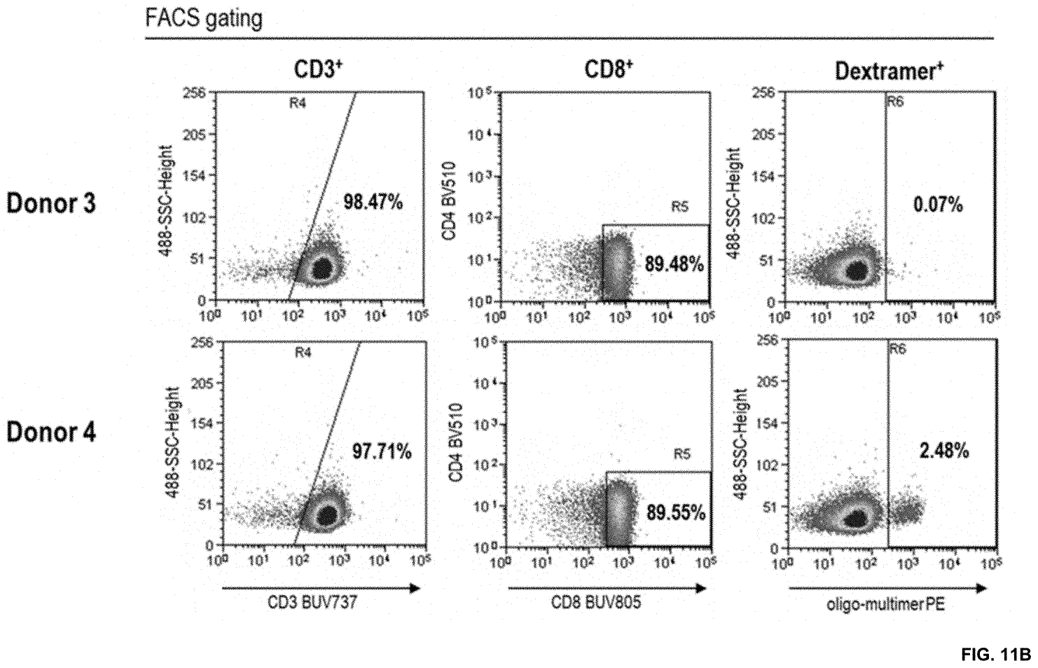

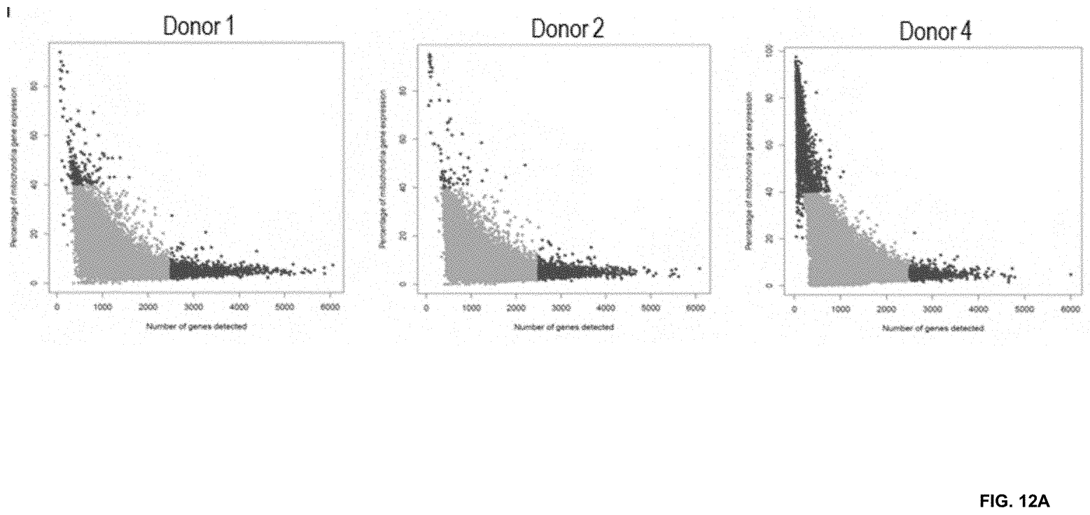

Described herein is a highly multiplexed dextramer binding dataset generated at the single cell level. 10× Genomics used a simple approach to determine pMHC binding TCRs by applying global cutoffs for background noise and non-specific dextramer binding to all donors. However, an unexpectedly high number of promiscuous cross-HLA and cross-peptide associations were found from the TCR-pMHC binding events identified by this approach, particularly in donors 3 and 4 (FIG. 11A). Upon further examination, the data from donor 3 were excluded from this study due to data quality issues (FIG. 11B).

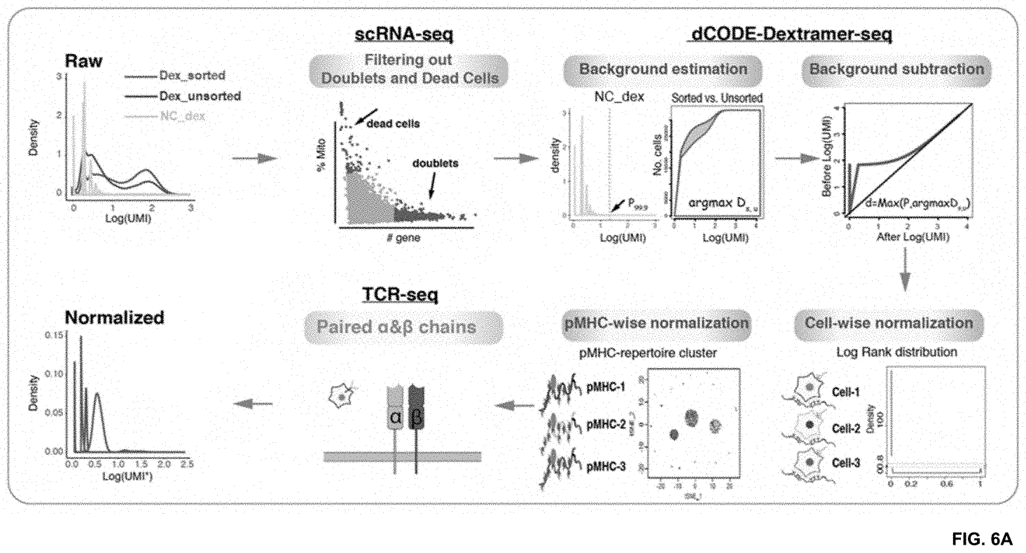

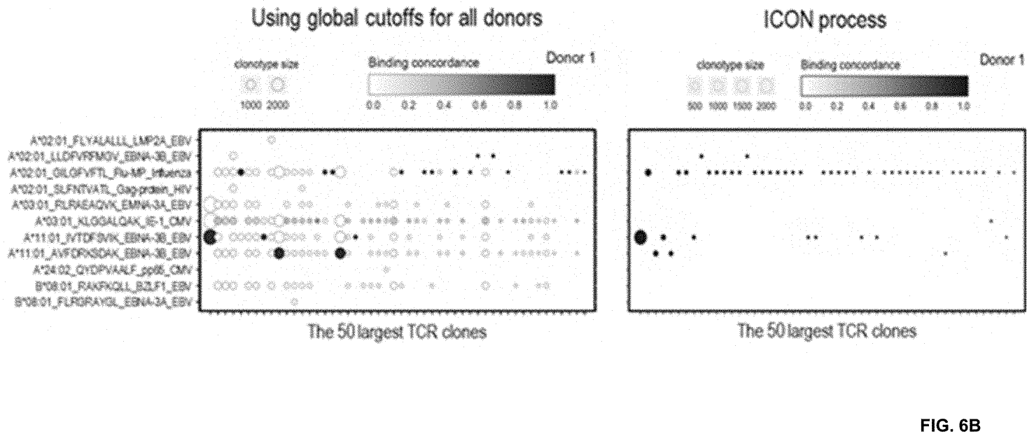

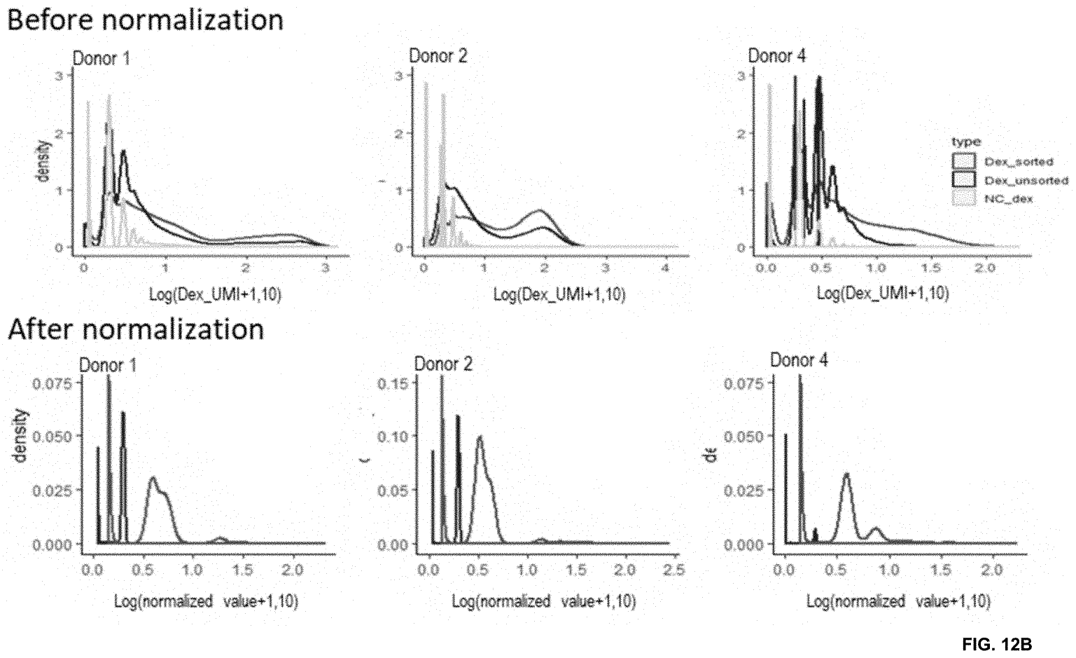

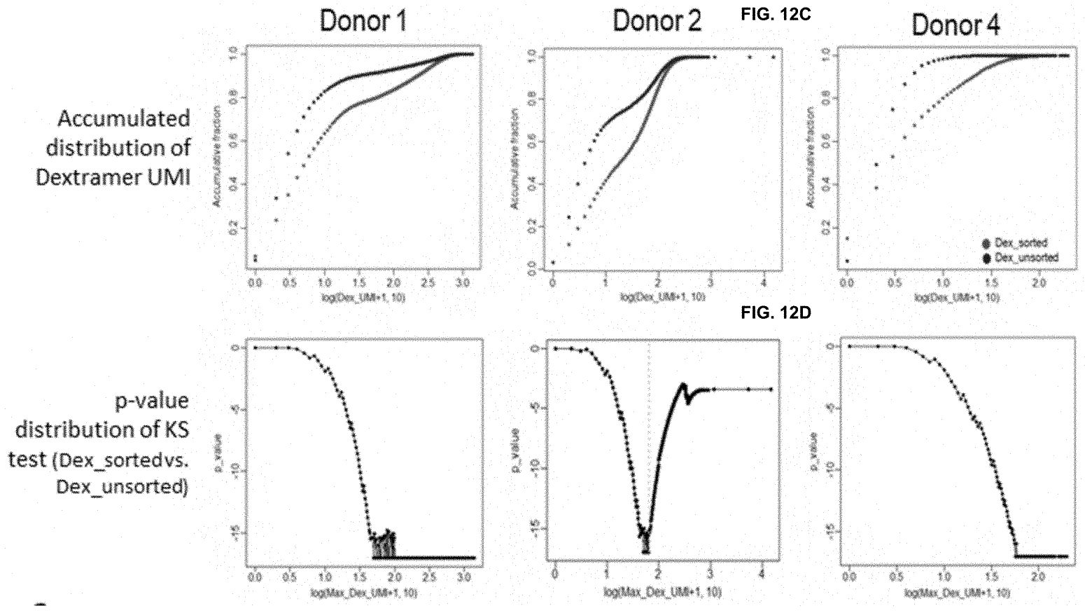

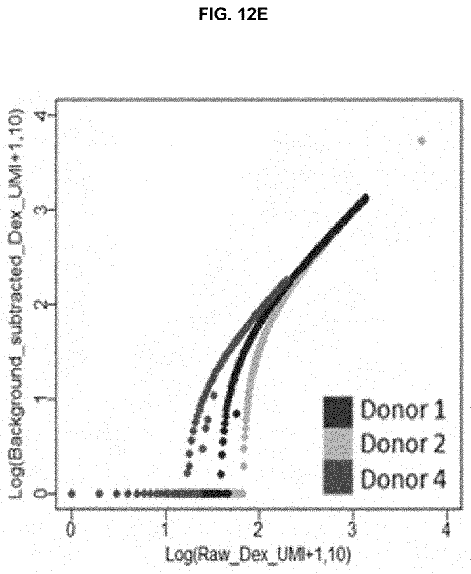

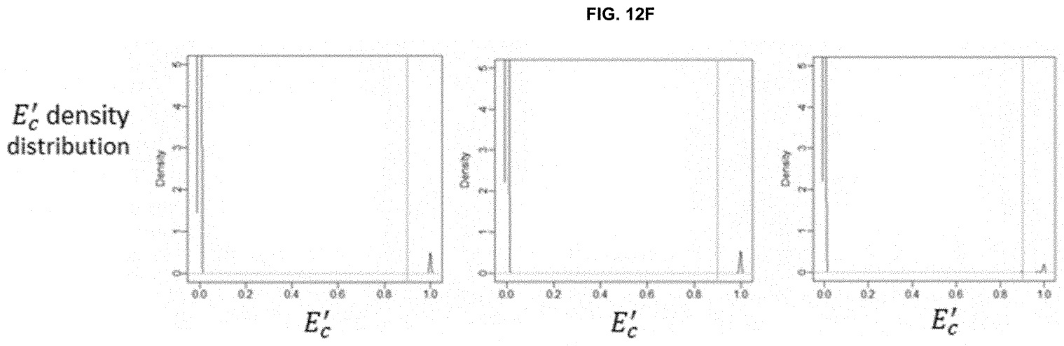

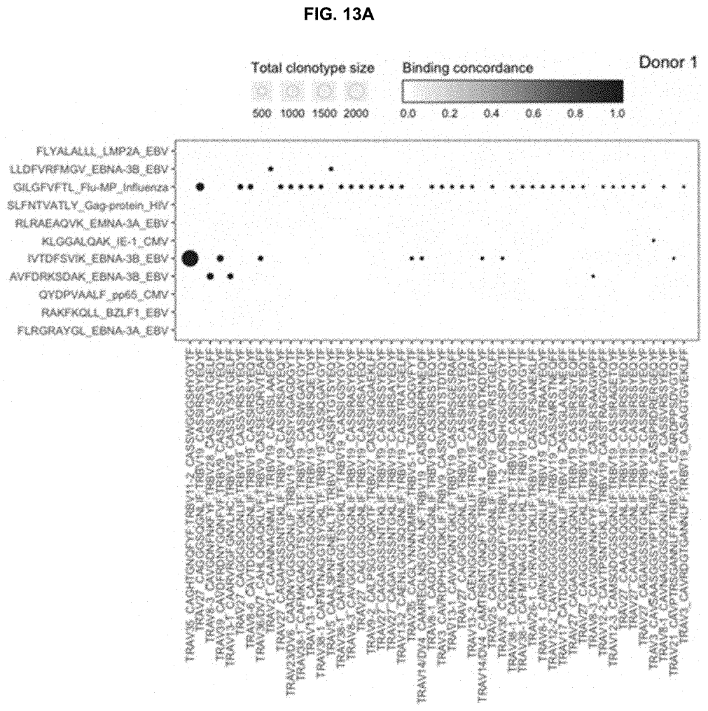

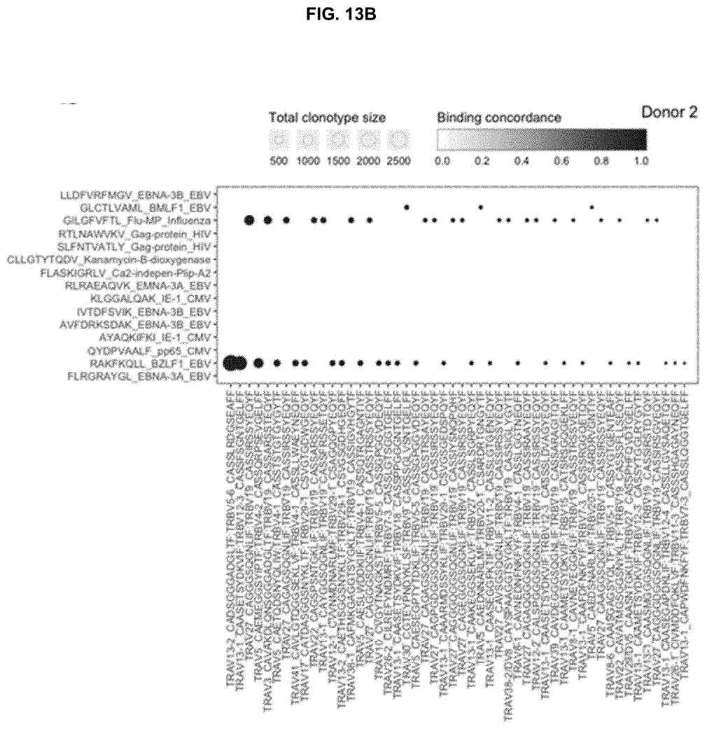

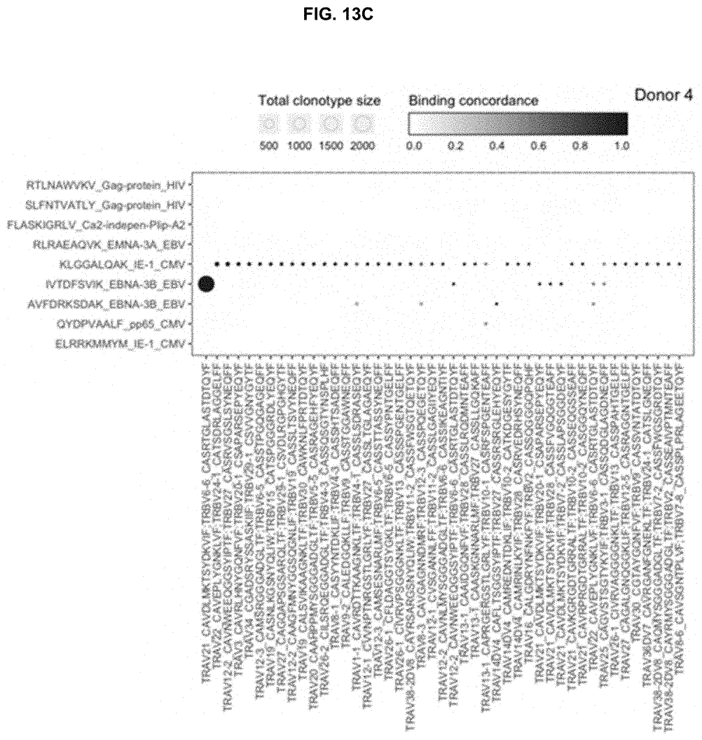

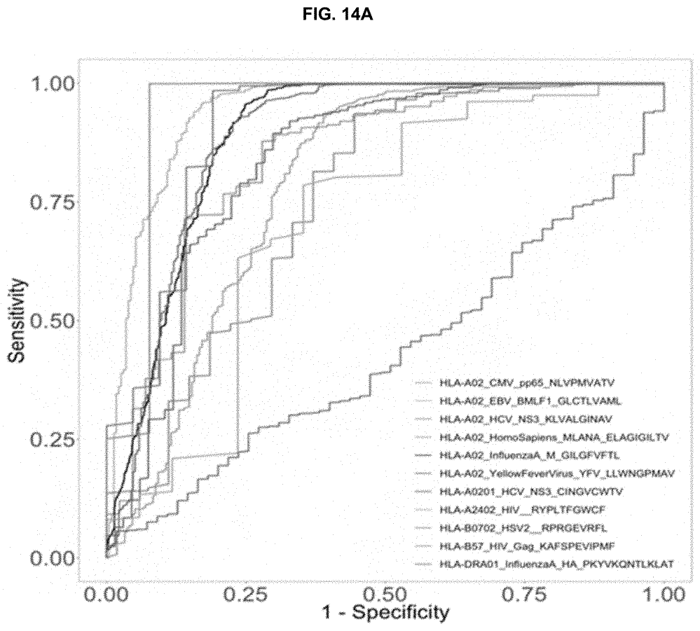

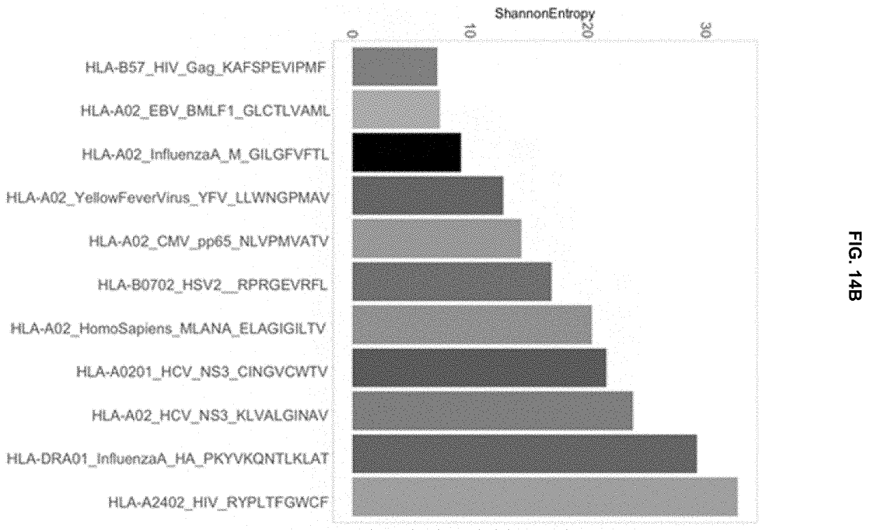

To robustly identify reliable binding events from such high-throughput TCR-pMHC binding data, ICON was developed, an Integrative COntext-specific Normalization method (FIG. 6A, FIG. 12 and Methods). The ICON data normalization process was performed in a donor-specific context by taking the multi-omics high-throughput binding data from each donor separately as input data. In brief, single cell transcriptome data was used to select good quality cells (live and singleton). Then, both negative control dextramers (n=6) and dextramer-unsorted samples were used for each donor as background controls to empirically estimate the background binding noise for each donor. Raw dextramer binding signals were subsequently corrected by subtracting the estimated background noise for each donor separately. Next, the corrected dextramer signals were normalized across cells and pMHCs to produce directly comparable dextramer binding signals. The distributions of ICON-normalized dextramer binding signals and the binding specificity of expanded T cell clones indicate that ICON significantly increased the signal-to-noise ratio of the high-throughput TCR-pMHC binding data (FIGS. 6A & 6B and FIG. 12B and FIG. 13).

ii. The TCR-pMHC Binding Events Identified from 10× Genomics High-Throughput Data.

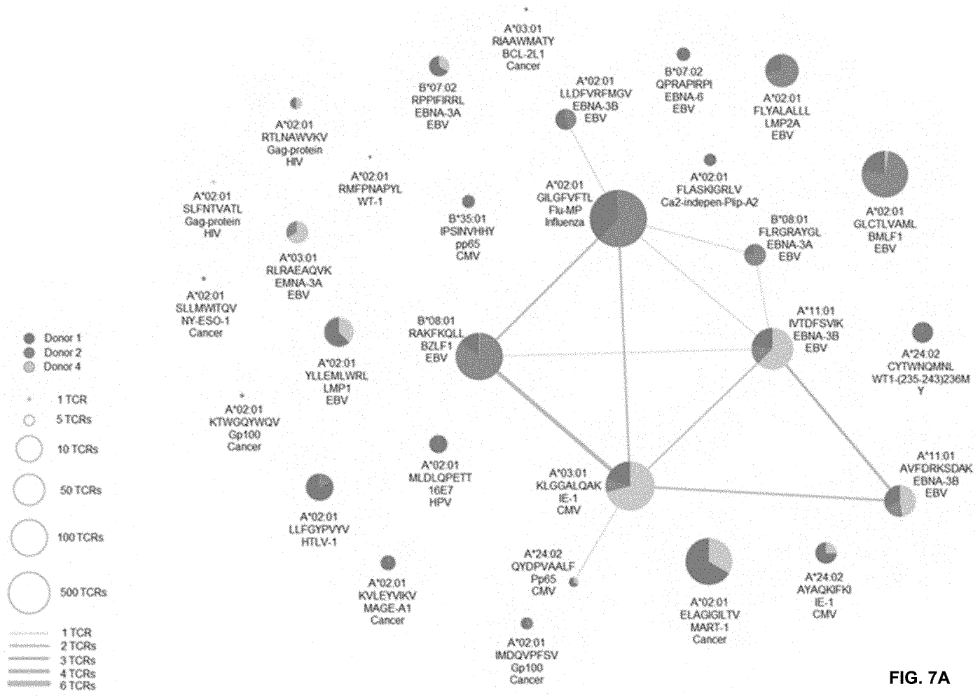

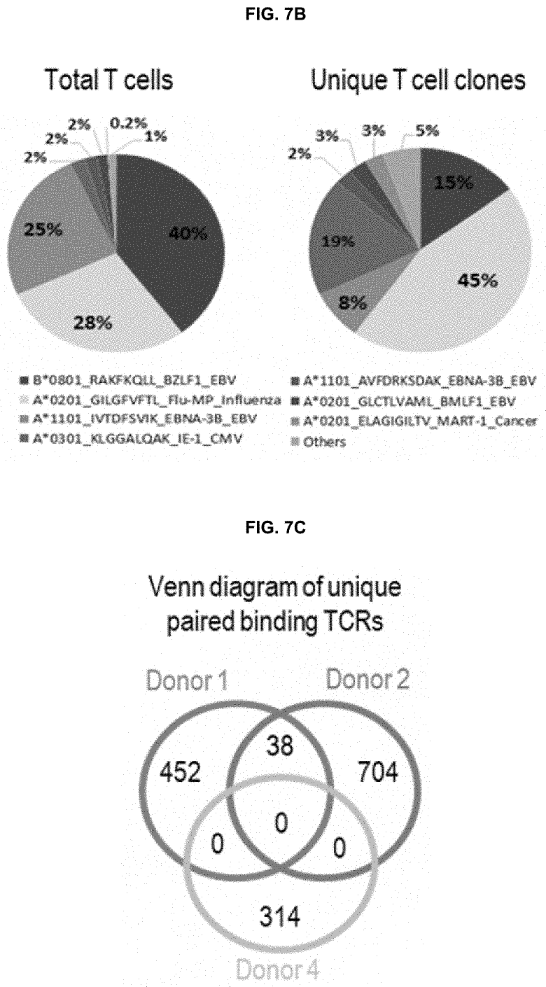

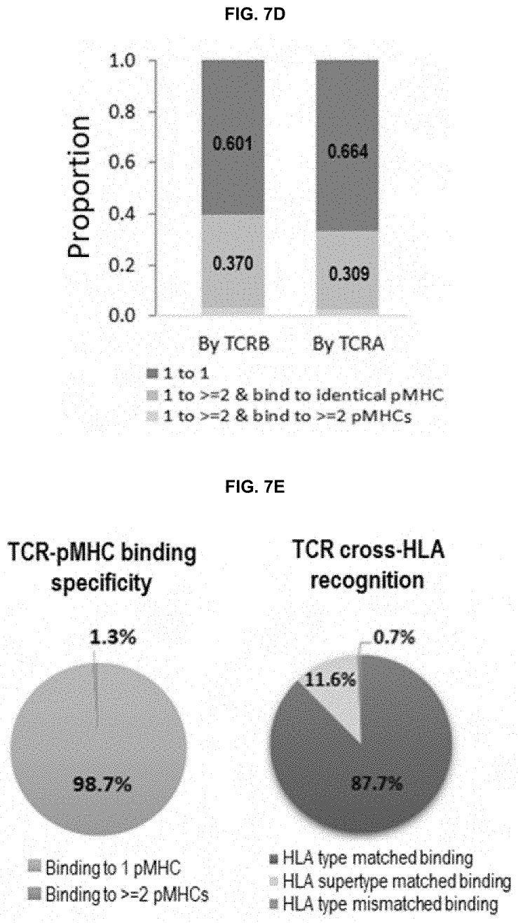

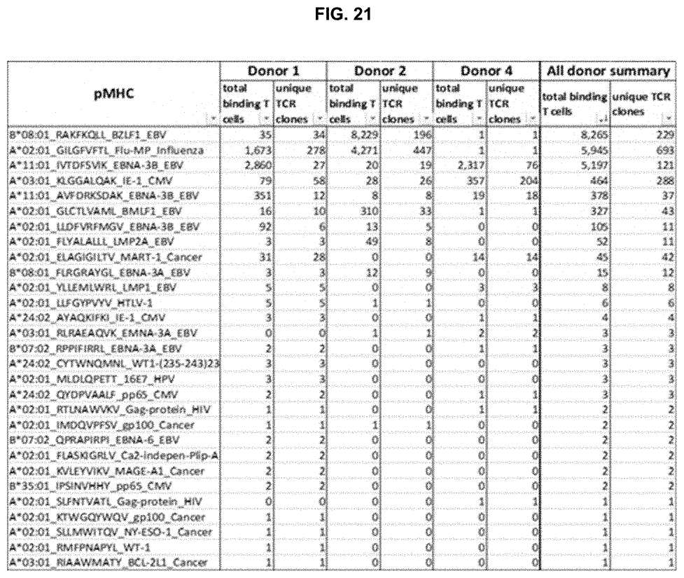

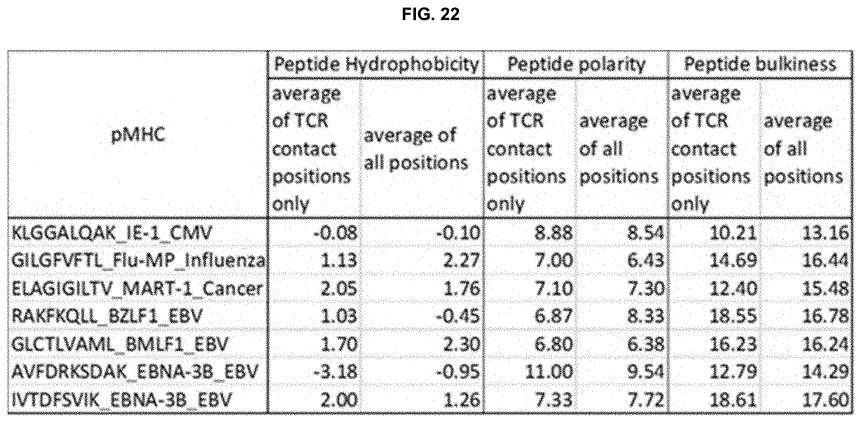

Applying ICON, a total of 20,843 CD8+ T cells were identified from 1,514 unique T cell clones that bind to 29 pMHCs from three donors (FIG. 7A, FIG. 21 and Methods). The number of unique TCR-pMHC interactions that were identified from this high-throughput dataset is comparable in size to the entirety of paired αβ TCRs in VDJdb. Among the pMHC binding TCRs, 98.9% of total TCRs (94.7% of unique TCRs) bind to seven pMHCs: B*08:01_RAKFKQLL_BZLF1_EBV, A*02:01_GILGFVFTL_Flu-MP_Influenza, A*11:01_IVTDFSVIK_EBNA-3B_EBV, A*03:01_KLGGALQAK_IE-1_CMV, A*11:01 AVFDRKSDAK_EBNA-3B_EBV, A*02:01_GLCTLVAML_BMLF1_EBV and A*02:01 ELAGIGILTV_MART-1_Cancer (FIG. 7B and FIG. 16 and FIG. 17).

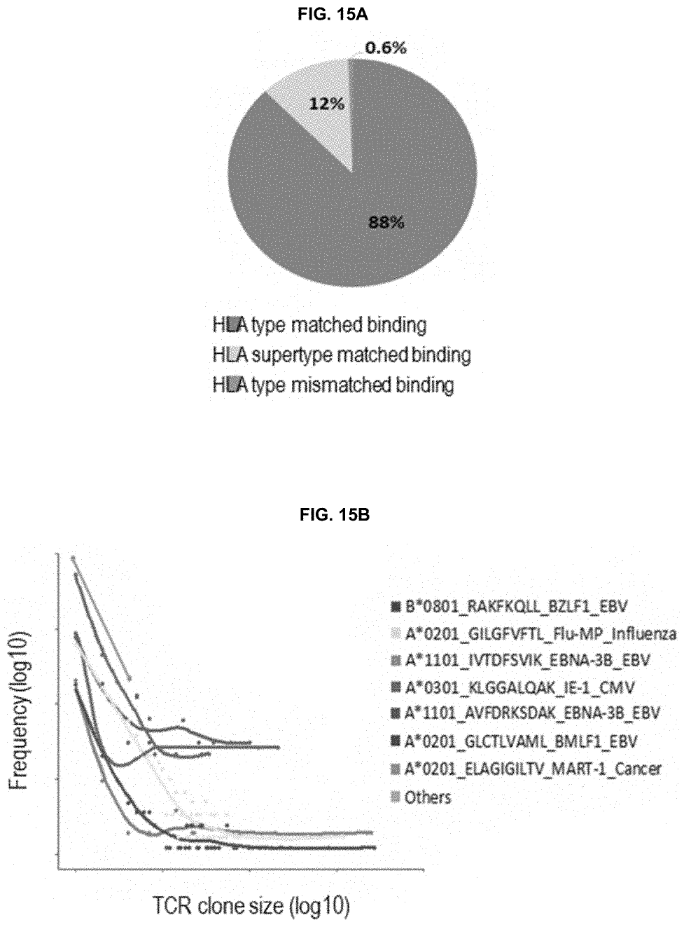

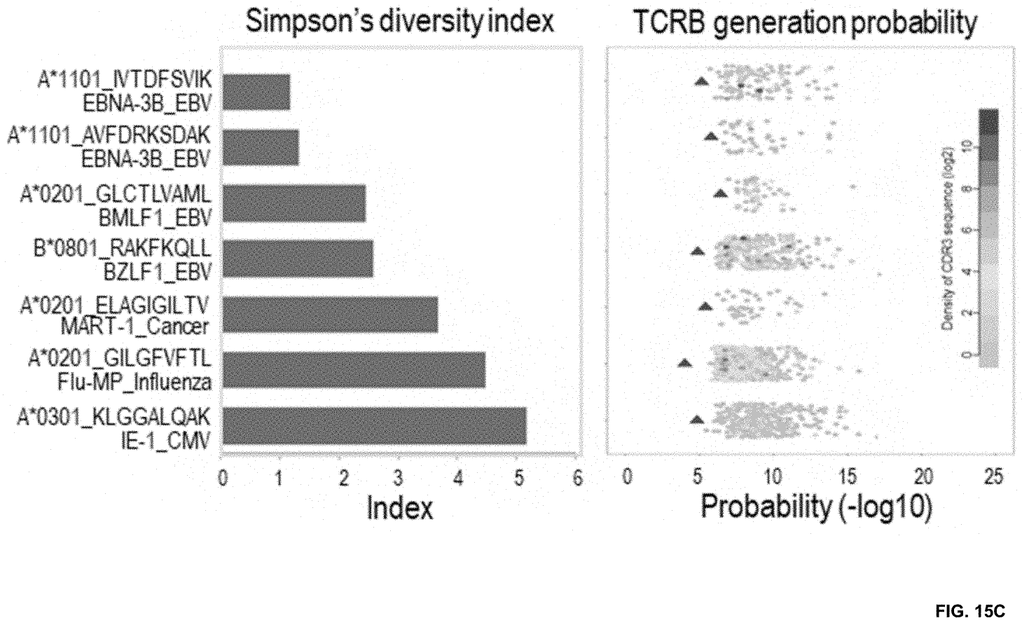

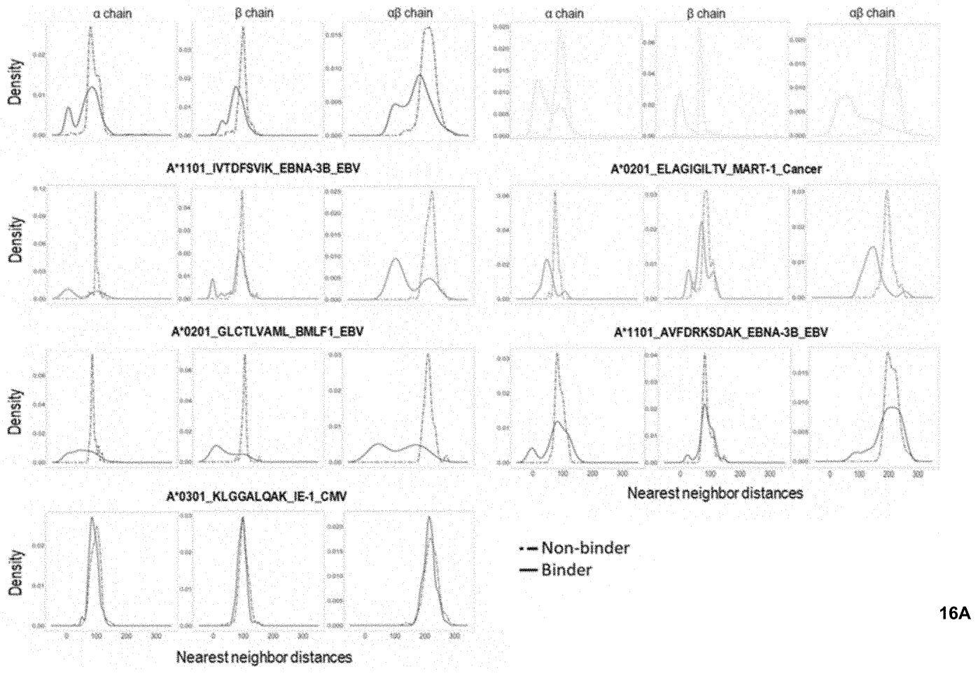

Donors 1 and 2, who possess the most common HLA haplotype (A*02:01) in the dextramer pool (FIG. 14 and FIG. 15), share a significant fraction of unique TCR-pMHC reactivities (n=38) (FIG. 7C). Donor 4 is A*02:01-negative and has a different HLA haplotype from donors 1 and 2 (FIG. 19). No shared pMHC binding TCR sequences were observed between donor 4 and the union of donors 1 and 2 (FIG. 7C), indicating that TCR-pMHC binding patterns are most likely to be HLA restricted.

Interestingly, 37% of TCRs with shared β-chains are paired with different α-chains. This rate is slightly lower (30.9%) for shared TCR α-chains. The majority of TCRs (˜92%) with shared α- or β-chains bind to the sample pMHC, but ˜8% of them recognize different pMHCs (FIG. 7D), indicating that αβ pairing information is necessary for the accurate inference of TCR functionality.

The dual specificity of TCR (specificity versus degeneracy) has been suggested as an important feature of the immune response mechanism that sufficiently distinguishes self from foreign peptides to avoid autoimmune reactivity while maintaining broad antigenic coverage. Indeed, highly specific, yet promiscuous, TCR-pMHC interactions were observed. 98.7% of unique TCRs bind to one specific pMHC and the remaining TCRs interact with 2 or 3 pMHCs (FIGS. 7E & A). Although TCRs were observed that can interact with more than one epitope, these TCR-pMHC interactions generally follow an HLA type specific pattern. Over 99.3% of binding events are HLA matched, of which 11.6% involve cross-recognition between HLA A*03-supertype family members HLA A*03:01 and A*11:01 that share similar main anchor positions of the presented peptide. However, 0.7% binding events are cross-HLA type interactions.

iii. Convolutional Neural Network (CNN) Based Classification of T Cell Antigen Specificity.

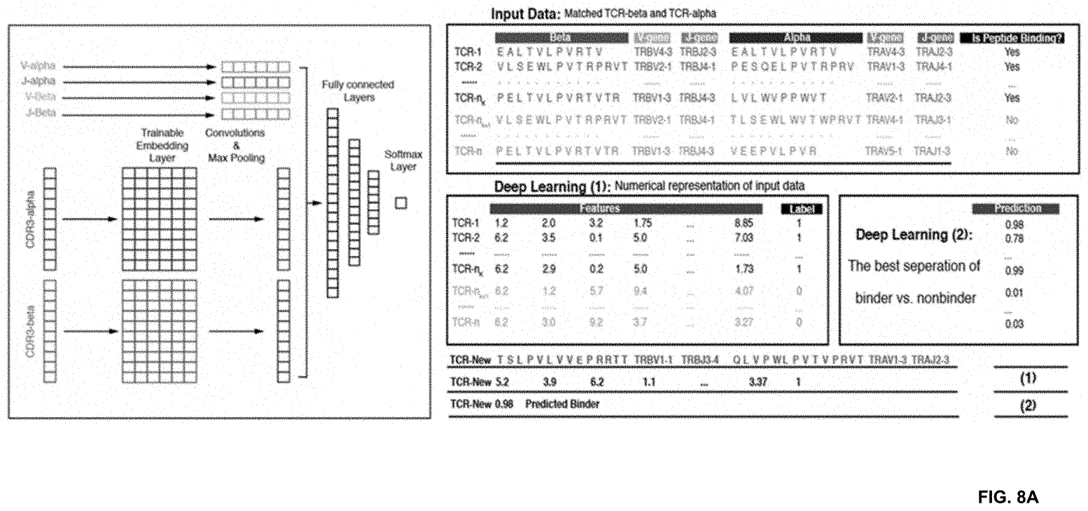

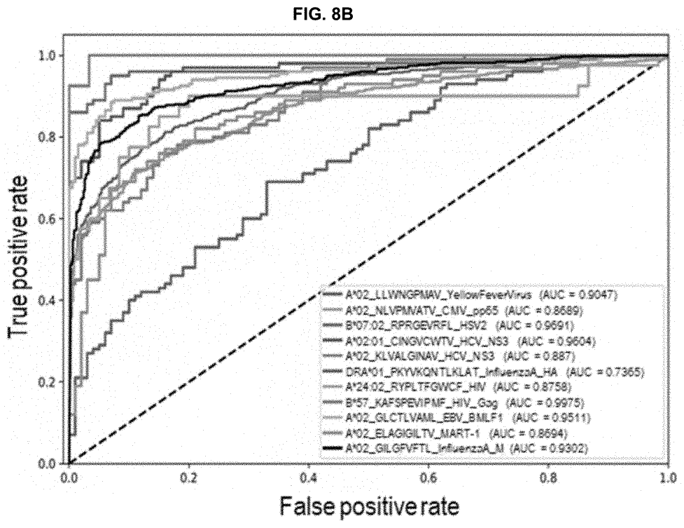

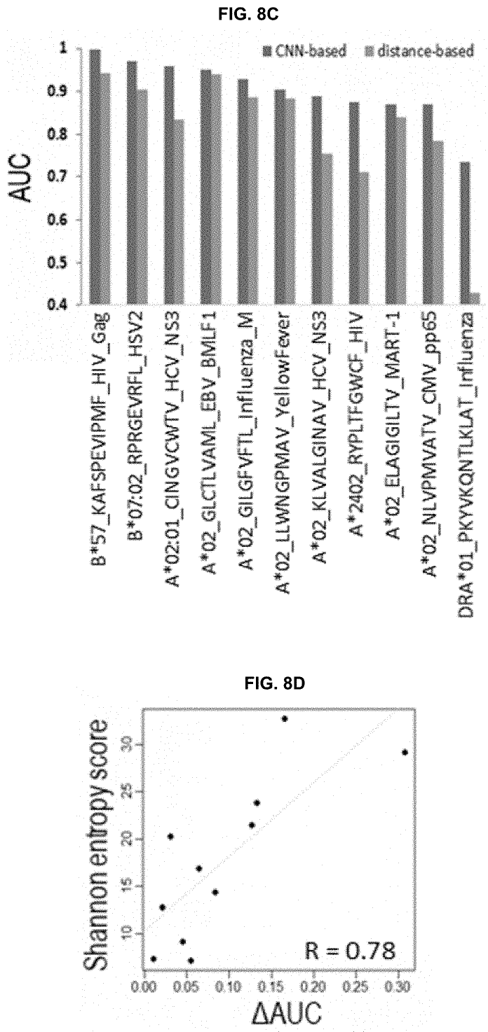

With this large, diverse TCR-pMHC binding dataset, more robust functional classifiers for computationally validating or prioritizing these binding events are desired. Recent work demonstrated that Convolutional Neural Networks (CNNs) can learn high dimensional information from TCR sequences and thus, may robustly predict TCR-pMHC binding. A CNN-based framework was adapted for validating and/or predicting TCR-pMHC binding. In brief, the paired αβ chain CDR3 amino acid sequences were encoded as well as the V and J genes of each TCR into a one-dimensional input vector. Specifically, trainable embeddings were used to encode the CDR3 amino acid sequences and the V and J gene segments were transformed into vectors. The CNN structure may comprise one convolutional feature layer and three fully connected layers leading to a final classification layer (FIG. 8A and Methods). To address the potential bias that may be introduced by having unbalanced numbers of binding and non-binding TCRs for a given pMHC, a class-weighted cost function was used for training (Methods).

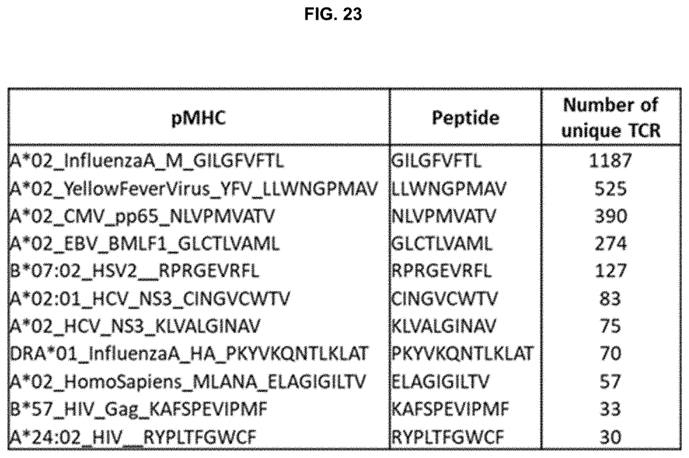

To evaluate the performance of this CNN-based model, eleven pMHC-specific binding T cell repertoires were collated generated by traditional single multimer binding and antigen re-exposure assays as a gold-standard dataset (FIG. 23). Each curated pMHC binding repertoire was split into training, validating and testing sets. The CNN-based model was able to classify the antigen binding specificity of the curated TCRs with an average Area Under the Curve (AUC) of 0.90 ((AUC)⁻=0.90) (FIG. 8B). The CNN-based classifier was compared with the TCR sequence similarity distance-based classifier. The CNN-based classifier outperforms the distance-based prediction model (FIG. 8C), particularly for highly diverse pMHC repertoires (FIG. 14). The classification performance difference between the CNN-based and the distance-based classifiers (ΔAUC) is positively correlated to the diversity of pMHC binding T cell repertoires measured by Shannon entropy (FIG. 8D).

iv. Classification of pMHC Binding Repertoires Identified from the 10× Genomics High-Throughput Data.

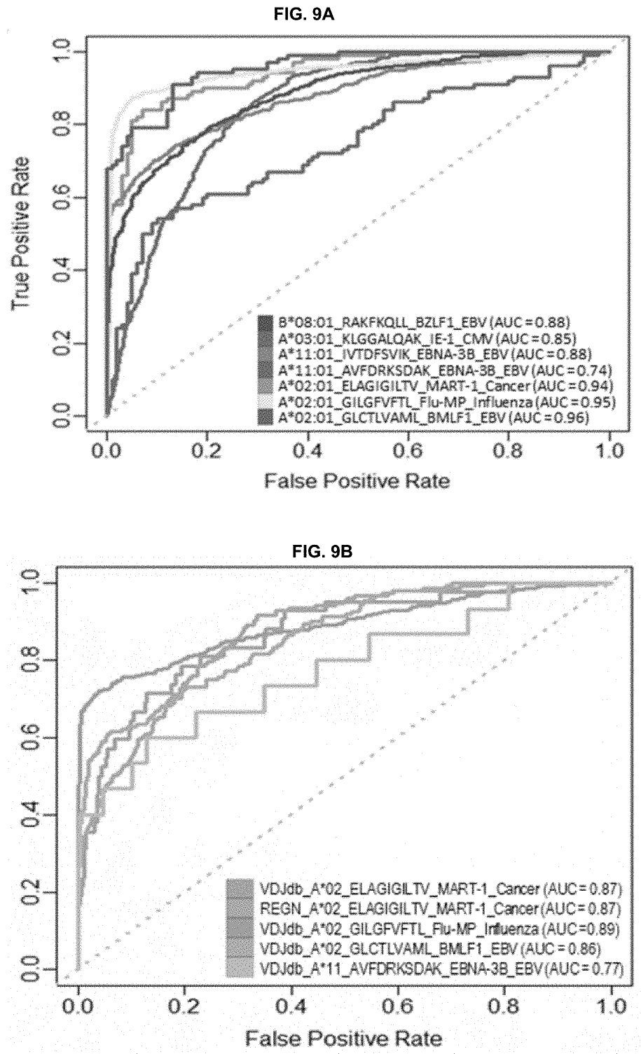

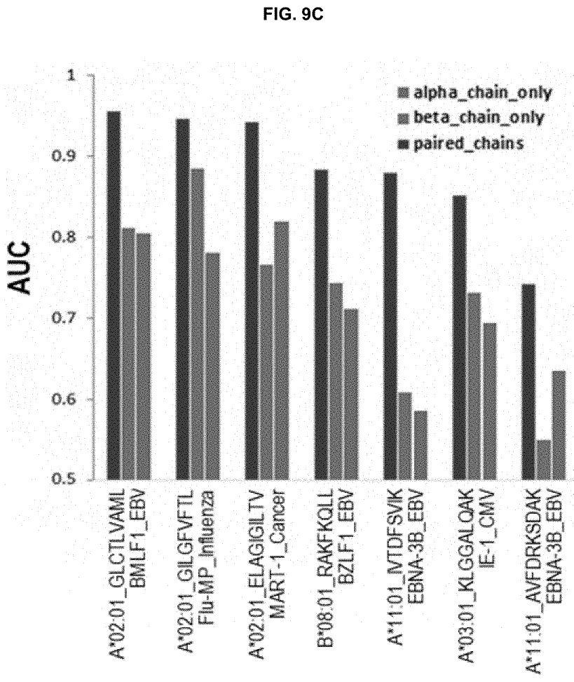

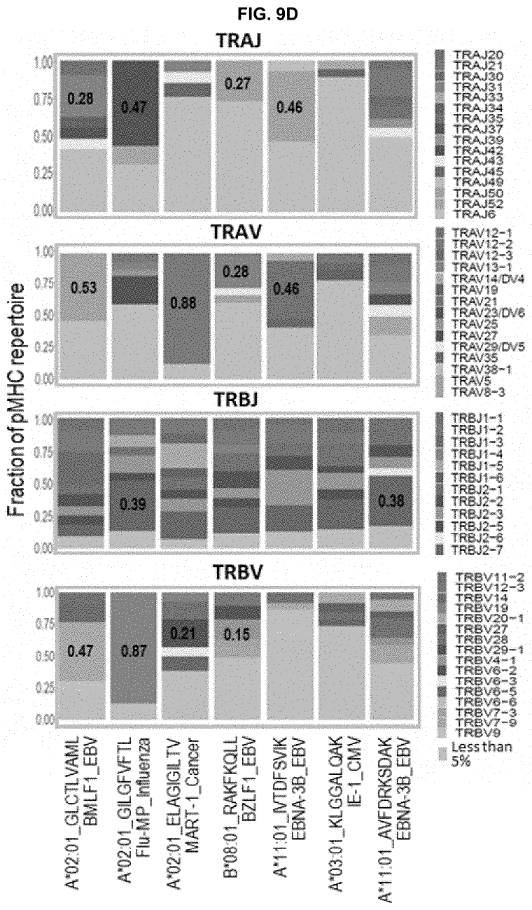

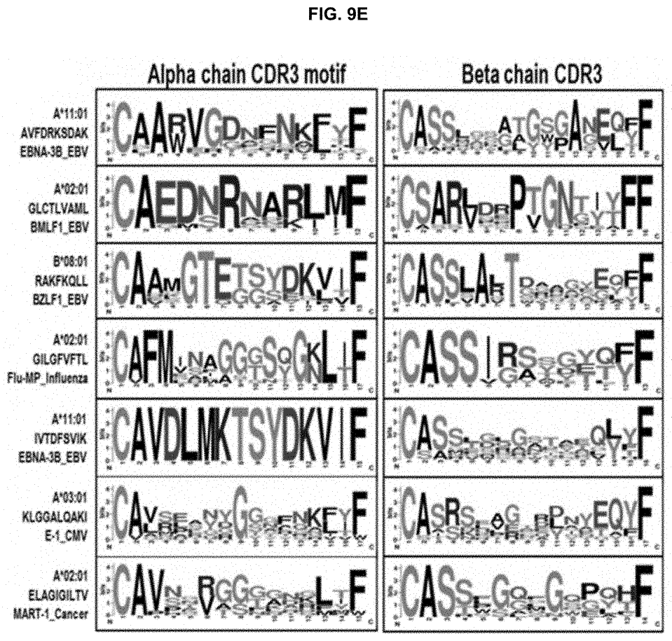

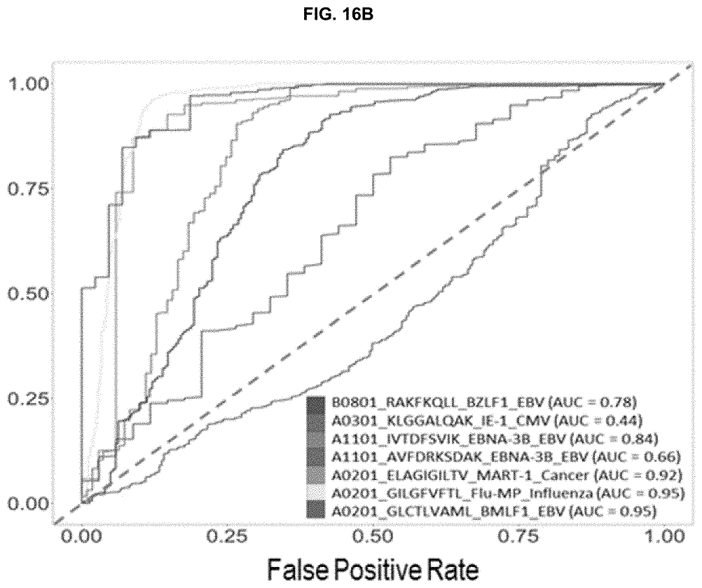

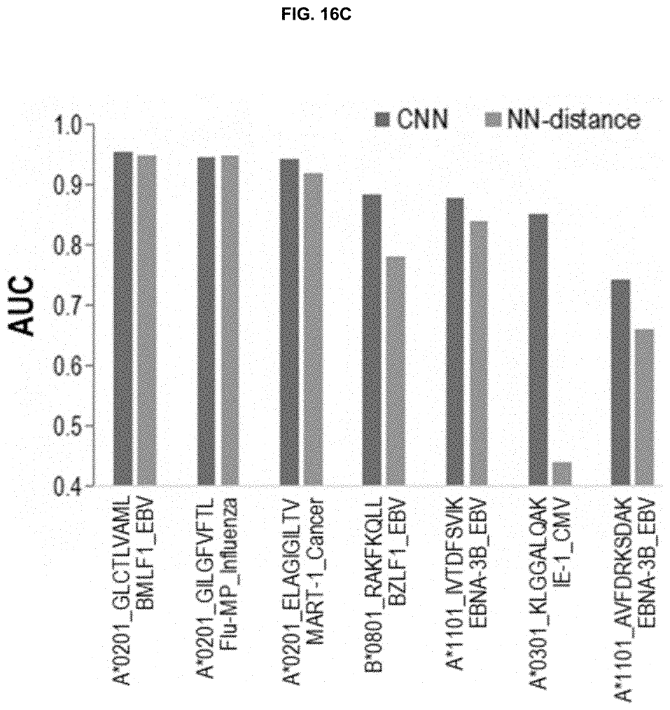

Next the CNN-based classifier was applied to the top seven pMHC binding repertoires identified from the 10× Genomics binding data (FIG. 7B and FIG. 15). The seven pMHC repertoires were classified with an average (AUC)=0.89 (FIG. 9A). On these data, as with the curated dataset, the CNN-based classifier outperforms the distance-based model (FIG. 16). To further computationally validate these binding TCRs, four pMHC repertoires (A*02:01_ELAGIGILTV_MART-1, A*02:01_GILGFVFTL_Flu-MP, A*02:01_GLCTLVAML_BMLF1_EBV, and A*11:01 AVFDRKSDAK_EBNA-3B_EBV) were used that also have binding TCRs in the curated dataset. The CNN-based classifier was trained using the four repertoires identified from the 10× Genomics dataset to predict the four curated repertoires as well as an additional A*02:01_ELAGIGILTV_MART-1 binding repertoire from an in-house independent antigen re-exposure experiment (Methods). FIG. 9B shows prediction results comparable to the high performance on the training set.

Historically, TCR β-chain sequencing was often used to infer T cell antigen binding specificity due to its higher combinatorial potential compared to the α-chain. To quantitatively evaluate the contribution of TCR α- and β-chains in predicting TCR-pMHC interaction, either the α-chain or β-chain was used in lieu of paired αβ chains as input to the CNN-based classifier. The performance with paired αβ chains is better than α- or β-chain alone with an average increase of 16% in the AUC (FIG. 9C). Unbalanced α- and β-chain contributions to the prediction of TCR-pMHC specific recognition were observed. For example, the contribution of β-chains was dominant in the A*02:01_GILGFVFTL_Flu-MP_Influenza repertoire, whereas α-chains were more important to the prediction of A*11:01_AVFDRKSDAK_EBNA-3B_EBV and A*02:01_ELAGIGILTV_MART-1_Cancer specific binders (FIG. 9C). Similarly, different levels of conservation of TCR VJ gene usage was observed between α- and β-chains of these seven pMHC repertoires (FIG. 9D). Moreover, V gene usage was generally more conserved in α-chains than in β-chains, except for dominant TRBV19 usage in the A*02:01_GILGFVFTL_Flu-MP_Influenza repertoire, which can partially explain the unbalanced classification performance between α- and β-chains. Again, these results collectively demonstrate the importance of αβ pairing for accurate inference of TCR-pMHC interactions.

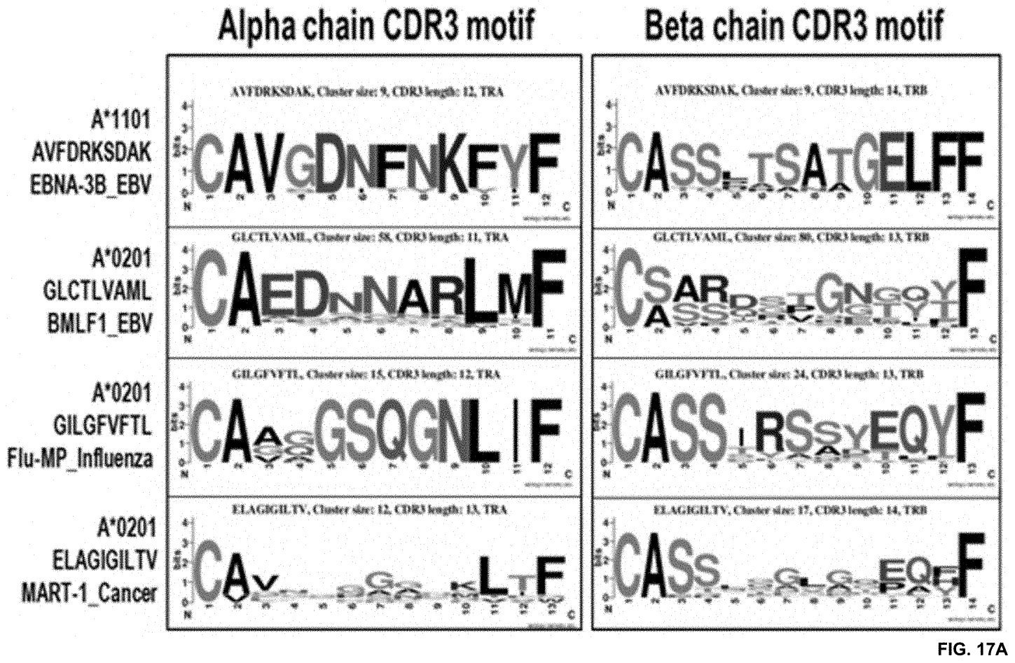

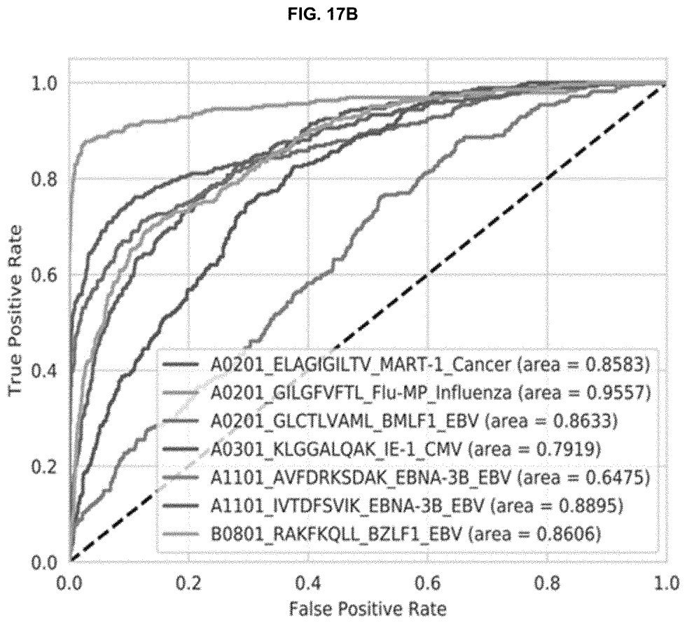

To further understand conserved TCR sequence features underlying the classification, the motif conservation of CDR3 amino acid sequences were explored from the ten most predictive TCR sequences for each of these seven pMHC repertoires (FIG. 9E). In alignment with the VJ gene usages, motif conservation is generally more evident in a-chain CDR3s than in β-chain CDR3s (FIGS. 9E and 9D). For the four pMHC repertoires for which VDJdb also has CDR3 amino acid motifs, the motifs that were identified from the 10× Genomics data are similar to those from VDJdb (FIG. 9E and FIG. 17A). Together, the results indicate that the pMHC-specific TCRs identified from the high-throughput dataset are likely reliable binding partners and the CNN-based model is able to capture key conserved TCR sequence features.

v. Immune Phenotypes of pMHC Binding CD8+ T Cells.

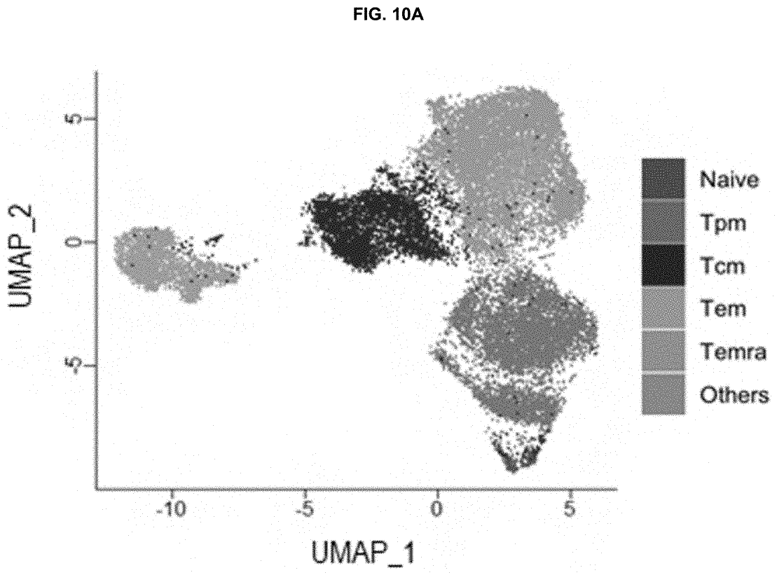

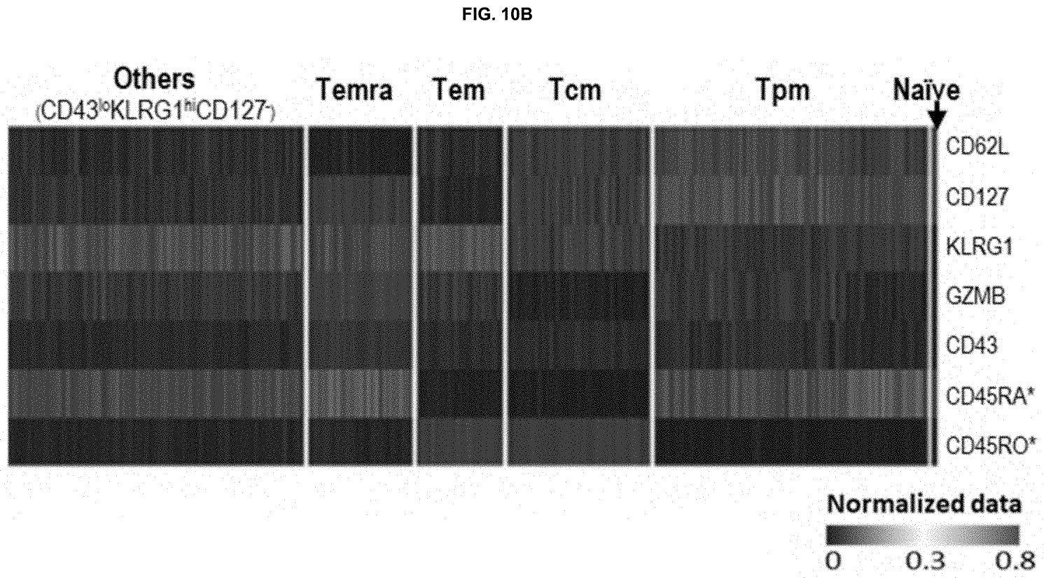

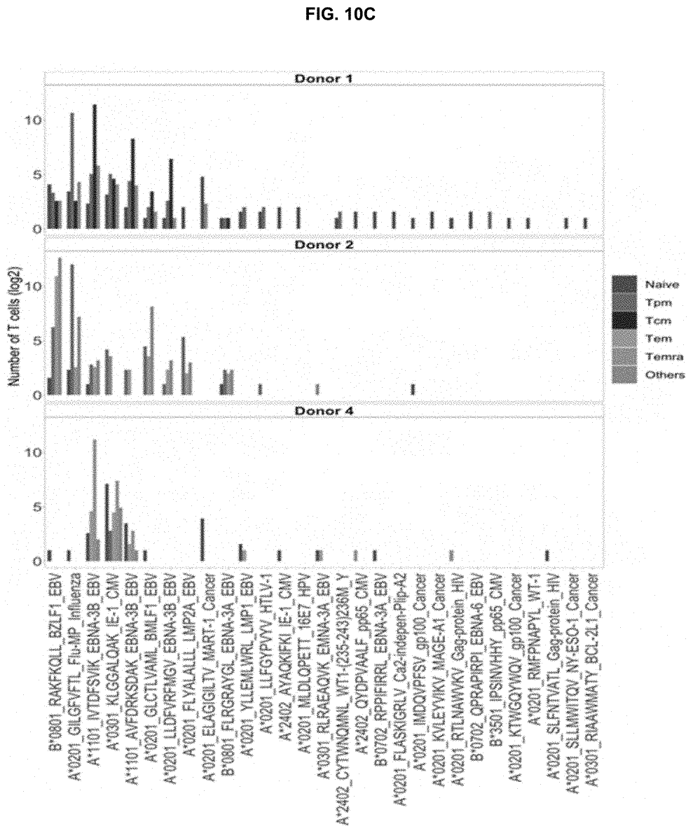

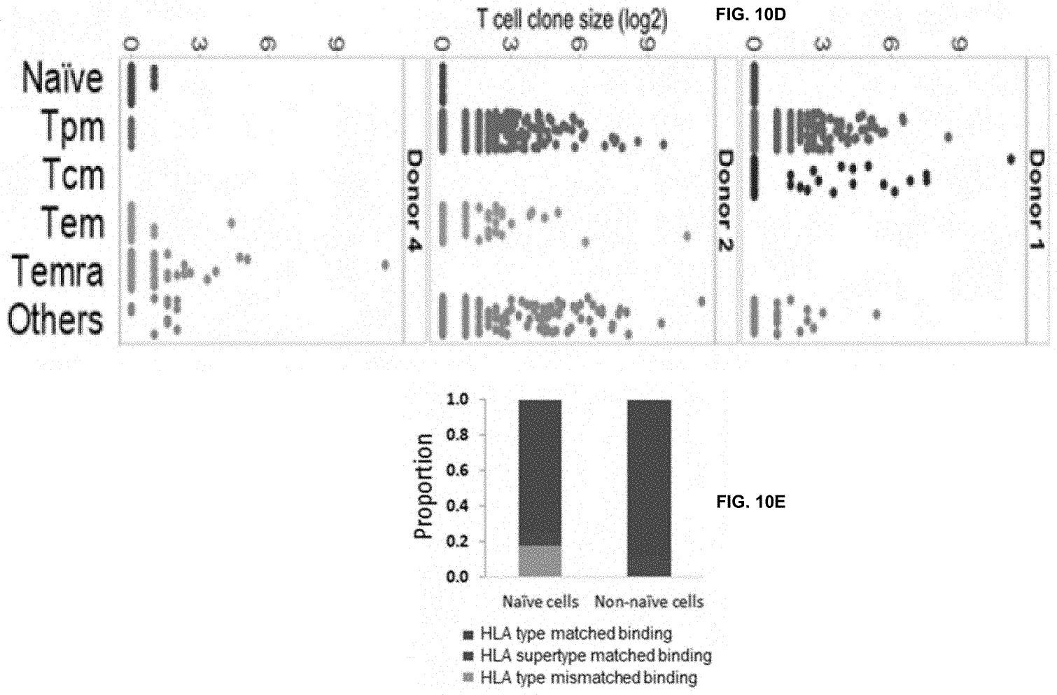

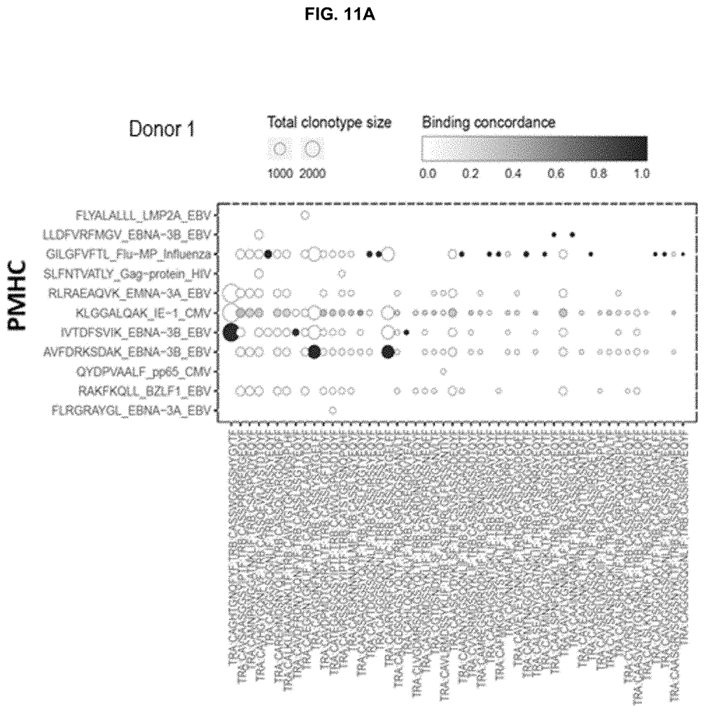

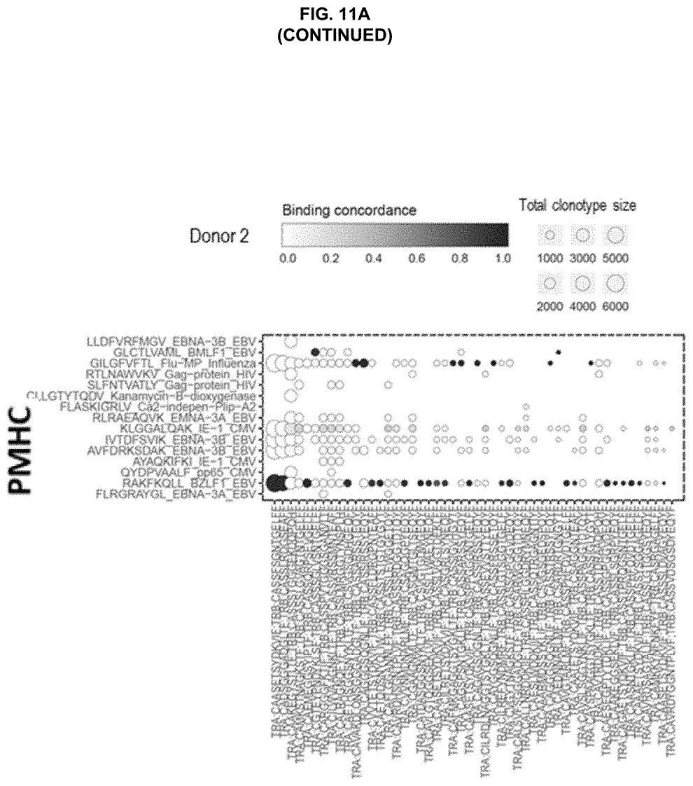

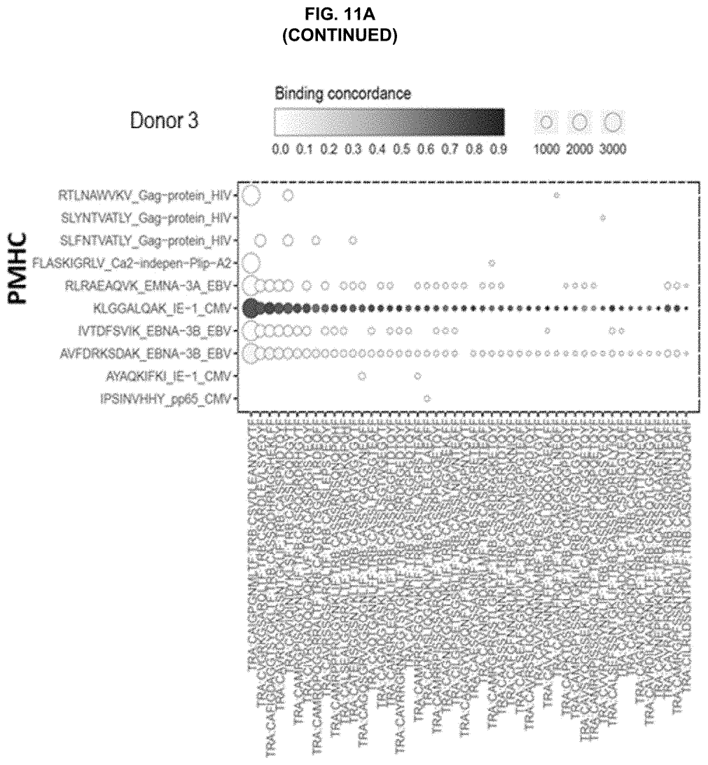

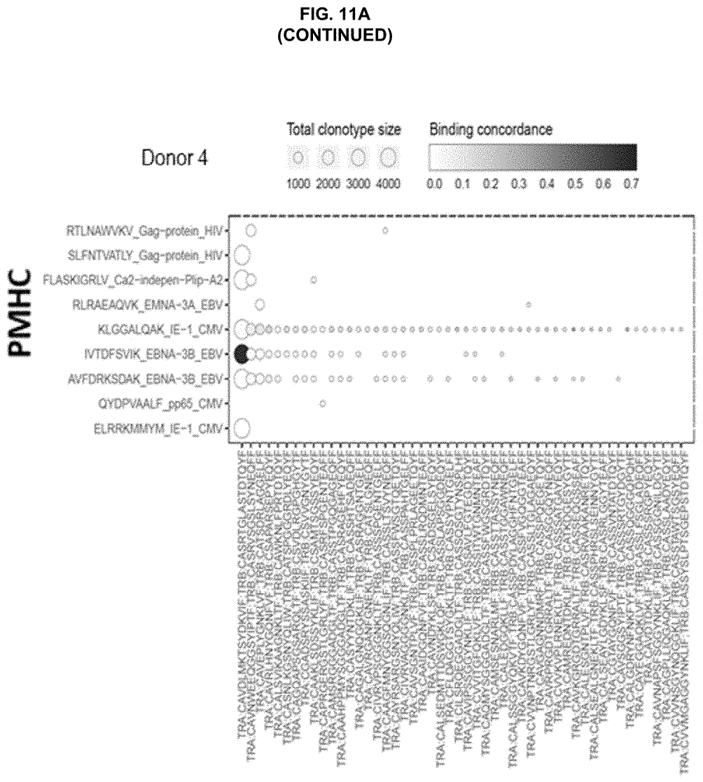

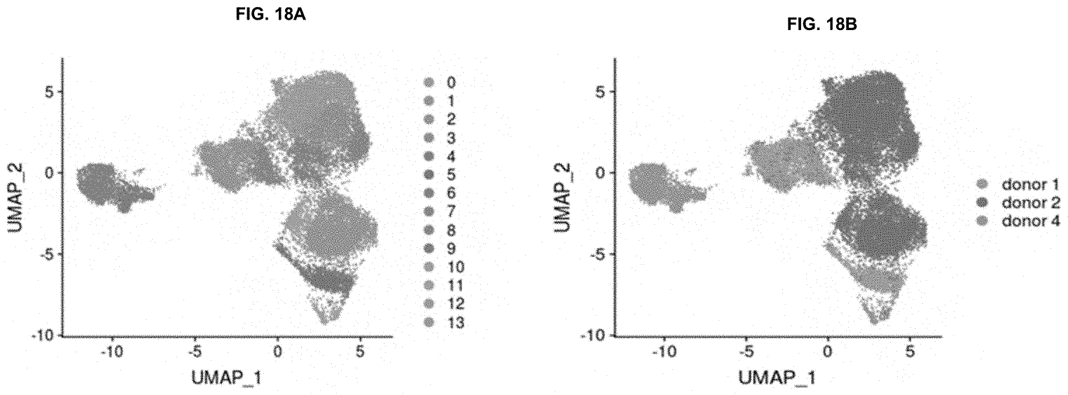

The combined information of antigen specificity and T cell phenotype has been reported to be important to clinical success of immunotherapies, such as vaccination. The multi-omics data generated by the 10× Genomics immune profiling platform enables the association of T cell antigen specificity with various T cell phenotypes. Using gene (single cell RNA-seq) and surface protein (CITE-seq) expression levels from this multi-omics dataset, pMHC binding CD8+ T cells were separated into subpopulations (Methods and FIG. 18). The identified subpopulations were then annotated according to CD8+ T cell subtype marker genes described previously 32: naïve cells (CD45RA+CD45RO−CD62LhiCD127hi), central memory cells (Tcm, CD45RA−CD45RO+ CD62L+), T effector memory cells (Tem, CD45RA−CD45RO+CD62L−), peripheral memory cells (Tpm, CD62L+CD127hi), terminally differentiated effector cells (Temra, CD45RA+CD45RO−CD127loGZMBhi) and other memory cells (CD43loKLRG1hiCD127−) (FIGS. 10A and 10B).

98.6% of pMHC binding T cells were memory cells enriched in expanded T cell clones (FIG. 10D), indicating that these T cells were selected by specific immune responses and thus are likely to be responsive and reliable binders. The majority of these memory T cells bound to common viral epitopes (e.g., influenza, EBV, CMV), and CD8+ pMHC binding T cells from each donor demonstrated different distributions of memory cell subsets. For example, donor 1 had primarily Tpm and Tcm cells, whereas donor 2 had Tem and Tpm cells, and donor 4 had mostly Temra cells (FIGS. 10C and 10D).

Although the majority of pMHC binding T cells expressed a memory phenotype, 1.3% of them were naïve cells. These naïve cells had more diverse pMHC interactions than non-naïve cells and were often bound to endogenous antigens, tumor-associated antigens (e.g. MART-1), or to antigens derived from viruses for which the donor was purportedly seronegative (e.g. HIV) (FIG. 10C and FIG. 20). Interestingly, the proportion of naïve T cells with cross-HLA type binding was significantly higher than of non-naïve cells (FIG. 10E). These results indicate that healthy donor T cell repertoires—particularly naïve cells—have the potential to respond to not-yet encountered or rare antigens and to retain cross-reactivity. Additional assays are required to assess whether these cells could mount a functional T cell response.

### 2. Discussion

A method (ICON) that can identify reliable TCR-pMHC interactions was developed by significantly increasing signal-to-background ratios in the highly multiplexed 10× Genomics TCR-pMHC binding data. Having appropriate controls (negative control dextramers and dextramer-unsorted T cell sample) is essential to accurately estimate the background noise, a factor that was found to be indispensable to reliably identify TCR-pMHC binding events. While ICON was developed on one dataset consisting of a single pool of multiplexed dextramers, this method can be generalized to query pMHC-TCR binding data from a broader range of pMHC dextramer pools as more multiplexed datasets are generated.

In this study, the robustness of this CNN-based classifier in predicting TCR-pMHC specific binding was demonstrated, indicating that this computational prediction can potentially be used to study virtually (versus experimentally) T cell antigen specific recognition. Immune monitoring of T cell antigen specific recognition has been applied to determine the immune responses against specific antigens (e.g. tumor-specific antigens and peptide vaccines) and their possible correlation with clinical outcome in patients receiving immunotherapies. However, experimentally mapping TCR sequences to antigen specificity is costly and labor intensive. With adequate training data for a particular pMHC, the classifier presented here can assign probabilities of the pMHC binding to each TCR sequence of interest without conducting binding assays. In this study, the multinomial prediction mode of this classifier (FIG. 17B) was validated, making it potentially used for selecting highly specific TCRs for safe T cell related therapies.

The results indicate that a large portion (>30%) of TCRs that bind to a specific pMHC share a single chain and differ in the second chain, emphasizing that T cell clonality must be determined by data with paired αβ chains. Additionally, 8% of these TCRs that share a single chain can bind to different pMHCs. This is in line with the predictive power of TCR antigen specificity using paired TCR chains is 16% greater than using either chain alone. Thus, single cell paired αβ chain sequencing is likely to be more powerful to accurately interrogate T cell repertoire clonality and TCR-pMHC binding specificity.

The ability to assess biologically-relevant T cell reactivities is important to interrogate and monitor immune responses to pathogens and other disease states. It was observed that the majority of the T cell reactivities recovered (98.6%) were matched with the appropriate HLA type/supertype, and further, that the phenotypes of multimer positive cells were largely restricted to memory T cell compartments, indicating that relevant memory reactivities from prior functional T cell responses are resolvable with this technology. Paired αβ TCR sequencing revealed multiple TCR sequences that were specific for individual multimers, reinforcing the broad antigenic immune responses to common viral challenges.

While a low degree of HLA mismatched reactivities were recovered, these were significantly enriched in non-expanded naïve T cells relative to memory subsets, potentially revealing antigen-specific interactions to previously unexposed targets or those that did not culminate in functional T cell responses. Additionally, it is expected that a range of TCR avidities were recovered in these experiments, which might contribute to the detection of unexpected binding patterns. Dextramers are highly multimerized and likely to detect a broader range of TCR binding avidity than traditional tetramer reagents. Furthermore, a range of fluorescent dextramer intensities were sorted in the multimer-positive gating, so even low-frequency, lower-avidity TCR interactions were captured in this highly-sensitive single cell assay.

3. Methods

i. The 10× Genomics Single Cell Immune Profiling Datasets

10× Genomics data used for this study were downloaded from: support.10×genomics.com/single-cell-vdj/datasets

ii. Single-Cell RNA-Seq Data QC

CD8+ cells from each donor were selected for the downstream analysis by the following criteria: number of RNA features <=2500 and >200 genes detected per cell, and mitochondria percentage is less than 40 percent of the total UMI (unique molecular identifier) counts.

iii. Classification pMHC Binding T Cell

Seurat V3 single-cell sequencing analysis R package33, 34 was used for the classification analysis based on single cell RNA-seq data. Since the significant enrichment of TCR VJ gene usages was observed in identified pMHC binding T cells, the TCR genes were taken out from the classification. So, cell clusters will not be dominated by their shared VJ gene usage. Then, all other gene expression of identified binding T cells was normalized and scaled using Seurat V3 default parameters. PCA was run on normalized and transformed UMI counts on variably expressed genes. Top 10 PCs were used for the cell classification. UMAP was used for classification visualization (FIG. 17).

iv. Generating CDR3 Motifs from the Most Predictive pMHC Binding TCR Pairs

The CDR3 amino acid sequences of α and β chains from the ten most predictive TCRs were aligned using COBALT (www.ncbi.nlm.nih.gov/tools/cobalt/cobalt.cgi). Aligned CDR3 amino acid sequences were input into WebLogo35 with default parameters to generate motifs.

v. Curation of Reported pMHC Specific Binding Paired TCRs

Raw files were downloaded from VDJdb28 (vdjdb.cdr3.net/) and The Pathology-associated TCR database36 (friedmanlab.weizmann.ac.il/McPAS-TCR/). The data was processed to get pMHC TCR binding following the following criteria: for VDJdb, paired α- or β-chain CDR3 amino acid sequences were required for each “complex.id”; TCRs annotated with “source” were removed from 10× genomics; data was filtered for “Species”=“Human”. For McPAS-TCR, known “Epitope.ID” were required in the full data and having “CDR3.alpha.aa” and “CDR3.beta.aa”; Similarly for VDJdb, Human TCRs were filtered for.

vi. Normalization of TCR-pMHC Binding Data

An Integrative COntext-specific Normalization (ICON) method was developed. It takes the multi-omics single cell sequencing data generated from the 10× Genomics Immune Map platform as input data and performs TCR-pMHC binding specificity data normalization to identify reliable binding events. The multi-omics dataset includes single cell RNA-seq, paired αβ chain single cell TCR-seq, dCODE-Dextramer-seq and cell surface protein expression sequencing—also named CITE-seq (cellular indexing of transcriptomes and epitopes by sequencing). ICON includes the following major steps (FIG. 6A and FIG. 12):

Single cell RNA-seq based filtering of low-quality cells. It filters out low quality cells such as doublets and dead cells. The cells with an unexpectedly high number of genes for T cells detected (e.g. >2500 genes per cell) were categorized as doublets and cells with a high fraction of mitochondrial gene expression (e.g. ratio of mitochondrial gene expression UMIs to the total gene expression UMIs>0.4) or too few numbers of genes detected (<200 genes per cell) were classified as dead cells. (FIG. 12A).

Single cell dCODE-Dextramer-seq based background adjustment. There are two types of background noise controls that were designed for the dextramer binding assays and were used in the analysis: one is negative control dextramers (n=6) from dextramer stained and sorted CD8+ T cells (NC_dex, denoted as nc), and the other is dextramer stained CD8+ T cells without sorting on dextramer (Dex_unsorted, denoted as du). To inspect signal and noise distributions, the maximum dextramer signal in UMI (Unique Molecular Identifier) of each cell was chosen to represent the best binding of each cell. Specifically, the non-specific dextramer binding signal of a cell is represented as Max (nc₁, . . . , nc₆), the maximum dextramer signal of the 6 negative control dextramers included the dextramer pool. The dextramer binding signal of a cell from a dextramer stained and sorted sample (Dex_sorted, denoted as ds) is represented as Max(ds₁, . . . , ds₄₄), the maximum dextramer signal in UMI of the 44 testing dextramers. Similarly, the dextramer binding signal of a cell from a Dex_unsorted sample is represented as Max(du, . . . , du₄₄). The distributions of these three types of dextramer signals before ICON process are shown in FIG. 12B upper panel. P_{99.9} (absolute outliers of negative dextramer controls were excluded) of the non-specific dextramer binding signals in UMI was chosen for each donor as non-specific dextramer binding cutoff.

To estimate the potential noise introduced by the cell sorting process, the accumulative distributions of dextramer binding signals were compared between Dex_sorted and Dex_unsorted samples to determine the cutoff for dextramer sorting efficiency (FIG. 12C). For each donor, the Kolmogorov-Smirnov test (KS test) p-values were calculated by comparing the accumulative curves of dextramer sorted and dextramer unsorted samples using each data point (dextramer UMI) as a sliding window. S-shape decrease p-value curves indicate the enrichment of dextramer binding signals in dextramer sorted samples comparing to dextramer unsorted samples, while the V-shaped curve suggests a loose cell sorting gate (FIG. 12D). The dextramer UMI which defines the largest difference of dextramer binding signals between Dex_sorted and Dex_unsorted (argmax D_(s,u)) was used as the threshold for estimating dextramer sorting efficiency for the V-shaped sample. Finally, the background noise of dextramer sorted samples was defined as:

d=Max(P_{99.9},argmax Ds,u)

The dextramer signals (UMI) for each 44 testing dextramer of sorted cells was corrected by subtracting the estimated background (FIG. 12E):

E_{c}=Eₛ−d

Then, a cell-wise normalization was conducted based on Log-Rank distribution for each cell. A pMHC-wise normalization was performed to make the dextramer binding signal comparable to each other. The adjusted dextramer binding signals of sorted cells E_c were normalized across 44 testing dextramers, then across all cells as the following equation. E_c\hat{ }′>=0.9 was chosen empirically as the cutoff for pMHC specific binders (FIG. 12F).

$${E}_{c}^{′} = \frac{log ( {E}_{c}^{ij} , 10 )}{{\sum }_{j = 1}^{j = N} log ( {E}_{c}^{ij} , 10 )}$$

$${E}^{*} = \frac{{{E}^{′}}_{c}^{ij} - {\overline{{E}^{′}}}_{c}^{j}}{σ ( {E}^{{′}_{c}^{{{ }^{′}}^{′} j}} )}$$

Selecting T cells with single paired αβ chains based on single cell TCR-seq. T cells were removed that have only α-chain, only β-chain, and multiple α- or β-chains. Only the T cells with the single paired αβ chains were used in this study.

The ICON normalization process was performed for each donor separately.

vii. Antigen-Specific T Cell Expansion and Antigen Re-Exposure to Identify MART-1 Binding T Cells

Peripheral blood mononuclear cells (PBMC) from HLA A*02:01 individuals were isolated by Ficoll-Paque Plus gradient isolation. PBMC were seeded to culture plates in T cell media (CellGenix dendritic cell media, cat #20801-0500+5% human serum AB (Sigma, cat #H3667))+1% penicillin/streptomycin/L-glutamine (ThermoFisher, cat #10378-016), the T cell supporting cytokines IL-7 and IL-15 at 5 ng/ml (CellGenix, cat #1410-050 and 1413-050, respectively), and IL-2 at 10 U/ml (Peprotech, cat #200-0), and the A*02:01-restricted MART-1 epitope ELAGIGILTV at 10 ug/ml (Genscript). Cultures were fed with fresh media and cytokines every two days for one week. On day seven of culture, cells were stained with the fluorescently-tagged dextramer HLA-A*02:01 MART-1 ELAGIGILT (Immudex, cat #WB2162-PE) to assess antigen specific CD8+ T cell expansion by flow cytometry. For antigen re-exposure assays, the peptide was added to T cell expansion cultures after 7 days of expansion. Twenty-four hours following re-stimulation, cells were collected and stained with fluorescently-labeled antibodies for CD3 (BD Biosciences, cat #612750), CD8 (BD Biosciences, cat #612889), CD69 (BD Biosciences, cat #564364), CCR7 (Biolegend, cat #353218), CD45RO (Biolegend, cat #304238), CD137 (Biolegend, cat #309828), and CD25 (Biolegend, cat #356104). Utilizing an Astrios cell sorter (Beckman Coulter), fluorescence activated cell sorting (FACS) gating on forward scatter plot, side scatter plot, and fluorescent channels was set to select live cells while excluding debris and doublets. a 100 μm nozzle was used to sort single CD3+CD8+CD45RO+CD137+ cells for further processing.

Sorted cells were then loaded onto a Chromium Single Cell 5′ Chip (10× Genomics, cat #) and processed them through the Chromium Controller to generate GEMs (Gel Beads in Emulsion). RNA-Seq libraries were prepared with the Chromium Single Cell 5′ Library & Gel Bead Kit (10× Genomics, cat #) following the manufacturer's protocol.

viii. Regeneron Oligo-Tagged Dextramer Staining and Sorting for 10× Genomics Donor 3 and Donor 4

10× Genomics kindly provided cryopreserved donor 3 and donor 4 PBMCs for use in reassessing CD8+ T cell dextramer binding ability. CD8+ T cells were enriched using Miltenyi CD8+ T cell negative enrichment (Mitenyi). The cells were then incubated for 45 minutes with benzonase (Millipore) and dasatinib (Axon) before being stained with oligo-tagged dextramer pools (Immudex, FIG. 21) for 30 minutes at room temperature. Cells were then stained with fluorescently labeled for CD3 (BD Biosciences, cat #612750), CD4 (BD Biosciences, cat #563919, CD8 (BD Biosciences, cat #612889), CCR7 (Biolegend, cat #353218), and CD45RO (Biolegend, cat #304238) and CITE-seq antibodies for an additional 30 minutes on ice. Utilizing an Astrios cell sorter (Beckman Coulter), fluorescence activated cell sorting (FACS) gating on forward scatter plot, side scatter plot, and fluorescent channels was set to select live cells while excluding debris and doublets. A 100 μm nozzle was used to sort single CD3+CD8+dextramer+ cells for further processing (FIG. 11).

TCR sequence similarity distance-based classification recently reported a weighted hamming distance-based method, TCRdist, to predict TCR-pMHC binding specificity based on the sequence space of TCR CDR regions guided by structural information on pMHC binding. Nearest-neighbor (NN) distance (the average TCRdist between a receptor and its nearest-neighbor receptors within the repertoire) was further calculated to measure receptor density within repertoires. For each pMHC repertoire, binders were defined to be TCRs that bind to the given pMHC. NN-distances were calculated between each binding TCR and each set of pMHC binders with the given TCR removed. The NN distances were separated based on the known specificity of each TCR. Receiver operating characteristic (ROC) curves and area under the ROC curve (AUC) were calculated for the binary classifier of each pMHC using the plotROC R package38. In brief, ROC curves were generated by calculating sensitivity and specificity at several NN distance thresholds for each classifier—classifying TCRs as binding to a given pMHC if their NN distance falls below the given threshold.

ix. CNN-Based Classification

The weighted binary classifier was adapted based on a deep learning framework, which includes three major steps with adjustments made to accommodate the specific needs.

x. Input Data Formatting

TCR sequencing files were collected as a raw csv formatted file from 10× Genomics. Sequencing files were parsed to take the amino acid sequence of the CDR3 after removing unproductive sequences. Clones with different nucleotide sequences but the same matched amino acid sequence from CDR3s and the V, D, J genes were aggregated together under one TCR. Thus, each TCR record used here includes single paired α and β TCR amino acids sequences of CDR3, V, and J genes. For the model running with α-chain only, TCRB-CDR3 amino acid sequences, β-chain genes were removed from the input. Similar removal was done for the β-chain only model.

xi. Data Transformations

Each TCR-CDR3 amino acid sequence was encoded into numbers to represent the 20 possible amino acids. Only sequences that comply with IUPAC (International Union of Pure and Applied Chemistry) amino acids were kept. 0-padding was applied to a maximum length of 40 for TCRs of different length. A trainable embedded layer was used to further extract features from the amino acid sequences. The V and J genes were one-hot encoded to provide a categorical and discrete representation of the gene names in numerical space. The encoded sequences and gene names were concatenated together to represent one TCR record. This data transformation process was applied before training all networks.

xii. Single TCR Sequence Classifier

This method was adapted, where they provided a general conventional neural network architecture to train TCR and focused on sample or repertoire level prediction. Optimizing single TCR sequence prediction was focused on. To achieve this, T cell clone size was removed from the input data. In addition, a single translationally invariant layer was applied to the sequence followed by three fully connected convolutional layers to a final output layer. The network was trained using an Adam Optimizer (learning rate=0.001) to minimize the cross-entropy loss between the soft-maxed-logits and the one-hot encoded representation of the discrete categorical outputs of the network. This approach was modified by using a biologically meaningful kernel size of 439 to capture potential motifs. To account for the unbalanced class representation in the training data, weighted cross-entropy loss function was applied using the following formula:

Σᵢ₌₀ⁿw_{c}*(ŷᵢ−yᵢ),w_{c}=n/n_{c}

w_{c} is the weights computed using the inverted frequency of TCR sequences for each class. C represents one class; n_{c} is the total TCR in one class; n is total number of TCRs; ŷᵢ, yᵢ represent predicted and actual class for each TCR sequence.

A Monte-Carlo Cross Validation (MCCV) training was conducted by holding a certain number of TCRs for validation and testing, respectively. The validation group of sequences was used to implement an early stopping algorithm. Here, 20 iterations were taken of Monte-Carlo sampling. A Receiver Operating Characteristic (ROC) curve for the sequence classifier was computed based on the testing set after averaging on all MCCV predictions.

### B. Example 2

1. Results

i. Identification of pMHC Specific Binding TCRs from High-Throughput Binding Data

10× Genomics recently generated an expansive, publicly available TCR-pMHC binding dataset. In their initial report, the binding profile of over 150,000 CD8+ T cells from four HLA haplotyped healthy donors (Table 1, donors 1 to 4) was assessed across 44 pMHC dextramers using a single cell-based immune profiling platform Immune Map to directly detect antigen binding to T cells, while simultaneously sequencing T cell αβ chain pairs and transcriptomes (FIG. 2). The dextramer pool consists of epitopes with known common viral and cancer reactivities across eight HLA alleles (table 2).

TABLE 1
Information on the T cell donors used in this study

| | HLA alleles | | | |
|---|---|---|---|---|
| Donor | HLA-A1 | HLA-A2 | HLA-B1 | HLA-B2 |
| Donor 1 | 02:01 | 11:01 | 35:01 | NA |
| Donor 2 | 02:01 | 01:01 | 08:01 | NA |
| Donor 3 | 24:02 | 29:02 | 35:02 | 44:03 |
| Donor 4 | 03:01 | 03:01 | 07:02 | 57:01 |
| Donor V | 02:01 | 29:02 | 35:01 | 57:01 |

TABLE 2
List of the dCODE Dextramer reagents used in the study.

| IMMUDEX | | | |
|---|---|---|---|
| Cat# | HLA allele | Peptide | Annotation |
| WC2197 | A*03:01 | KLGGALQAK | IE-1/CMV |
| WD2149 | A*11:01 | AVFDRKSDAK | EBNA 3B/EBV |
| WB2161 | A*02:01 | GILGFVFTL | Flu MP/Influenza |
| WI2148 | B*08:01 | RAKFKQLL | BZLF1/EBV |
| WD2175 | A*11:01 | IVTDFSVIK | EBNA 3B/EBV |
| WB2162 | A*02:01 | ELAGIGILTV | MART-1/Cancer/ |
| WB2130 | A*02:01 | GLCTLVAML | BMLF1/EBV |
| WC2656 | A*03:01 | RLRAEAQVK | EMNA 3A/EBV |
| WB2143 | A*02:01 | LLDFVRFMGV | EBNA 3B/EBV |
| WI2147 | B*08:01 | FLRGRAYGL | EBNA 3A/EBV |
| WB3529 | A*02:01 | FLYALALLL | LMP2A/EBV |
| WB2646 | A*02:01 | RTLNAWVKV | Gag protein/HIV |
| WB2141 | A*02:01 | LLFGYPVYV | HTLV-1 |
| WF2196 | A*24:02 | AYAQKIFKI | IE-1/CMV |
| WB3531 | A*02:01 | YLLEMLWRL | LMP1/EBV |
| WB5335 | A*02:01 | FLASKIGRLV | Ca2+-indepen.Plip A2 |
| WF2133 | A*24:02 | QYDPVAALF | pp65/CMV |
| WB2660 | A*02:01 | KTWGQYWQV | gp100/Cancer |
| WB3474 | A*02:01 | KVLEYVIKV | MAG E-A1/Cancer/ |
| WB2652 | A*02:01 | MLDLQPETT | 16E7/HPV |
| WH2165 | B*07:02 | QPRAPIRPI | EBNA 6/EBV |
| WB2158 | A*02:01 | IMDQVPFSV | gp 100/Cancer/ |
| WB3247 | A*02:01 | SLLMWITQV | NY-ESO-1/Cancer/ |
| WH2166 | B*07:02 | RPPIFIRRL | EBNA 3A/EBV |
| WB3340 | A*02:01 | SLFNTVATLY | Gag protein/HIV |
| WB2132 | A*02:01 | NLVPMVATV | pp65/CMV |
| WB3338 | A*02:01 | SLFNTVATL | Gag protein/HIV |
| WB2177 | A*02:01 | RMFPNAPYL | WT-1 |
| WB2191 | A*02:01 | YLNDHLEPWI | BCL-X/Cancer/ |
| WI2137 | B*08:01 | E LRRKMMYM | IE-1/CMV |
| WA2131 | A*01:01 | VTEHDTLLY | IE-1/CMV |
| WB3697 | A*02:01 | CLLWSFQTSA | Tyrosinase/Cancer/ |
| WB3497 | A*02:01 | KVAELVHFL | MAGE A3/Cancer/ |
| WB5066 | A*02:01 | CLLGTYTQDV | Kanamycin B dioxygenase |
| WB3307 | A*02:01 | LLMGTLGIVC | HPV 16E7, 82-91 |
| WB2144 | A*02:01 | CLGGLLTMV | LMP-2A/EBV |
| WB2139 | A*02:01 | ILKEPVHGV | RT/HIV |
| WF2639 | A*24:02 | CYTWNQMNL | WT1 (235-243)236M→Y |
| WB2157 | A*02:01 | KLQCVDLHV | PSA 146-154 |
| WB3339 | A*02:01 | SLYNTVATLY | Gag protein/HIV |
| WC2632 | A*03:01 | RIAAWMATY | BCL-2L1/Canced |
| WK2138 | B*35:01 | IPSINVHHY | pp65/CMV |
| WH2136 | B*07:02 | TPRVTGGGAM | pp65/CMV |
| WH2135 | B*07:02 | RPHERNGFTVL | pp65/CMV |
| NI3233 | NR(B*08:01) | AAKGRGAAL | NC |
| WB2666 | A*02:01 | ALIAPVHAV | NC |
| WF3231 | A*24:02 | AYSSAGASI | NC |
| WH3397 | B*07:02 | GPAESAAGL | NC |
| WA3580 | A*01:01 | SLEGGGLGY | NC |
| WA3579 | A*01:01 | STEGGGLAY | NC |

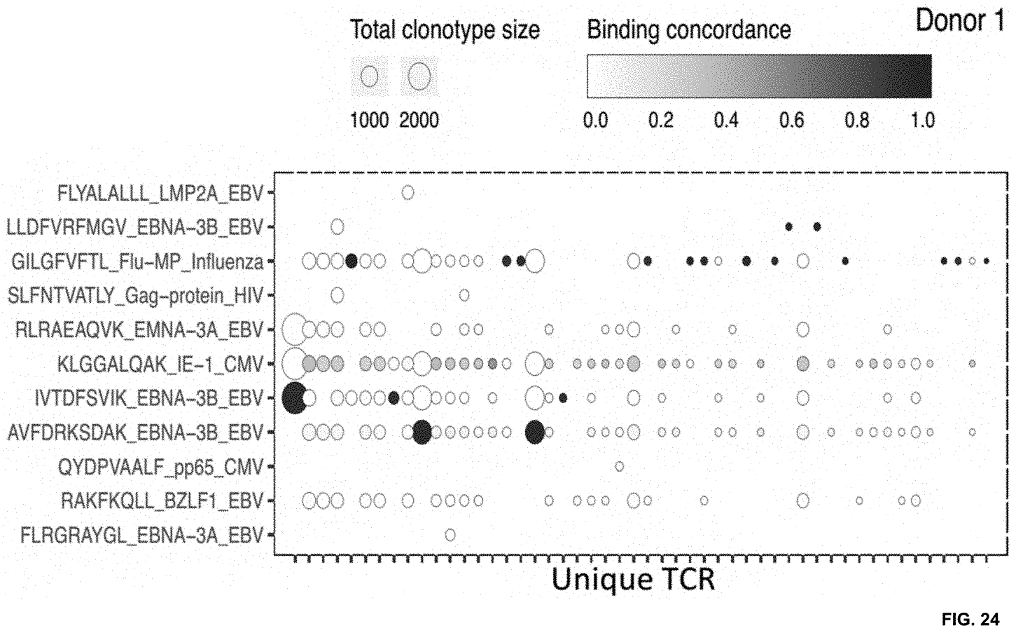

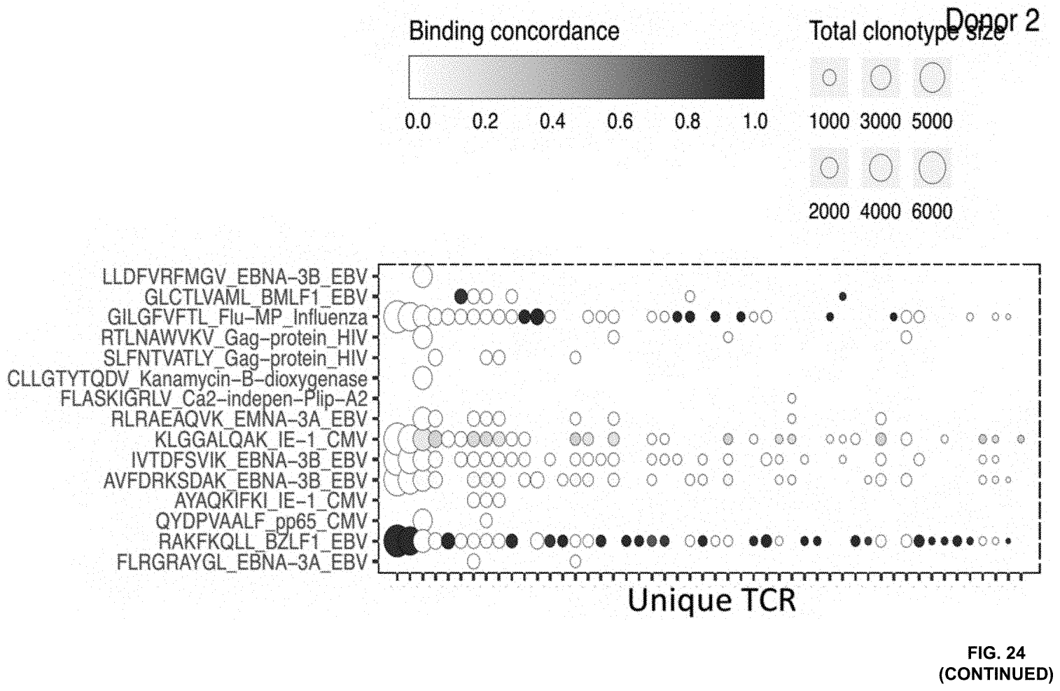

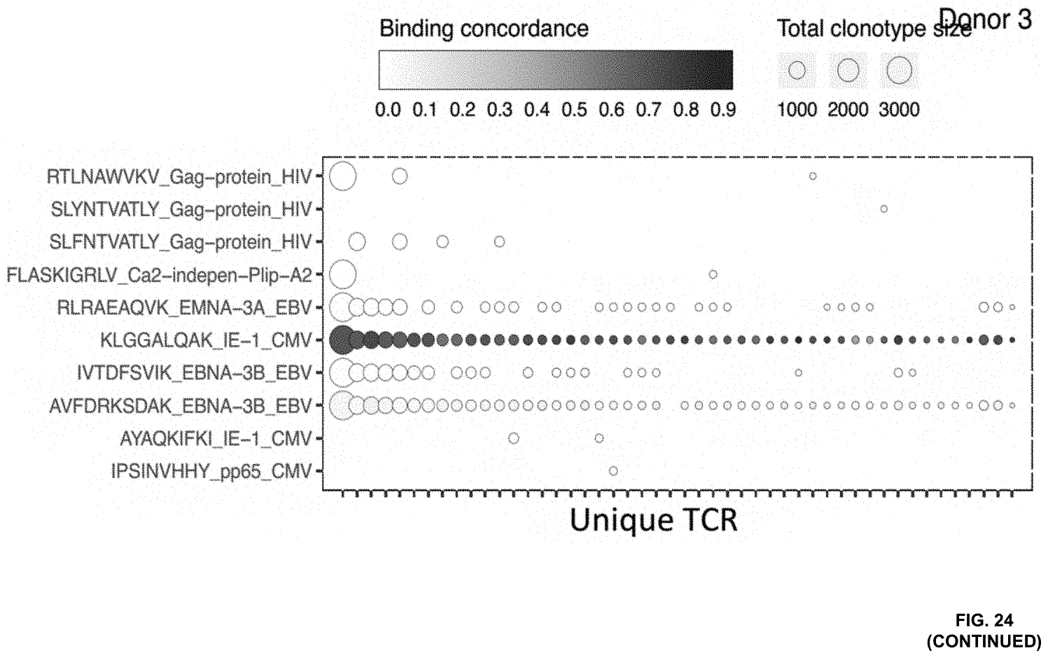

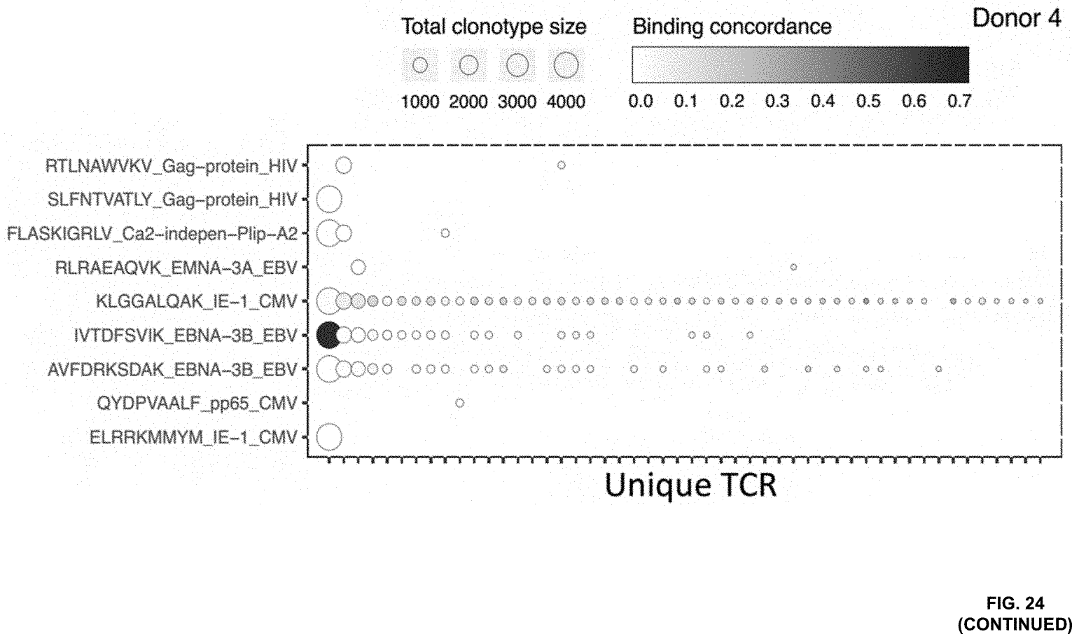

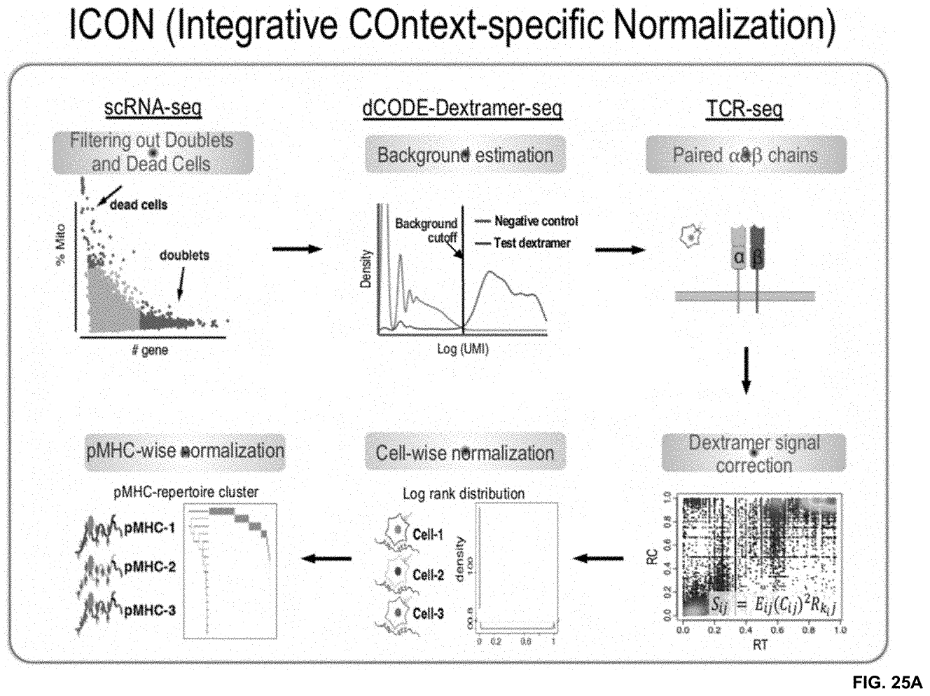

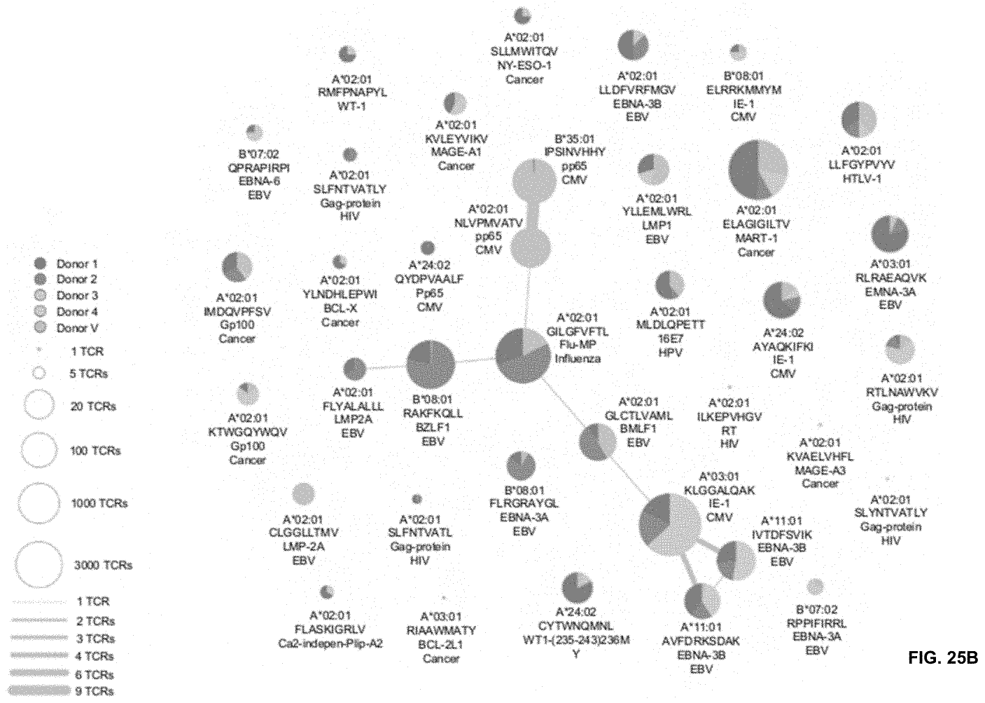

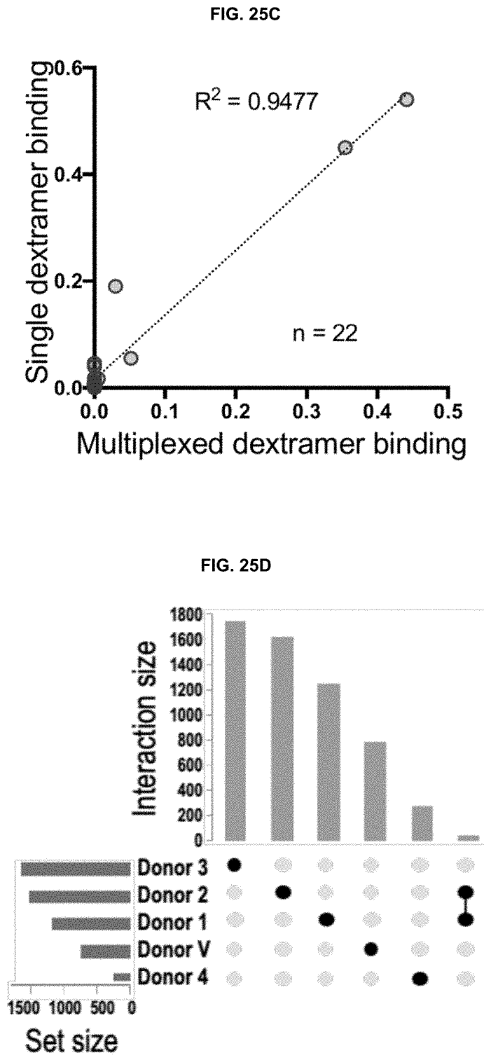

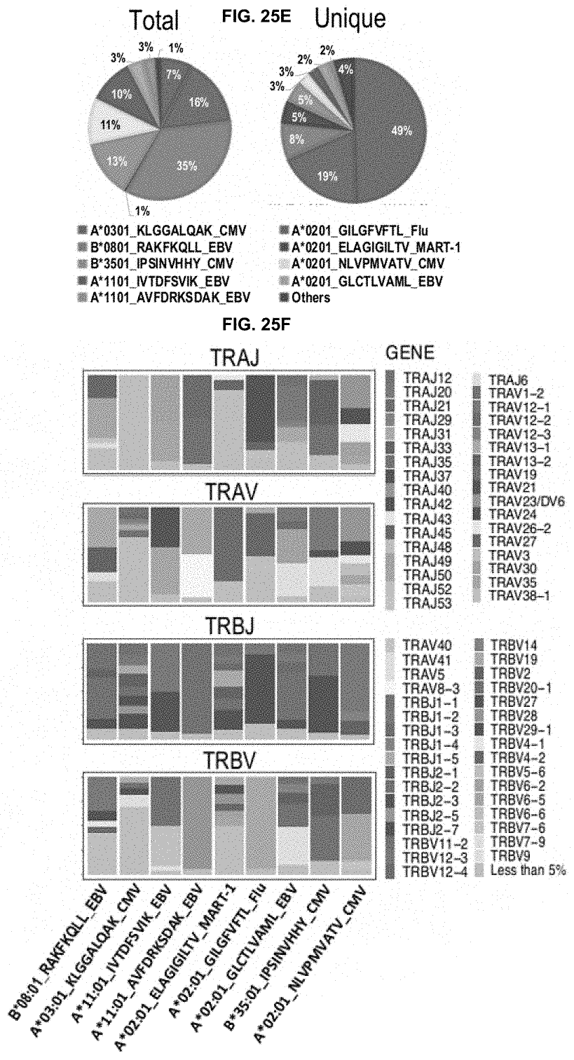

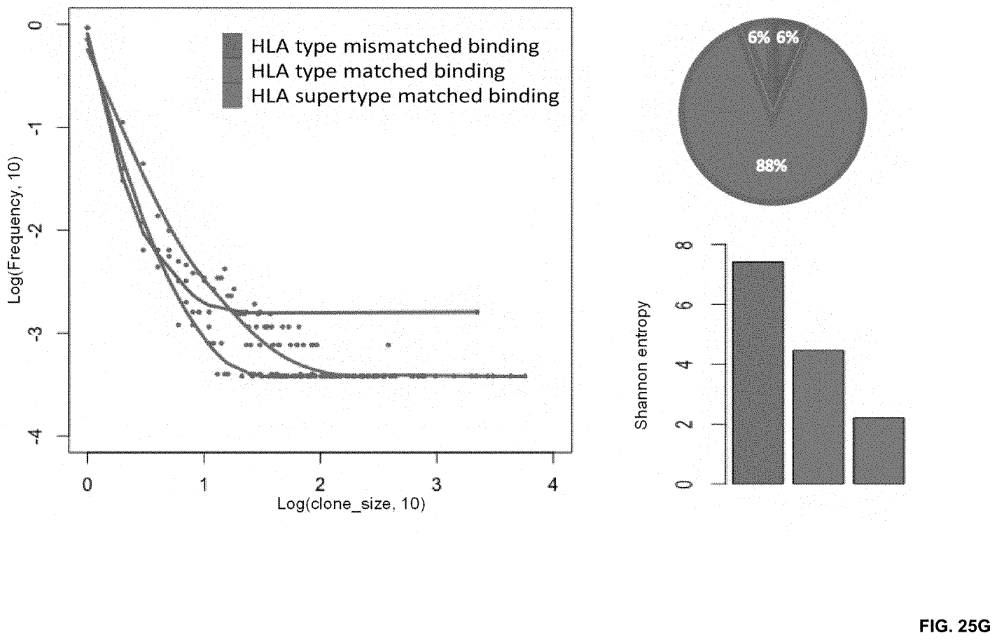

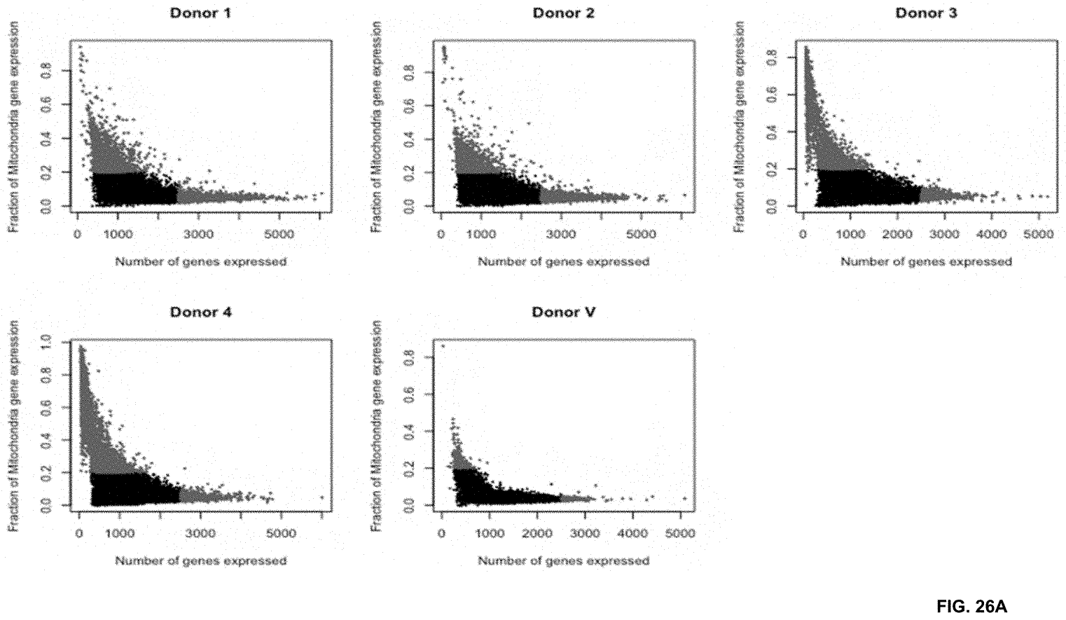

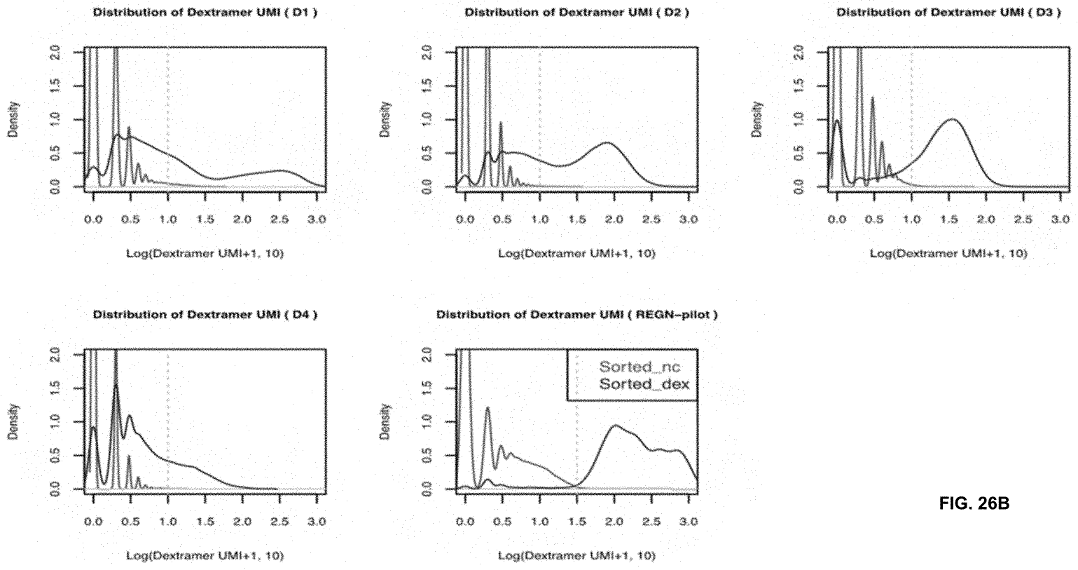

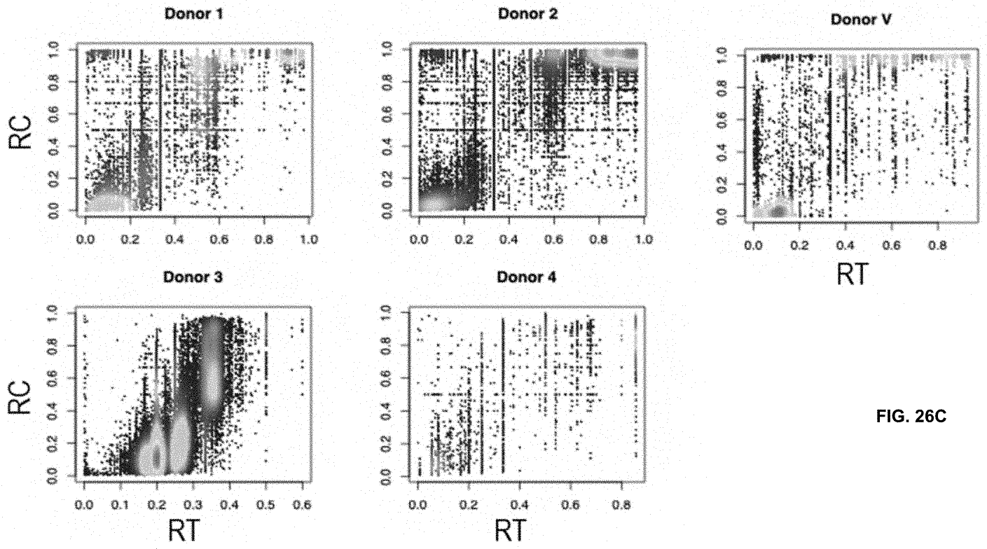

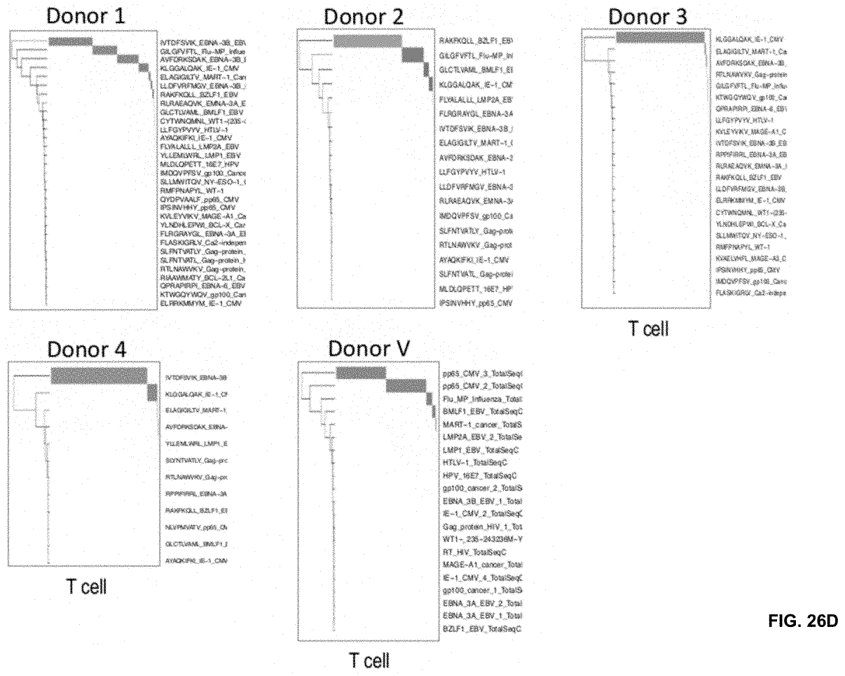

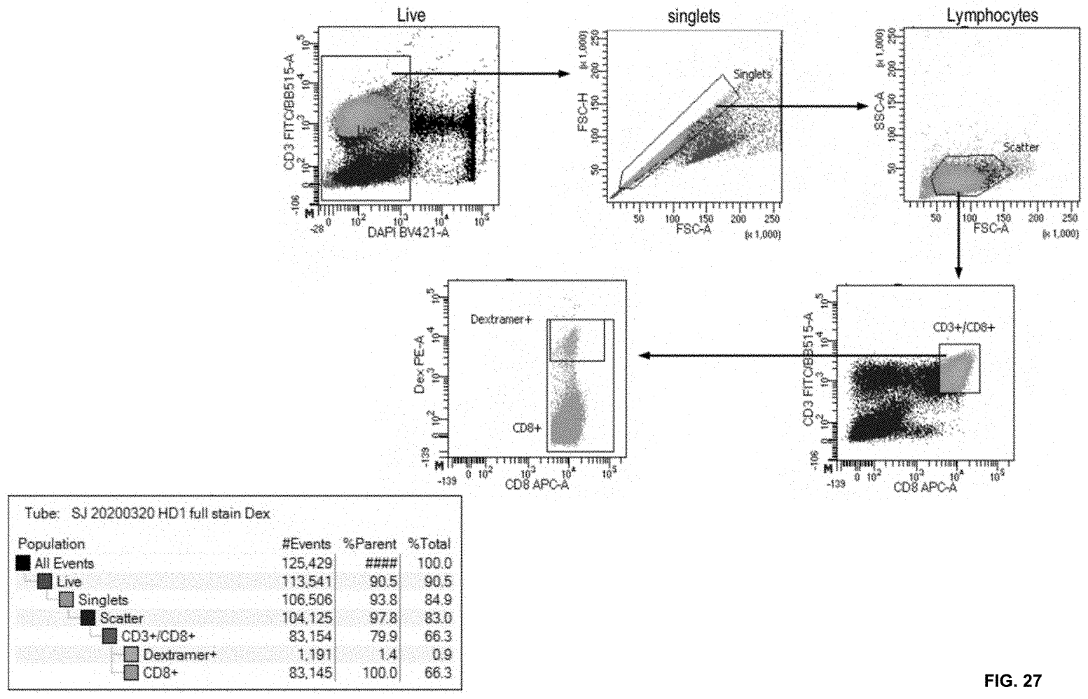

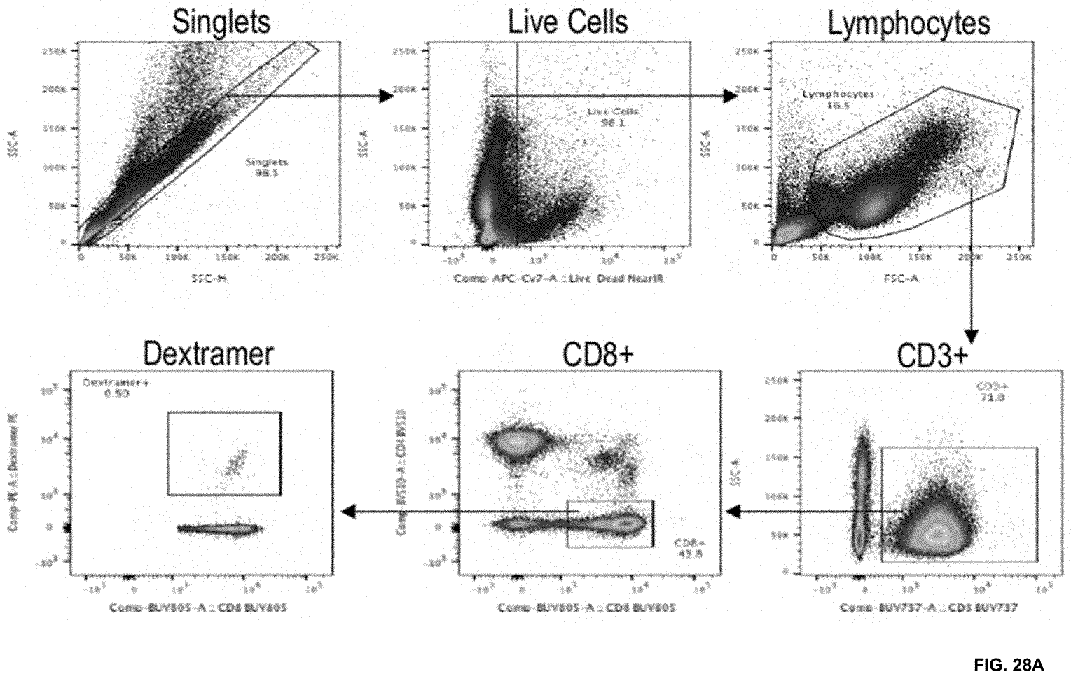

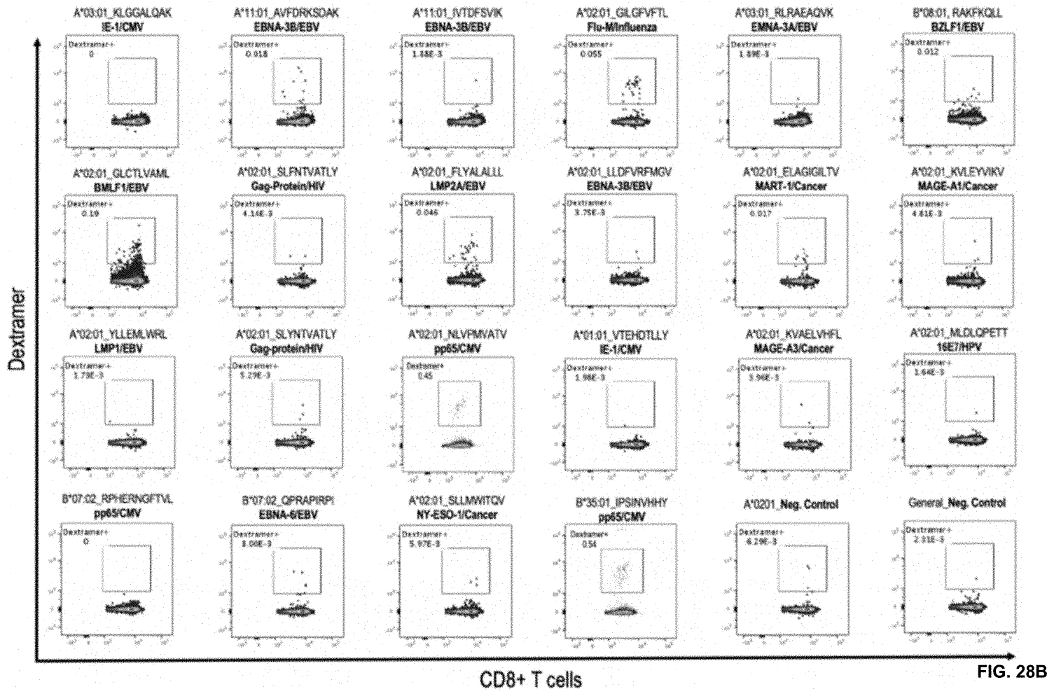

Described herein is a highly multiplexed dextramer binding dataset generated at the single cell level with paired T cell α- and β-chain sequences. 10× Genomics applied global cutoffs for background noise and non-specific dextramer bindings to all donors and dextramers to identify pMHC binding TCRs(18). Unsurprisingly, an unexpectedly high number of promiscuous TCR-pMHC binding events was found that 10× Genomics provided (FIG. 24). To robustly identify reliable binding events from such high-throughput TCR-pMHC binding data, ICON was developed (FIG. 25A, FIG. 26A-D and Materials and Methods). The ICON data process is performed in a donor, cell and dextramer specific context. In brief, single cell transcriptome data was used to select good quality cells (live and singleton). Then, negative control dextramers (n=6) were used to empirically estimate the background binding noise for each donor. Raw dextramer binding signals were subsequently corrected by subtracting the estimated background noise for each donor separately. T cells with paired αβ chains were selected as the candidates of pMHC binding T cells, as previous studies have demonstrated that pairing αβ synergistically drive TCR-pMHC recognition. T cell dextramer binding signals were further corrected by penalizing dextramers simultaneously binding to the same T cell/clone. Finally, dextramer binding signals were normalized across cells and pMHCs to make them directly comparable (FIG. 25A, FIG. 26A-D and Methods). To evaluate ICON performance, the pMHC binding specificities of CD8+ T cells were assessed from another healthy donor (donor V) using the same dextramer panel (FIG. 27 and Materials and Methods). ICON was able to link 91% of sequenced T cells with paired b αβ chains to their antigen targets. To estimate the specificity of ICON, 21 individual dextramer binding essays were conducted using the T cells from the same donor, donor V (ee and Materials and Methods). The flow cytometry result shows agreement with the relative abundance of T cells binding to these 21 dextramers identified from ICON (FIG. 25C).

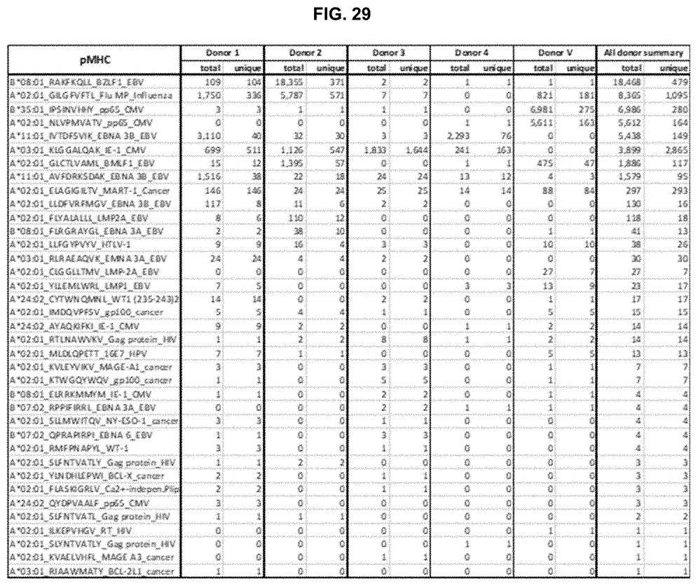

Applying ICON, a total of 53,062 CD8+ T cells belonging to 5,721 unique T cell clones that bind to 37 pMHCs from five donors were identified (FIG. 25B, FIG. 29). The dual specificity of TCRs (specificity versus degeneracy) has been suggested as an important feature of the immune response mechanism that sufficiently distinguishes self from foreign peptides to avoid autoimmune reactivity, while maintaining broad antigenic coverage. Indeed, 99.6% of unique TCRs bind to one specific pMHC and the remaining TCRs interact with 2 pMHCs (FIG. 25B). In addition, these TCR-pMHC interactions generally follow an HLA type specific pattern. 94% of binding events are HLA matched, of which 6% involve cross-recognition between HLA A*03-supertype family members HLA A*03:01 and A*11:01 that share similar main anchor positions of the presented peptide. Donors 1 and 2, who possess the most common HLA haplotype (A*02:01) in the dextramer pool (Table 1 & 2), share a significant fraction (n=44) of unique TCR-pMHC interactions (FIG. 25D, FIG. 25G), supporting the dogma that TCR-pMHC binding patterns are most likely to be HLA restricted. However, 6% of binding events are cross-HLA type interactions. HLA type mismatched binding T cells tend to have smaller clones or to be singletons (antigen naïve).

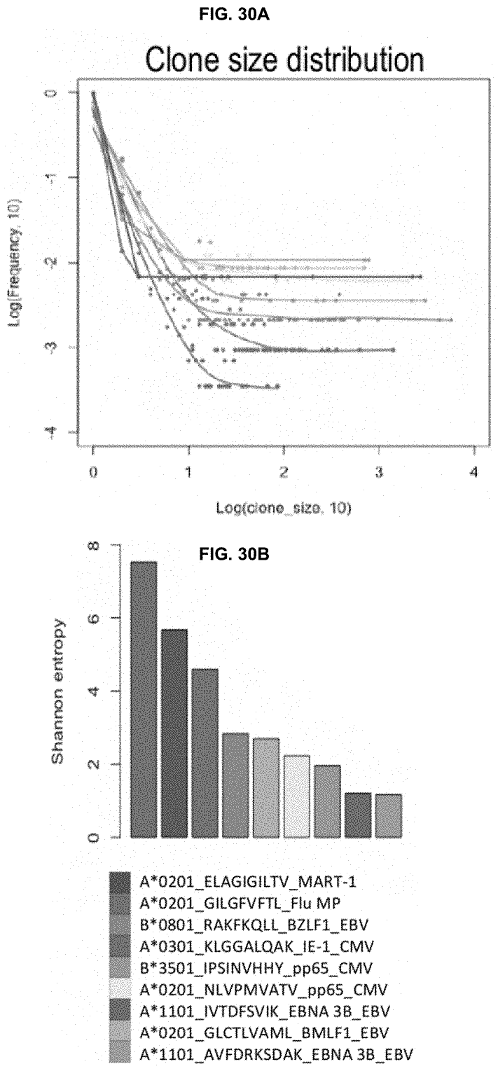

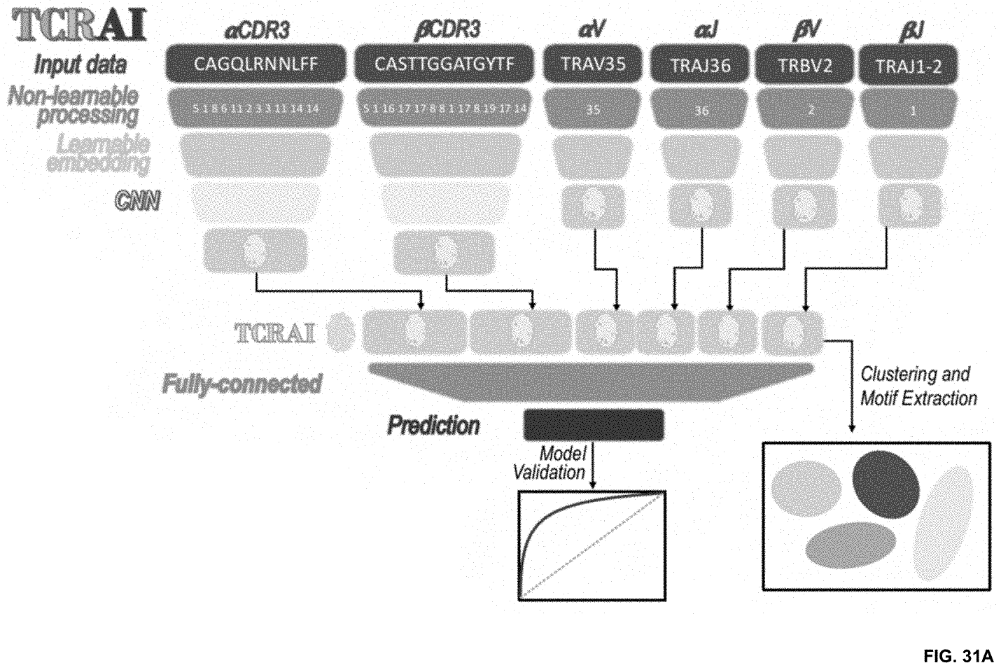

Among all pMHC binding TCRs, 99% of total TCRs (96% of unique TCRs) bind to nine pMHCs: B*08:01_RAKFKQLL_BZLF1_EBV (# of T cells: 18,468/# of unique TCRs: 479), A*02:01_GILGFVFTL_Flu-MP_Influenza (# of T cells: 8,365/# of unique TCRs: 1,095), A*11:01_IVTDFSVIK_EBNA-3B_EBV (# of T cells: 5,438/# of unique TCRs: 149), A*03:01_KLGGALQAK_IE-1_CMV (# of T cells: 3,899/# of unique TCRs: 2,865), A*11:01_AVFDRKSDAK_EBNA-3B_EBV (# of T cells: 1,579/# of unique TCRs: 95), A*02:01_GLCTLVAML_BMLF1_EBV (# of T cells: 1,886/# of unique TCRs: 117), A*02:01_ELAGIGILTV_MART-1_Cancer (# of T cells: 297/# of unique TCRs: 293), B*35:01_IPSINVHHY_pp65_CMV (# of T cells: 6,986/# of unique TCRs: 280) and A*02:01_NLVPMVATV_pp65_CMV (# of T cells: 5,612/# of unique TCRs: 164) (FIG. 25E). To further understand the conserved TCR sequence features underlying the classification, TCR VJ gene usages were examined for these nine pMHC repertoires. In addition to the enrichment that previous studies reported, such as TRBV19 and TRAV27 in the Influenza repertoire, TRAV5 and TRBV20-1 in the BMLF1_EBV repertoire, and TRBV6-5 in NLVPMVATV_pp65_CMV, abundant usage of TRAV12-2 in the MART-1 Cancer repertoire, TRAV21, TRAV35, TRBV11-2 and TRBV6-6 in the IVTDFSVIK_EBNA-3B_EBV repertoire, TRAV8-3, TRAV13-1 and TRBV28 in AVFDRKSDAK_EBNA-3B_EBV, TRAV13-1, TRAV13-2 and TRBV12-3 in the BZLF1_EBV repertoire, TRAV12-1, TRAV41, TRBV2 and TRBV20-1 in IPSINVHHY_pp65_CMV, and TRAV23/D6 and TRBV12-4 in NLVPMVATV_pp65_CMV were found (FIG. 25F). Consistent with the conserved VJ gene usage, Shannon diversity indexes and TCR clone size distributions suggested that each pMHC binding T cell repertoire experienced different degrees of expansion in responding to their target peptides (FIGS. 30A & B).

ii. TCRAI: A Neural Network Classifier of T Cell Antigen Specificity

With large and diverse TCR-pMHC binding events identified, robust functional classifiers for rapidly validating these binding events are desired. Recent work demonstrated that neural networks can learn high dimensional information from TCR sequences and thus, may robustly predict TCR-pMHC binding.

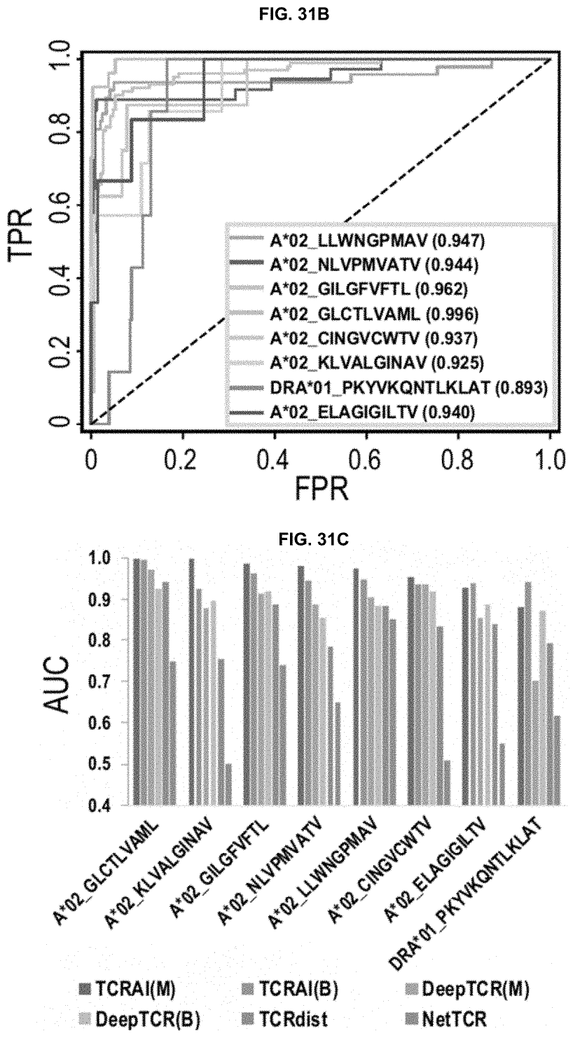

A Python package, TCRAI, has been developed utilizing Tensorflow 2, providing a flexible framework for the study of TCR-pMHC specificity (FIG. 31A). The highly modularized TCRAI package allows one to easily adjust the architecture of the model. In brief, the TCRAI framework works as follows. One can define any number of the V(D)J genes, and CDR regions of the TCR as inputs to the model in their textual form. One can then choose how to process these inputs into numerical form in a non-learnable way, via “processor” objects that convert text to numerical representations. These numerical inputs can then be further processed in learnable ways via “extractor” objects that form blocks of the neural network and give as their output vector representations of the input data, which are called fingerprints. These fingerprints are concatenated into a single TCRAI fingerprint describing this input TCR via a single numerical vector. This TCRAI fingerprint is then passed through a “closer” object which forms the final block of the neural network architecture, producing a prediction on the input TCR. The TCRAI package provides several such pre-built processors, extractors and closers, and is easily extendible to new variants. It also allows one to perform binomial, multinomial, regression or other tasks by simply choosing to construct a different closer object.

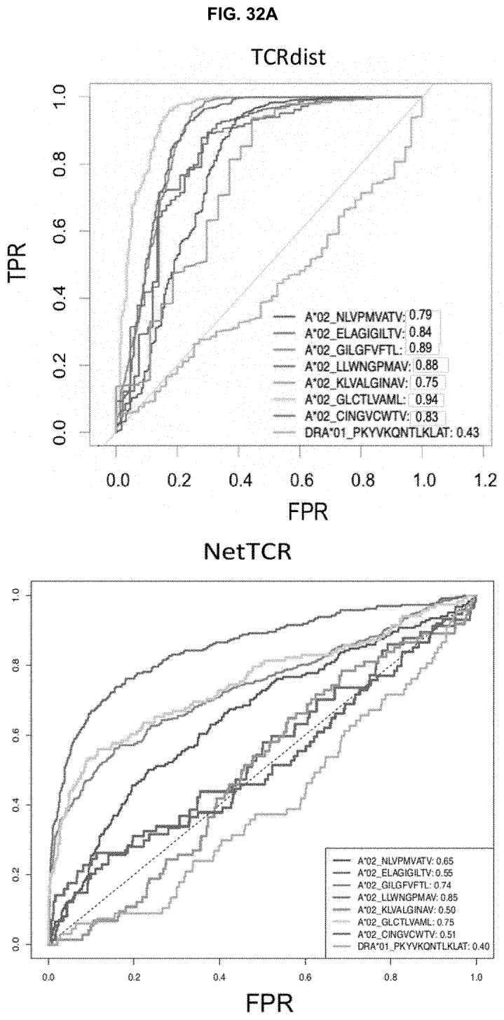

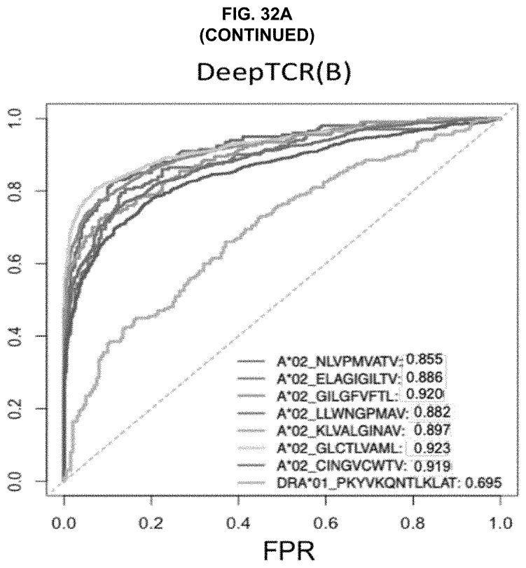

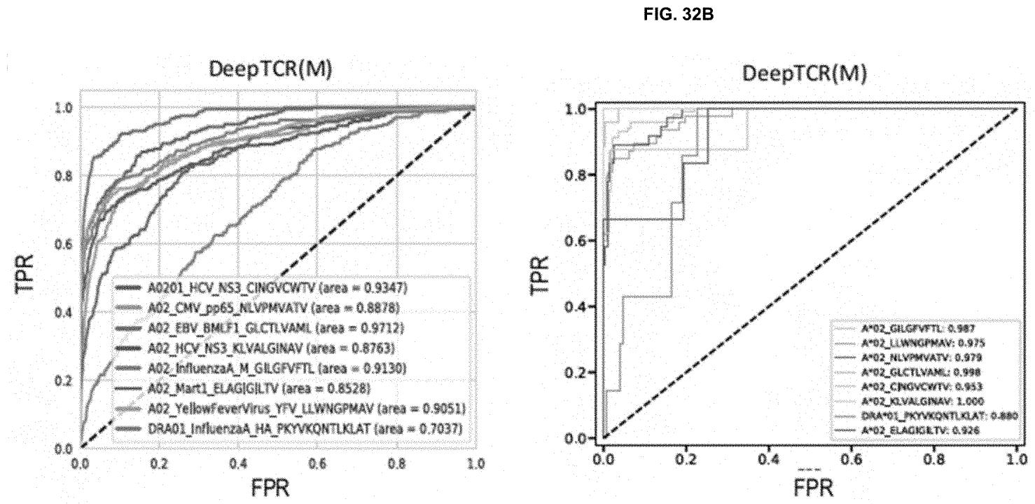

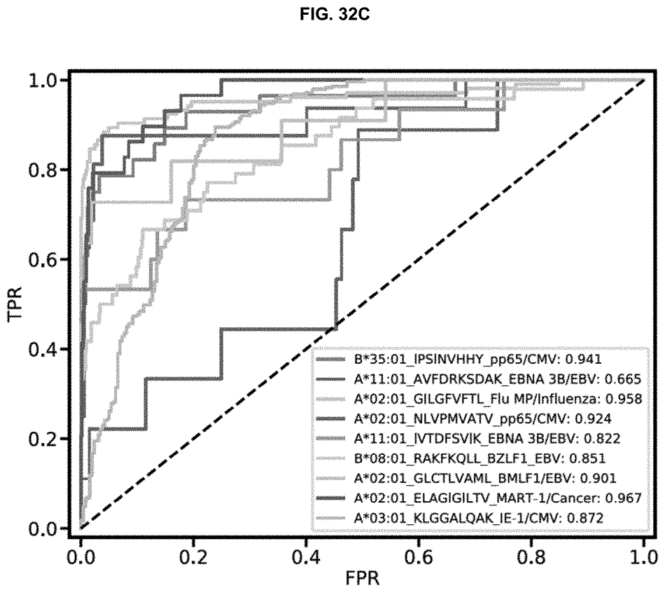

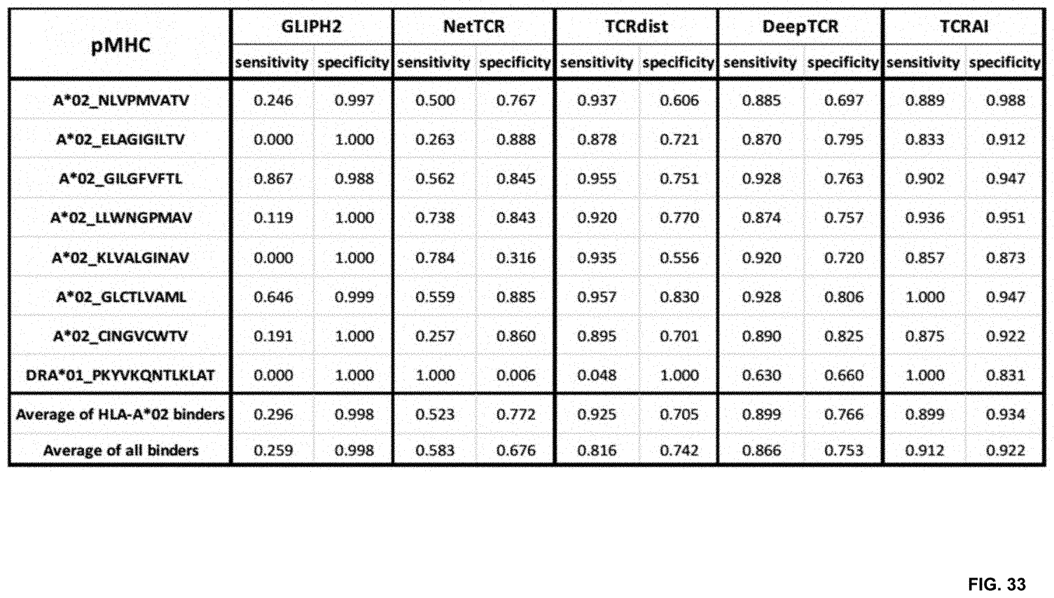

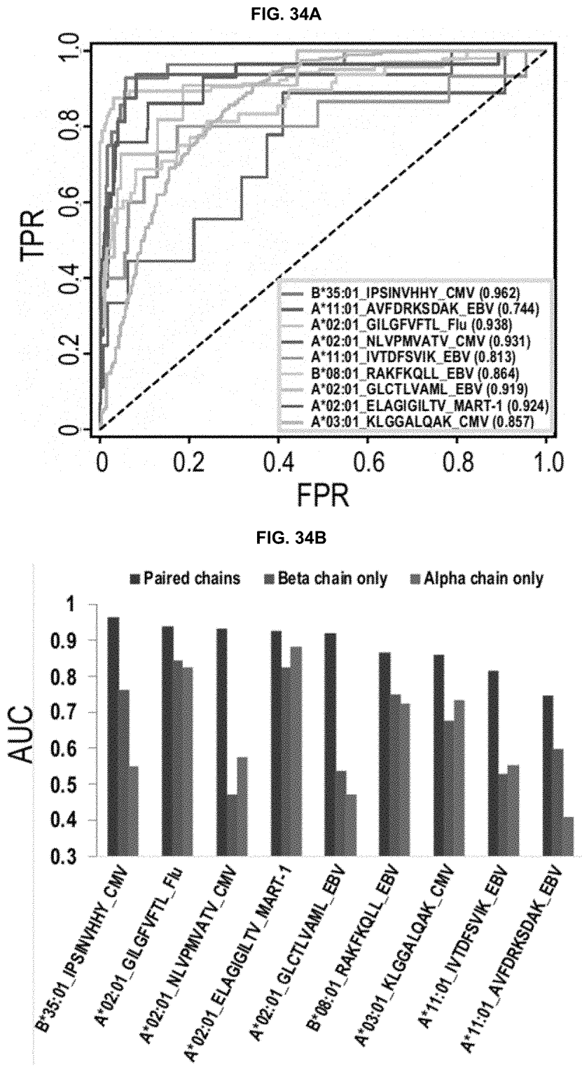

To evaluate the performance of TCRAI, a literature search for currently available methods was performed (table 3) and the classifier was compared to four major methods in this field: GLIPH2, DeepTCR, NetTCR and TCRdist. For the comparison, eight pMHC-specific binding T cell repertoires were collated with at least 50 unique paired αβ chain TCRs generated by traditional single multimer binding or antigen re-exposure assays as a gold-standard dataset (table 4 and Materials and Methods). Three of the methods DeepTCR, NetTCR and TCRdist are, like TCRAI, predictive models. The area under the ROC (receiver operator characteristic) curve (AUROC/AUC), a standard measure of classification success, of these prediction models indicates that TCRAI and DeepTCR, with similar neural network frameworks, perform better than TCRdist and NetTCR. Overall, TCRAI has more consistent and better performance than DeepTCR (FIG. 31e, FIG. 32B, and FIG. 32C). Since GLIPH2 was designed for clustering TCR sequences into distinct groups of shared specificity, sensitivity and specificity (calculated at the model threshold which maximized the geometric mean of the two) of these four prediction models were measured in order to compare with GLIPH2. The comparison result demonstrated that TCRAI has the best-balanced sensitivity and specificity (FIG. 33). A couple of methods with different purposes to that of TCRAI were not included in the comparison. For example, ALICE is for detecting groups of homologous/expanded TCRs. TcellMatch uses cell-specific covariates (e.g. gene expression) but not TCR sequence alone as input and its performance was tested on the high noise to signal ratio 10× Genomics Immune Map data without further cleanup.

SUPPLEMENTARY TABLE 3
Summary of methods for linking TCR-antigen specificities

| Method | Function | Approach | Reference |
|---|---|---|---|
| GLIPH2 | Clustering TCRs that are predicted to | K-mer enrichment-based detection | Glanville et al. Nature, 2017 |
| | bind the same pMHC | of TCR motifs | |
| NetTCR | Predicting TCR-antigen specificty | Convolutional neural networks | Jurta et al. bioRxiv, 2018 |
| | | TCR sequence based | |
| | | HLA__A*02 restricted | |
| TCRdist3 | Predicting TCR-antigen specificty | Sequence similarity distance | Dash et al. Nature, 2017 |
| DeepTCR | Predicting TCR-antigen specificty | Convolutional neural networks | Sidhom et al. bioRxiv, 2019 |
| | | TCR sequence based | |
| ALICE | Detecting groups of homologous TCRs | Val rearrangement model | Pogorelyy et al. Genome Med, 2018 |
| TcellMatch | Predicting TCR-antigen specificities | Deep learning architecturesTCR | Fischer et al. Mol Syst Biol. 2020 |
| | | sequences + cell-specific covariates | |
| *TCRex | Predicting TCR-antigen specificty | Random forest classifier | Gielis et al. bioRxiv, 2018 |
| | | TCR sequence based | |

*TCRex: a webtool, available for academic, non-personal research only

TABLE 4
Summary of eight pMHC repertoires collated from VDJdb
and McPAS (Methods)

| | | Number of |
|---|---|---|
| pMHC | Peptide | unique TCR |
| A•02_InfluenzaA_M_GILGFVFTL | GILGFVFTL | 1187 |
| A•02_YellowFeverVirus_YFV_LLWNGPMAV | LLWNGPMAV | 525 |
| A•02_CMV_pp65_NLVPMVATV | NLVPMVATV | 390 |
| A•02_EBV_BMLF1_GLCTLVAML | GLCTLVAML | 274 |
| A•02:01_HCV_NS3_CINGVCWTV | CINGVCWTV | 83 |
| A•02_HCV_NS3_KLVALGINAV | KLVALGINAV | 75 |
| DRA•01_InfluenzaA_HA_PKYVKQNTLKLAT | PKYVKQNTLKLAT | 70 |
| A•02_MART-1_Cancer_ELAGIGILTV | ELAGIGILTV | 57 |

iii. Classification of pMHC Binding TCRs Identified from the High-Throughput Data

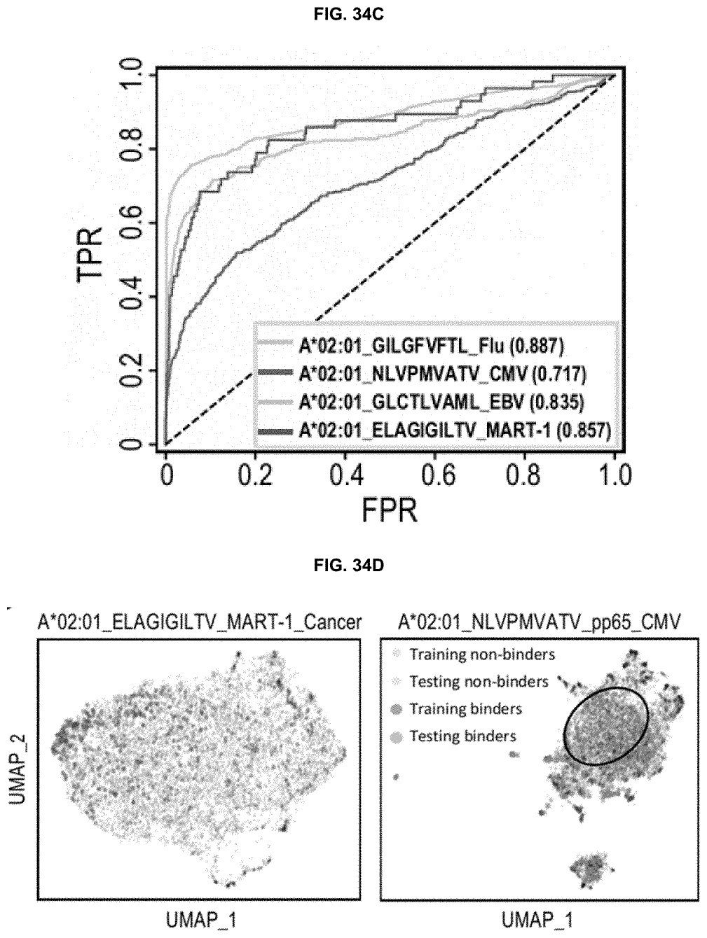

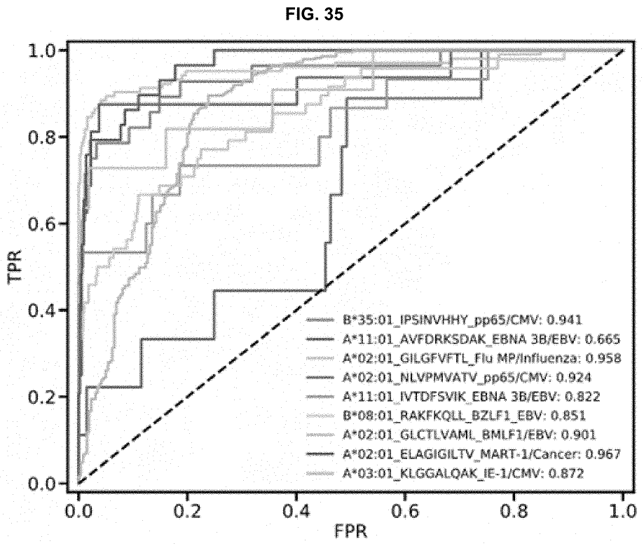

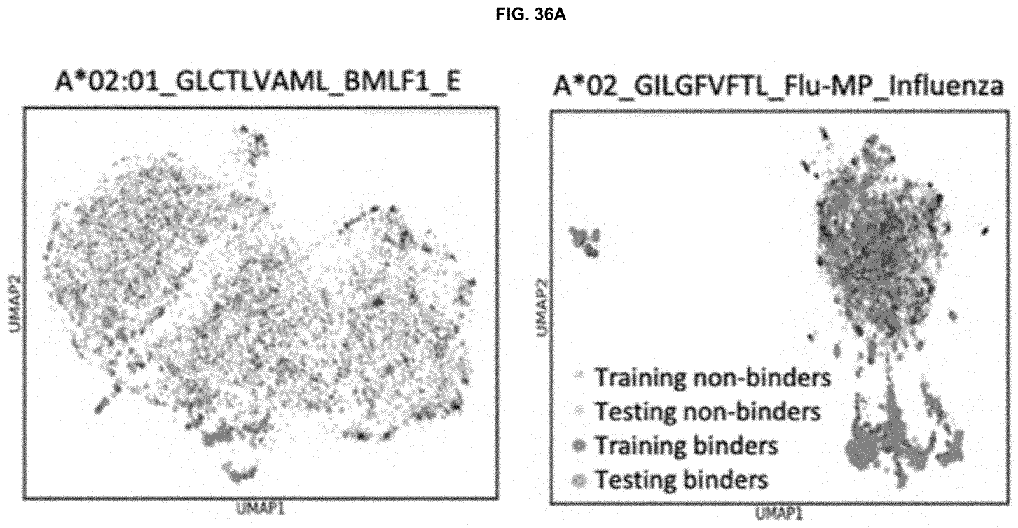

TCRAI was then applied to the nine most abundant pMHC binding repertoires ICON identified from the high throughput data (FIG. 25E). TCRs of these nine pMHC repertoires were classified with an average AUC 0.88 with TCRAI in binomial mode. Similar prediction performance was also seen using TCRAI multinomial mode (FIG. 34A and FIG. 35, hereinafter, TCRAI results are from the binary mode unless specified). Historically, TCR β-chain sequencing was often used to infer T cell antigen binding specificity due to its higher combinatorial potential compared to the α-chain. To quantitatively evaluate the contribution of TCR α- and β-chains in predicting TCR-pMHC interaction, either the α-chain or β-chain was used in lieu of paired αβ chains as input to TCRAI. The performance with paired αβ chains is better than α- or β-chain alone with an average increase of about 0.2 in the AUC (FIG. 34B). Consistent with previous studies, these results collectively demonstrate the importance of αβ pairing for accurate inference of TCR-pMHC interactions. The predictive performance for β-chains is not always better than a-chains, indicating the importance of α-chains in TCR-pMHC specific recognition, which was often overlooked previously.

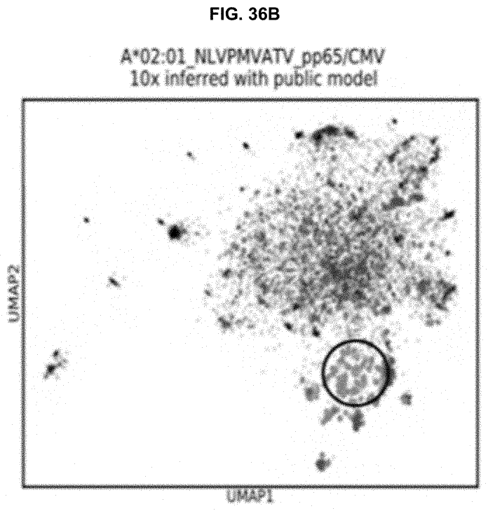

To further validate the performance of TCRAI, four pMHC repertoires (A*02:01_ELAGIGILTV_MART-1, A*02:01_GILGFVFTL_Flu-MP, A*02:01_GLCTLVAML_BMLF1_EBV and A*02:01_NLVPMVATV_pp65_CMV) were used that also have binding TCRs in the curated public dataset. TCRAI was trained using the four repertoires identified from the high throughput dataset to predict the four curated repertoires. FIG. 34C shows that prediction results are generally comparable to the performance on the training set. However, the performance of TCRAI when inferring on A*02:01_NLVPMVATV_pp65_CMV was significantly worse than the other three pMHCs. To understand the performance difference, the TCRAI fingerprint space of the model was investigated (Materials and Methods). In the case of A*02:01_ELAGIGILTV_MART-1_Cancer, and the other two pMHCs (FIG. 36A), binding TCRs from the high throughput dataset and the curated dataset overlap spatially in fingerprint space, whereas the overlap is significantly worse for the case of pp65_CMV (FIG. 34D and FIG. 36B). This poor overlap is attributed to 98.2% of pp65_CMV binding TCRs in the high throughput dataset coming from a single donor (FIG. 29), thereby representing a small subspace of possible binding TCRs, whereas the public data contains TCRs from a range of donors representing a larger range of the TCR space. This result also highlights the importance of large diverse datasets for training a robust TCR-antigen prediction model.

iv. Characterization of pMHC Specific TCRs

To investigate the properties of TCRs that bind a given pMHC, how TCRAI classifier models arrange TCRs within their fingerprint space were analyzed (Materials and Methods). TCR fingerprints from a classifier model allow for the discovery of specific groups of TCRs with conserved gene usage and CDR3 motifs. These groups often exhibit different binding abilities and divergent structural binding modalities.

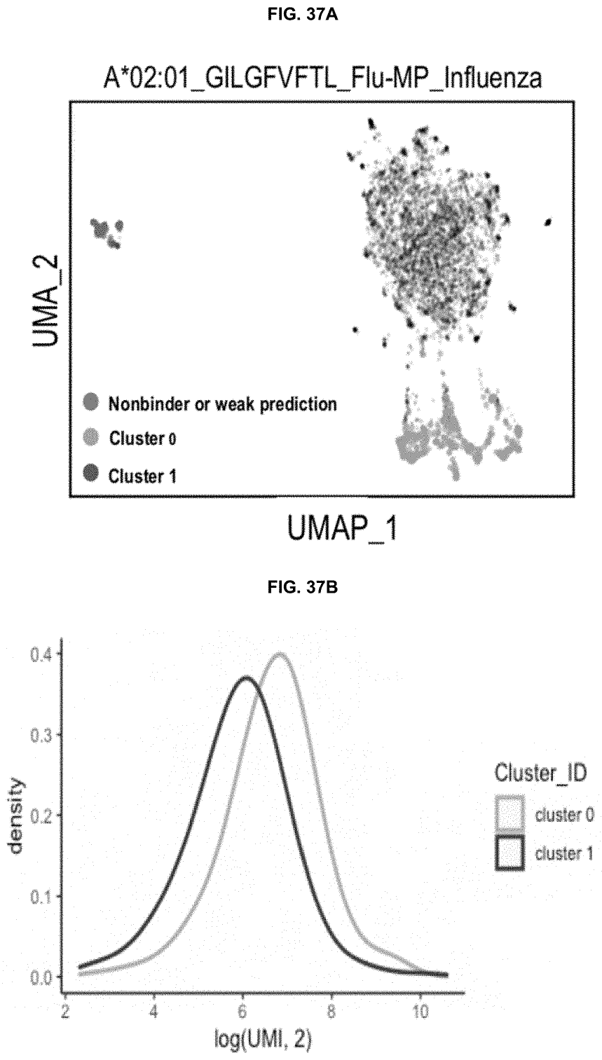

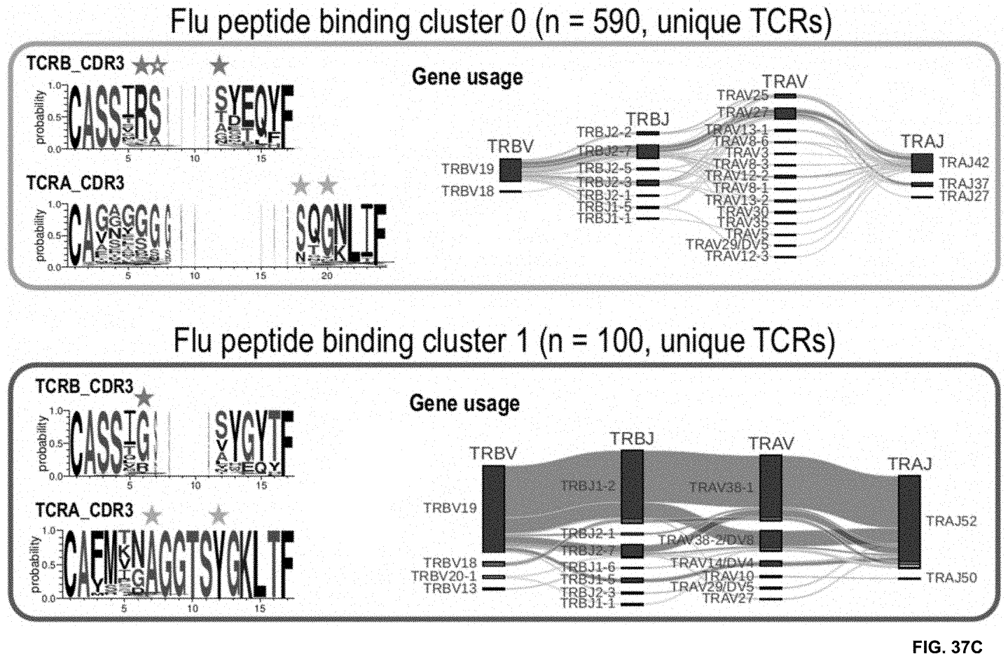

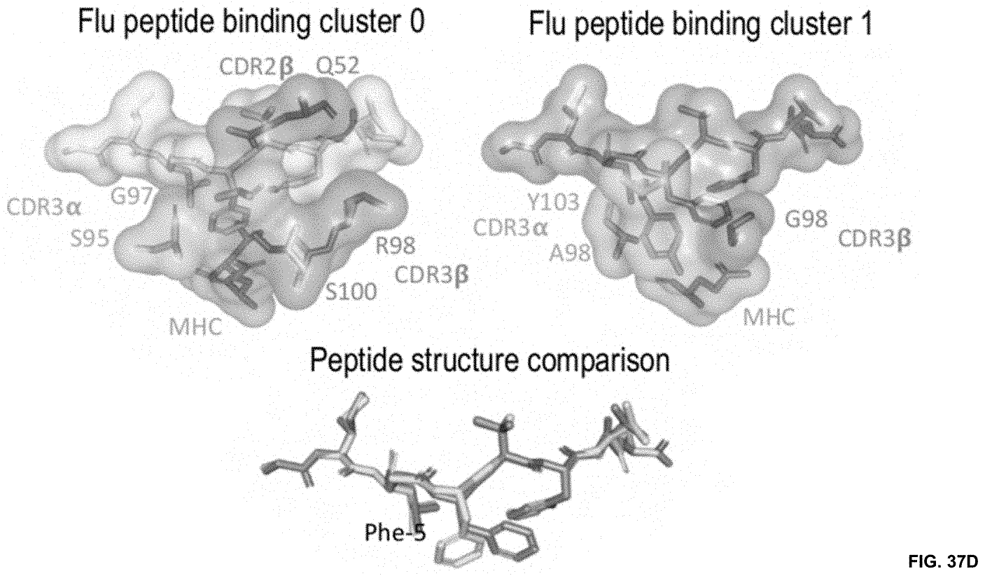

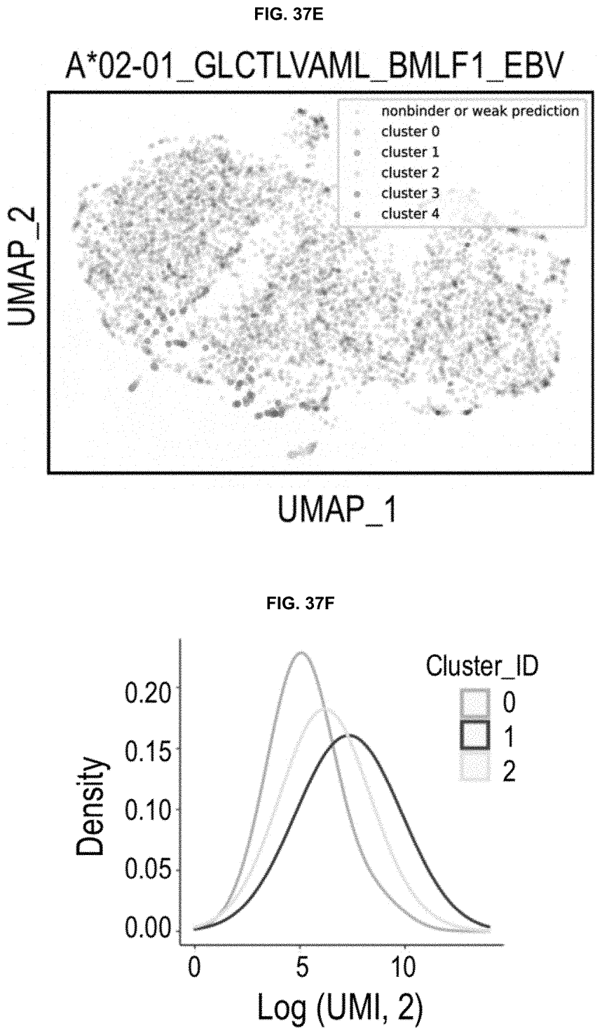

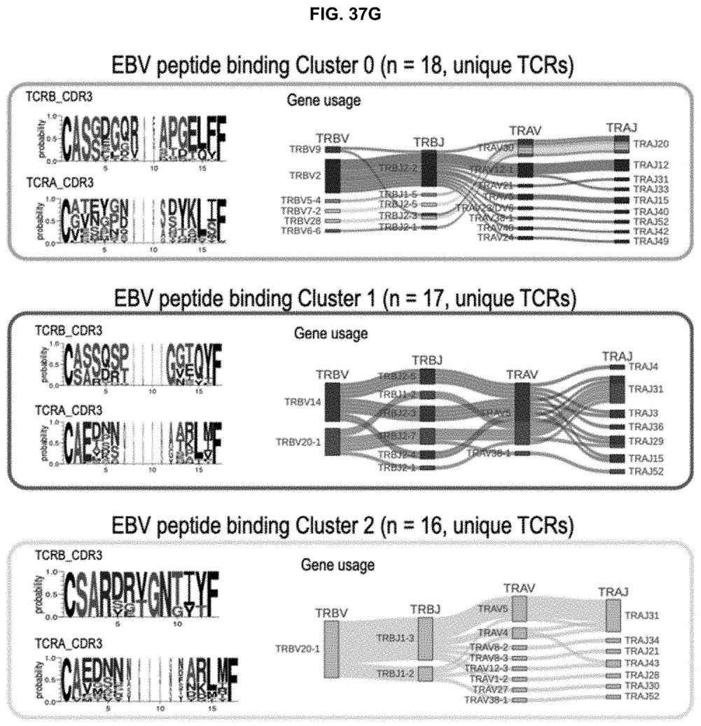

Clustering TCRs to A*02:01_GILGFVFTL_Flu-MP_Influenza leads to two well-separated clusters in the TCRAI fingerprint space (FIG. 37A). The constructed α- and β-CDR3 motifs and the gene usage indicate that the cluster 0 has a strongly conserved xRSx motif and TRB19 and TRAJ42 gene usage in the β-chain, and the smaller group cluster 1 has very highly conserved gene usage TRBV19/TRBJ1-2/TRAV38-1/TRAJ52 (FIG. 37C). The dextramer signal (in UMI, Unique Molecular Identifier) distribution indicated that TCRs in cluster 0 have stronger binding to the Flu dextramer than those in cluster 1 (FIG. 37B). The result is consistent with the well-known strong conservation of CDR3 motifs and TCRBV19 gene usage in A*02:01_GILGFVFTL_Flu responsive T cells thought to be connected to its “featureless” pMHC complex. Further comparing to the classes of A*02:01_GILGFVFTL_Flu binding TCRs recently identified, clusters 0 and 1 were linked to their Groups I (canonical) and II (novel), respectively. It was also found in the art the Group I TCRs have stronger binding than those in Group II. The 3D structures of the TCR-pMHC binding complexes proposed in the art suggest that due to highly conserved motifs/residues, these two groups of TCRs have different binding modalities, which cause the different Phe-5 ring rotation of the Flu peptide in these two complexes (FIG. 37D).

The TCRs binding to the other eight pMHCs were also characterized. The result for A*02:01_GLCTLVAML_BMLF1_EBV binding TCRs is particularly interesting. In previous studies, a dominant public TCR constructed from TRBV20-1/TRBJ1-2/TRAV5/TRAJ31 has been observed. However, previous analyses of the TCR population binding to this pMHC have focused on TRAV5 TCRs, to which the population is heavily biased. The current experiments unbiasedly identified 5 clusters of TCRs in the TCRAI fingerprint space (FIG. 37E). Clusters 1 and 2 represent the classic HLA*02:01_GLCTLVAML public TCRs, albeit split into two clusters based on their β-chain gene usage (FIG. 37G). Cluster 0 contains TCRs following a gene usage (TRBV2/TRBJ2-2) and β-chain CDR3 motif that have not presented elsewhere. TCRs belonging to this novel group show a different binding ability to the canonical TCR clusters (clusters 1 and 2), as can be seen from the reduced dextramer UMI count (FIG. 37F), which indicates a lower affinity and would partially explain why this group of TCRs has not yet been noted.

v. Immune Phenotypes of pMHC Binding CD8+ T Cells

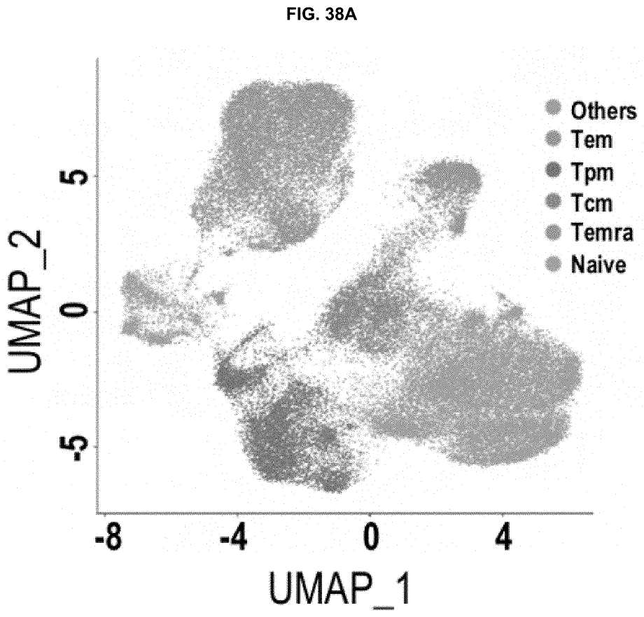

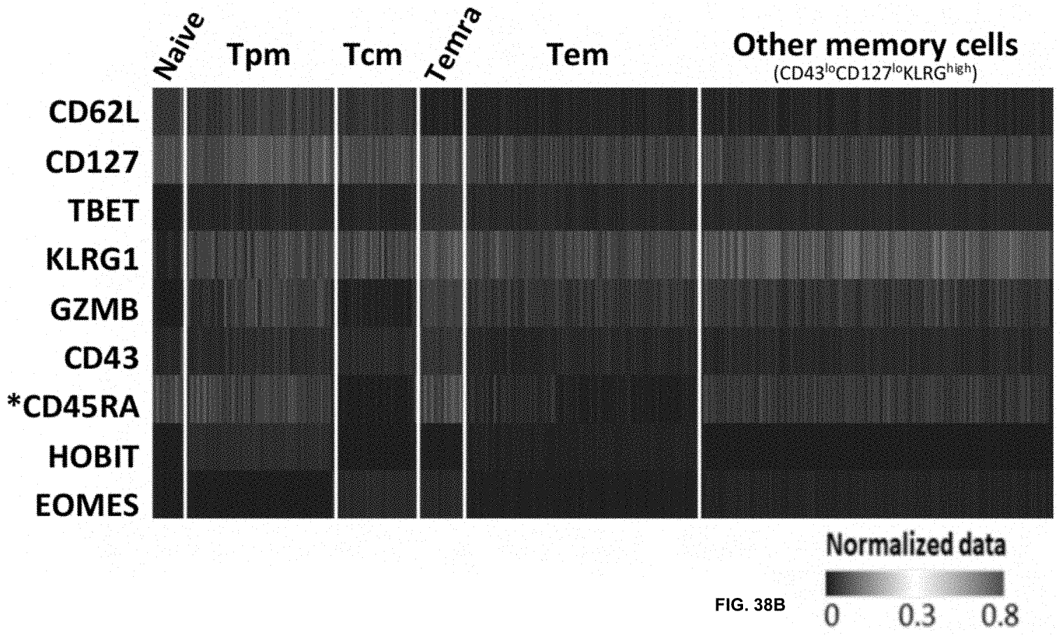

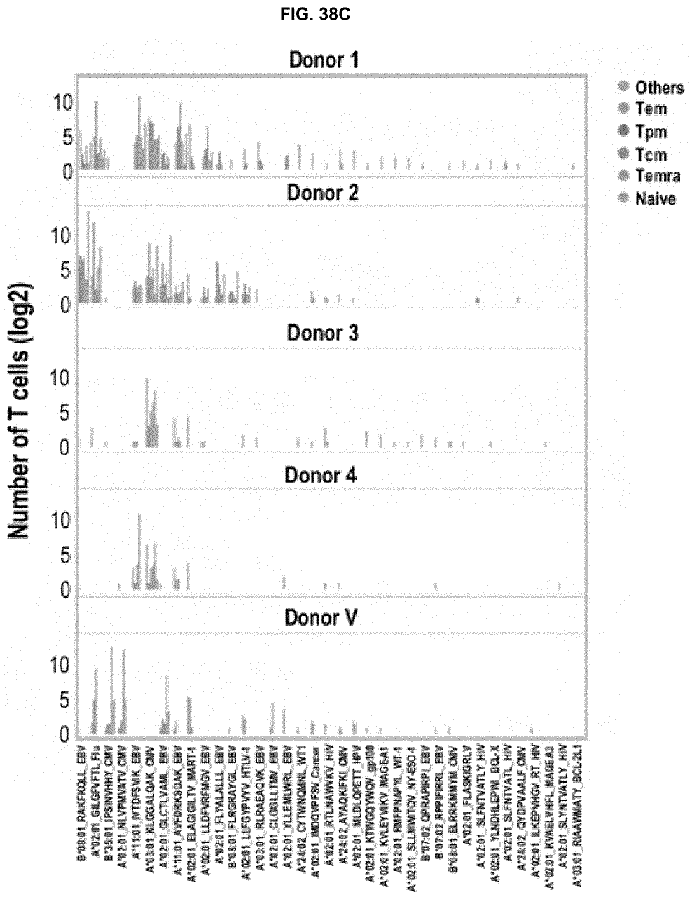

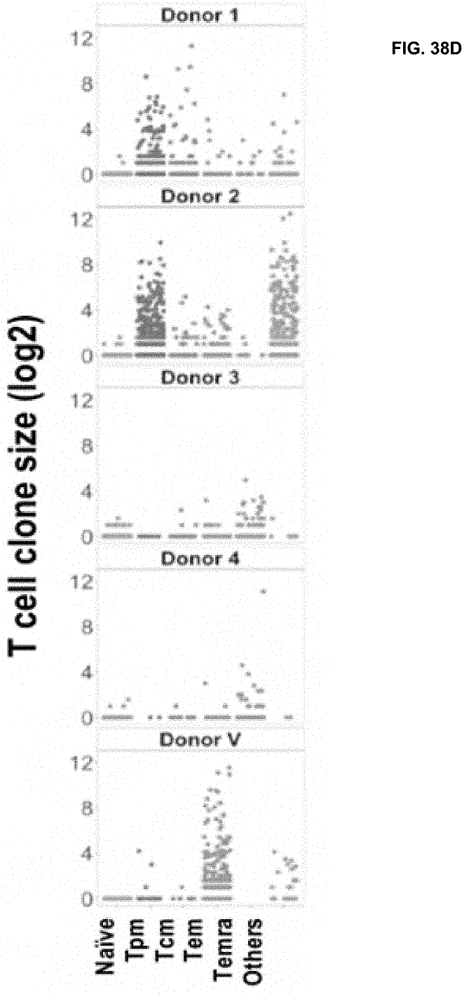

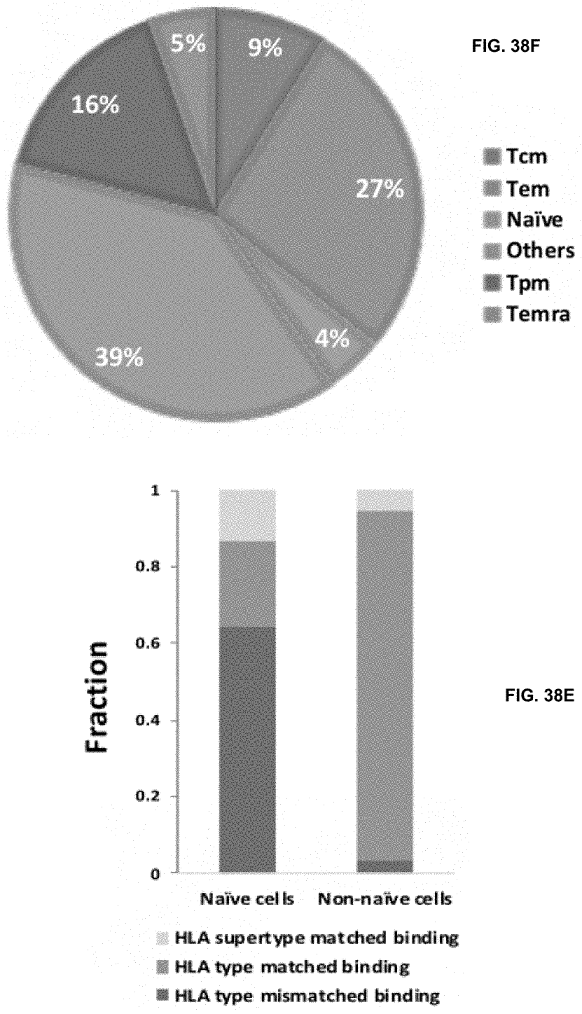

The combined information of antigen specificity and T cell phenotype has been reported to be important to clinical success of immunotherapies, such as vaccination. The multi-omics data generated by the Immune Map platform enables the association of T cell antigen specificity with T cell phenotypes. Using gene (single cell RNA-seq) and surface protein (CITE-seq, cellular indexing of transcriptomes and epitopes by sequencing) expression from this multi-omics dataset, pMHC binding CD8+ T cells was grouped into subpopulations (FIG. 38A and Materials and Methods). The identified subpopulations were then annotated according to CD8+ T cell subtype marker genes described previously: naïve cells (CD45RA+CD62LhiCD127hi), central memory cells (Tcm, CD45RA-CD62L+CD127+EOMEShighTBETlow), T effector memory cells (Tem, CD45RA-CD62LlowCD127+GZMB+), peripheral memory cells (Tpm, CD62L+CD127hiGZMB+), terminally differentiated effector cells (Temra, CD45RA+CD127loGZMBhi) and other memory cells (CD43loKLRG1hiCD127−) (FIGS. 38A & B).

96% of pMHC binding T cells were memory cells and were enriched in expanded T cell clones (FIGS. 38E & D), indicating that these T cells were selected by specific immune responses and thus are likely to be responsive and reliable binders. The majority of these memory T cells bound to common viral epitopes (e.g., influenza, EBV, CMV), and pMHC binding T cells from each donor demonstrated different distributions of memory cell subsets. For example, donors 1 and 2 had primarily Tpm, whereas donor V had Tem, and donors 3 and 4 had mostly Temra cells (FIGS. 38C & D).

Although the majority of pMHC binding T cells expressed a memory phenotype, 4% of them were naïve cells. These naïve cells had more diverse pMHC interactions than non-naïve cells and were often bound to tumor-associated antigens (e.g. MART-1), endogenous antigens, or to antigens derived from viruses for which the donor was purportedly seronegative (e.g. HPV) (FIG. 38C). Interestingly, the proportion of naïve T cells with cross-HLA type binding was significantly higher than of non-naïve cells (FIG. 38F). These results indicate that healthy donor T cell repertoires—particularly naïve cells—have the potential to respond to not-yet encountered or rare antigens and to retain cross-reactivity. Additional assays are required to assess whether these cells could mount a functional T cell response.

2. Discussion

High-throughput TCR-pMHC binding data present an attractive pathway for furthering the understanding of TCR antigen recognition. However, this type of data is often associated with high noise to signal ratios. Herein is presented a framework of computational tools including a novel method ICON that can identify reliable TCR-pMHC interactions by significantly increasing signal-to-noise ratios in the highly multiplexed TCR-pMHC binding data with good sensitivity and specificity. ICON computes the noise corrected dextramer signal in a parameter free manner, making it easily generalizable to pMHC-TCR binding data from a broader range of pMHC dextramer pools and potentially extendible to the normalization of protein binding signals in single cell space, such as CITE-seq.

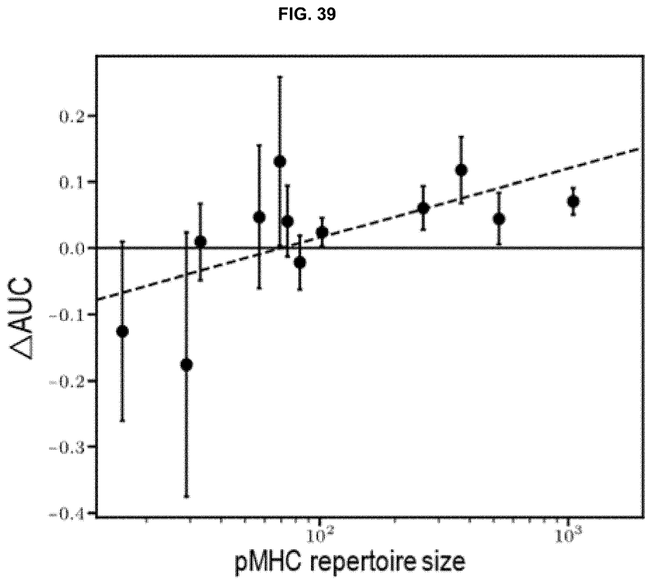

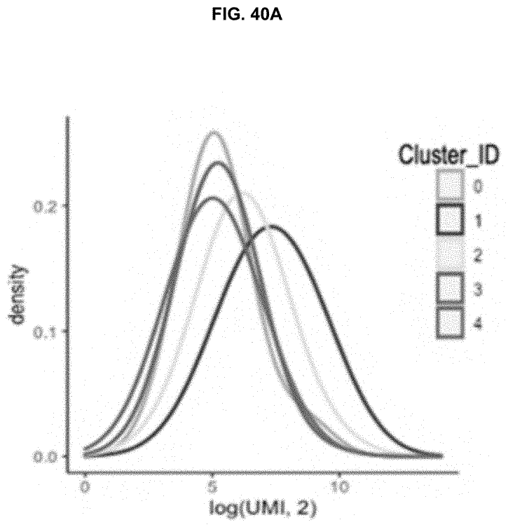

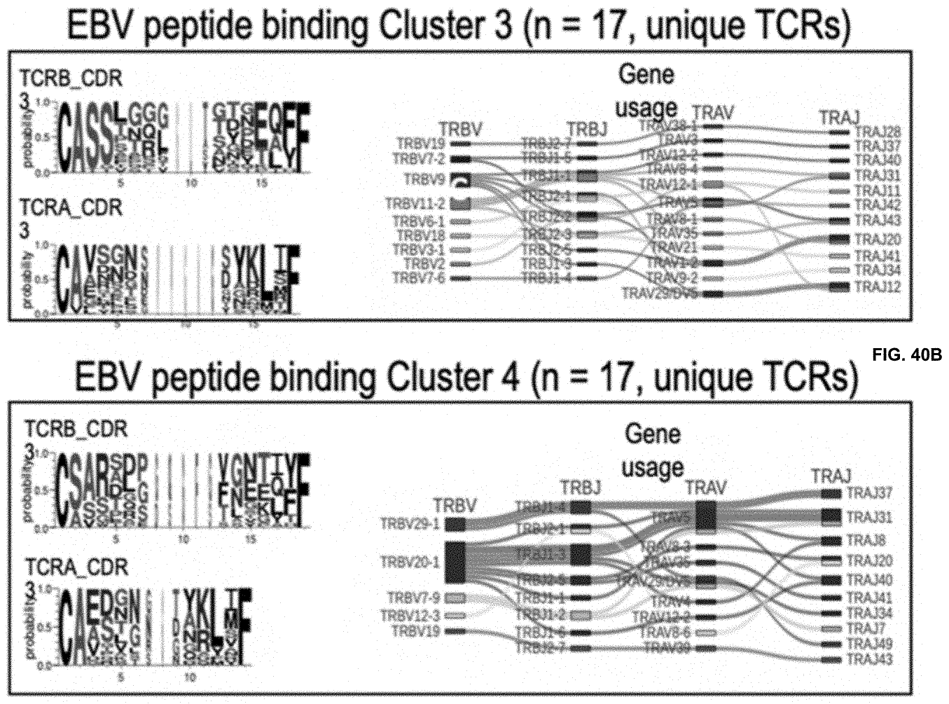

In this study, a Python package TCRAI was developed, with which the robustness of deep-learning classifiers in predicting TCR-pMHC specific bindings is demonstrated. Due to the importance of the CDR3 region in determining the specificity of TCRs to a given antigen, it is tempting to build a predictive model harnessing only this information, as others have. However, due to highly conserved gene usage for many pMHCs, the VJ gene usage is found to be an important predictive element of TCRAI, particularly in the case of few unique pMHC binding TCRs in the dataset. The predictive performance of models that receive CDR3 information outperform gene-level only models in the case where there are more than at least on the order of 100 pMHC binding TCRs was observed (FIG. 39), indicating that one requires this volume of data for these models to be able to extract useful sequence motifs from the CDR3.

It has been shown that TCRAI can not only perform state-of-the-art classification of TCR-pMHC specific binding but can also identify groups of TCRs with differing binding profiles. Partnering the dextramer UMI counts with TCR sequence information allowed for the investigation of differing binding abilities between these groups. The findings indicate that as the volume of high-throughput TCR pMHC binding data grows, so will the ability to discover new TCR motifs and pair these with not only UMI, but also wider multi-Omics data. The ability to investigate, for example, different transcriptional regulation of T cell receptor signaling between groups of TCRs with different binding mechanisms would be very exciting not only for broad scientific questions, but also for the development of T cell therapeutics.

T cell antigen specific recognition can potentially be studied virtually (versus experimentally) using TCRAI. Immune monitoring of T cell antigen specific recognition has been applied to determine the immune responses against specific antigens (e.g. SARS-COV2, tumor-specific antigens and peptide vaccines) and their possible correlation with disease severity, clinical outcome in patients receiving immunotherapies. However, experimentally mapping TCR sequences to antigen specificity is costly and labor intensive. With adequate training data for a particular pMHC, the TCRAI classifier presented here can assign probabilities of pMHC binding to each TCR sequence of interest without conducting binding assays. In this study, the multinomial prediction mode of this classifier has been validated (FIG. 35), meaning it could be used for selecting highly specific TCRs for safe T cell related therapies.

The ability to assess biologically relevant T cell reactivities is important for the interrogation and monitoring of immune responses to pathogens and other disease states. Most of the T cell reactivities recovered (94%) were matched with the appropriate HLA type/supertype, and further, that the phenotypes of multimer positive cells were largely restricted to memory T cell compartments, indicating that relevant memory reactivities from prior functional T cell responses are resolvable with this technology. Paired αβ TCR sequencing revealed multiple TCR sequences that were specific for individual multimers, reinforcing the broad antigenic immune responses to common viral challenges.

While a low degree of HLA mismatched reactivities were recovered, these were significantly enriched in non-expanded naïve T cells relative to memory subsets, potentially revealing antigen-specific interactions to previously unexposed targets or those that did not culminate in functional T cell responses. Additionally, a range of TCR avidities could be recovered in these experiments, which can contribute to the detection of unexpected binding patterns. Dextramers are highly multimerized and likely to detect a broader range of TCR binding avidity than traditional tetramer reagents. Furthermore, a range of fluorescent dextramer intensities were sorted in the multimer-positive gating, so even low-frequency, lower-avidity TCR interactions were captured in this highly sensitive single cell assay.

3. Materials and Methods

i. The 10× Genomics Single Cell Immune Profiling Datasets

10× Genomics data used for this study were downloaded from: support.10×genomics.com/single-cell-vdj/datasets

ii. Identification of pMHC Binding T Cell Phenotypes

Seurat V3 single-cell sequencing analysis R package was used for the classification analysis based on single cell RNA-seq data. Since the significant enrichment of TCR VJ gene usages was observed in identified pMHC binding T cells, the TCR genes were taken out from the classification. So, cell clusters will not be dominated by their shared VJ gene usage. Then, all other gene expression of identified binding T cells was normalized and scaled using Seurat V3 default parameters. PCA was run on normalized and transformed UMI counts on variably expressed genes. Top 10 PCs were used for the cell classification. UMAP was used for classification visualization.

iii. Curation of Reported pMHC Specific Binding Paired TCRs

Raw files were downloaded from VDJdb(42) (vdjdb.cdr3.net/) and The Pathology-associated TCR database (friedmanlab.weizmann.ac.il/McPAS-TCR/). The data was processed to get pMHC TCR binding following the following criteria: for VDJdb, paired α- or β-chain CDR3 amino acid sequences were required for each “complex.id”; TCRs annotated with “source” from 10× genomics were removed; “Species”=“Human” was filtered for. For McPAS-TCR, known “Epitope.ID” were required in the full data and having “CDR3.alpha.aa” and “CDR3.beta.aa”; Similarly, for VDJdb, were filtered for Human TCRs.

iv. Normalization of High-Throughput TCR-pMHC Binding Data

ICON, an Integrative COntext-specific Normalization method, was developed to identify reliable TCR-pMHC interactions. It takes multi-omics single cell sequencing data generated from a multiplexed multimer binding platform, like 10× Genomics Immune Map as input data, including single cell RNA-seq, paired αβ chain single cell TCR-seq, dCODE-Dextramer-seq and cell surface protein expression sequencing—also named CITE-seq. ICON includes the following major steps (FIG. 25A and FIG. 26):

Step 1: Single Cell RNA-Seq Based Filtering of Low-Quality Cells

It filters out low quality cells such as doublets and dead cells. The T cells with an unexpectedly high number of genes (e.g. >2500 genes per cell) were categorized as doublets and cells with a high fraction of mitochondrial gene expression (e.g. ratio of mitochondrial gene expression to the total gene expression >0.2) or too few genes detected (<200 genes per cell) were classified as dead cells (FIG. 26A).

Step 2: Single Cell dCODE-Dextramer-Seq Based Background Estimation

Six negative control dextramers were designed for estimating the background noise from the multiplexed dextramer binding assay. To inspect signal and noise distributions, the maximum dextramer signals in UMI (Unique Molecular Identifier) of negative control dextramers and test dextramers for each cell were used to represent the worst noise and best dextramer binding of each T cell. The density distributions of these two types of dextramer signals are shown in FIG. 26B. The background cutoffs (grey dash lines in FIG. 26B) were empirically chosen for each donor.

Step 3: Selecting T Cells with Paired αβ Chains Based on Single Cell TCR-Seq

T cells that have only a single chain were removed. For T cells with multiple a- or β-chains detected, the ones with highest UMI counts were assigned to each T cell.

Step 4: Dextramer Signal Correction

Each dextramer has its own optimal binding condition, however it is impossible to arrange the experimental conditions such that a multiplexed dextramer binding assay is optimal for every dextramer. This results in multiple dextramers binding to the same T cell/clone, as observed in this high throughput dataset (FIG. 26C). To correct for this effect, dextramer signals were penalized if simultaneously binding to the same T cell/clone, using the following technique.

Defining the background noise subtracted dextramer signal for the i^{th} T cell binding the j^{th} dextramer as Eᵢⱼ, the fraction of dextramer signal due to binding of the j^{th} dextramer for the i^{th} T cell is further denoted as:

$${RC}_{ij} = \frac{{E}_{ij}}{{\sum }_{{j}^{′} = 1}^{n} {E}_{{ij}^{′}}} . \qquad ( 1 )$$

Denoting the TCR clonotype of the i^{th} T cell as kᵢ, and the number of T cells belonging to clonotype kᵢ that bind dextramer j as T_(kᵢⱼ), the fraction of T cells that belong to clonotype kᵢ that bind the i^{th} dextramer is denoted as:

$${RT}_{{k}_{i} j} = \frac{{T}_{{k}_{i} j}}{{\sum }_{{j}^{′} = 1}^{n} {T}_{{k}_{i} {j}^{′}}} . \qquad ( 2 )$$

Using these quantities, the corrected dextramer signal is calculated for the i^{th} T cell binding the j^{th} dextramer as:

Sᵢⱼ=Eᵢⱼ(RCᵢⱼ)²RTₖⱼ

Step 5: Cell- and pMHC-Wise Dextramer Signal Normalization and Binder Identification

To make all the dextramer binding signals comparable, the corrected dextramer binding signals were log-ratio normalized across 44 testing dextramers within a cell. pMHC-wise normalization was subsequently conducted based on Log-Rank distribution. Normalized dextramer UMI>0 was empirically chosen as the cutoff for pMHC specific binders.

v. Regeneron Oligo-Tagged Dextramer Staining and Sorting

CD8+ T cells were enriched from healthy donor PBMC using Miltenyi CD8+ T cell negative enrichment (Mitenyi). The cells were then incubated for 45 minutes with benzonase (Millipore) and dasatinib (Axon) before being stained with oligo-tagged dextramer pools (Immudex, see Table 2) for 30 minutes at room temperature. Cells were then stained with fluorescently labeled for CD3 (BD Biosciences, cat #612750), CD4 (BD Biosciences, cat #563919, CD8 (BD Biosciences, cat #612889), CCR7 (Biolegend, cat #353218), and CD45RA (Biolegend, cat #304238) and CITE-seq antibodies for an additional 30 minutes on ice. Utilizing an Astrios cell sorter (Beckman Coulter), fluorescence activated cell sorting (FACS) gating on forward scatter plot, side scatter plot, and fluorescent channels was set to select live cells while excluding debris and doublets. A 100 μm nozzle was used to sort single CD3+CD8+dextramer+ cells for further processing.

vi. Building a Neural Network Based Classifier TCRAI

Though TCRAI provides a flexible framework for the design of TCR classifiers, a specific and consistent architecture was used throughout this work, which is described in detail below. Aside from its flexible architecture, some key differences from the DeepTCR architecture are the use of 1D convolutions and batch normalization for the CDR3 sequences, and lower dimensional representations for the genes. These changes give improved model regularization and force the model to learn stronger gene associations.

In order to process the input information of the TCR into numerical format the following method was applied. For each CDR3 sequence, amino acids are first converted to integers, and subsequently these integer vectors are encoded into a one-hot representation. For the V and J genes a dictionary of gene type to integer is separately built for each V and J gene and use these to convert each gene to an integer.

The neural network architecture applied to the processed input information includes embedding layers, and convolutional networks. Specifically, processed CDR3 residues were embedded into a 16-dimensional space via a learned embedding, and the resulting numeric CDR3s are fed through 3 1D convolutional layers, with filters of dimensions, kernel widths and strides. Each convolution is activated by an exponential linear unit activation and is followed by dropout and batch normalization. Following these three convolutional blocks, global max pooling is applied to the final features, this process encodes each CDR3 by a vector of length 256, a “CDR3 fingerprint”. The processed gene input for each gene is one-hot encoded and embedded into a reduced dimensional space (16 for V genes, and 8 for J genes) via a learned embedding, giving a “fingerprint” of each gene as a vector. The fingerprints of all selected CDR3s and genes are concatenated together into a single vector, the “TCRAI fingerprint.” The TCRAI fingerprint is passed through one final full-connected layer to give binomial predictions (single output value, sigmoid activation), regression predictions (single output, no activation), or multinomial predictions (multiple output values, softmax activation). Binomial and multinomial predictions are focused on in this work.

TCR sequencing files were collected as a raw csv formatted file from 10× Genomics. Sequencing files were parsed to take the amino acid sequence of the CDR3 after removing unproductive sequences. Clones with different nucleotide sequences but the same matched amino acid sequence from CDR3s and the V, D, J genes were aggregated together under one TCR. Thus, each TCR record used here includes single paired α and β TCR chains, with CDR3 amino acid sequence and V, J genes for each chain.

The data is split into training (76.5%), validation (13.5%), left-out test set (10%) for each model, and subsequently a 5-fold Monte-Carlo cross-validation (MCCV) is performed on the training set. The model is trained by minimizing the cross-entropy loss via the Adam optimizer, and the cross-entropy loss is weighted by weights 1/(number of classes*fraction of samples in that class) for each class. Early stopping is engaged, via a left-out validation dataset, to prevent overfitting, in which the model ceases training if the validation loss increases for more than 5 epochs and the weights of the model with minimal validation loss are restored. Due to the large number of models being trained here, only the learning rate and batch size are tuned during cross-validation. After cross-validation the optimally performing hyperparameters are chosen and the model is re-trained on the full training set, using the validation set to control early-stopping. The re-trained model is then evaluated on the left-out test set.

vii. TCRAI Fingerprint Analysis

TCRAI models produce both a prediction for a TCR to bind a specific pMHC (or one of many pMHCs, in the multinomial case), and a numerical vector “fingerprint” that describes that TCR within the context of the question of whether it can bind that pMHC. In order to gain an understanding of how the model works, and to identify groups of TCRs with different binding modalities, the distribution of these fingerprints is analyzed. UMAP is used to reduce the fingerprints to a two-dimensional space. When using a model trained on one dataset and inferring fingerprints on another unseen dataset, the UMAP projector is fit with TCRs from the training dataset and the TCRs transformed from the unseen set using that projector.

When clustering TCR fingerprints, the fingerprints of all TCRs of the dataset into two-dimensional space are projected as described above, and then those TCRs that are strong true positives are selected (STPs, binomial prediction >0.95). These STPs are then clustered using a k-means classifier in the two-dimensional space. TCRs from within in each cluster are then collected and used to construct CDR3 motif logos (using weblogo), gene-usage, and UMI distributions by pairing the unique TCR clonotypes within the cluster with all repeated clonotypes in the high throughput data.

viii. DeepTCR Modification

The DeepTCR method was adapted to construct a binary classifier with the adjustments as described below.

For each TCR record the single paired α and β TCR chains were used, with CDR3 amino acid sequence and V, J genes for each chain only, in line with the inputs provided to the TCRAI package. That is, clonality, MHC, or D gene usage was not included to the DeepTCR model. The final output layer was adjusted to give a single binomial output, and hyperparameters of the model were optimized for the problem at hand in the context of the DeepTCR framework.

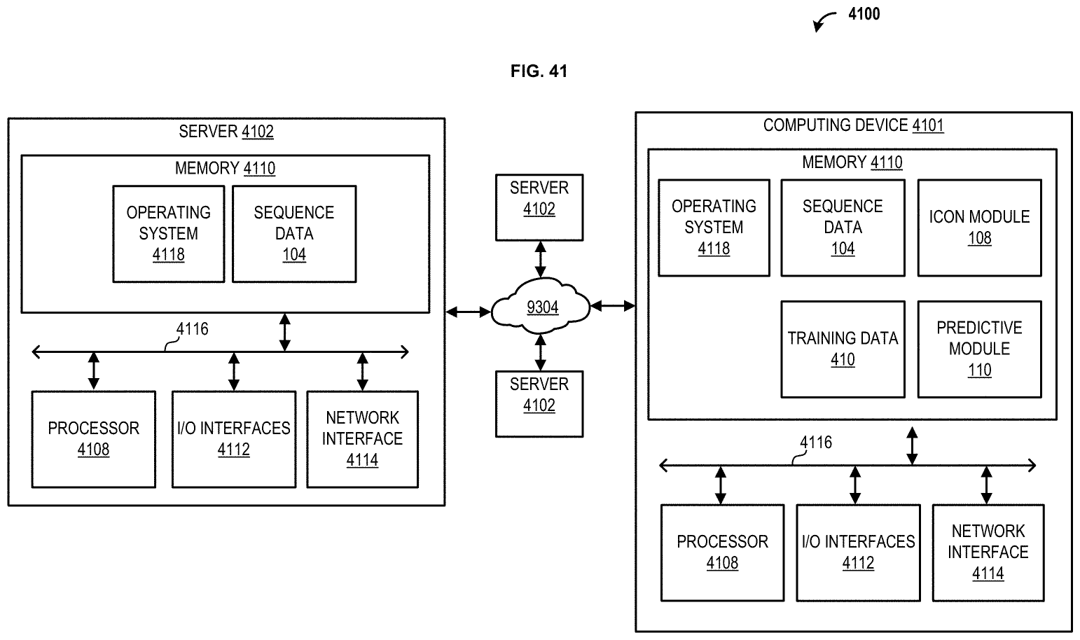

FIG. 41 is a block diagram depicting an environment 4100 comprising non-limiting examples of a computing device 4101 (e.g., the computing device 106) and a server 4102 connected through a network 4104. In an aspect, some or all steps of any described method may be performed on a computing device as described herein. The computing device 4101 can comprise one or multiple computers configured to store one or more of the sequence data 104 (e.g., single cell sequence data, dextramer sequence data, and single cell receptor sequence data), training data 410 (e.g., labeled receptor sequence data), the ICON module 108, the predictive module 110, and the like. The server 1402 can comprise one or multiple computers configured to store the sequence data 104. Multiple servers 4102 can communicate with the computing device 4101 via the through the network 4104. In an embodiment, the server 1402 may comprise a repository for data generated by the single cell immune profiling platform 102.

The computing device 4101 and the server 4102 can be a digital computer that, in terms of hardware architecture, generally includes a processor 4108, memory system 4110, input/output (I/O) interfaces 4112, and network interfaces 4114. These components (4108, 4110, 4112, and 4114) are communicatively coupled via a local interface 4116. The local interface 4116 can be, for example, but not limited to, one or more buses or other wired or wireless connections, as is known in the art. The local interface 4116 can have additional elements, which are omitted for simplicity, such as controllers, buffers (caches), drivers, repeaters, and receivers, to enable communications. Further, the local interface may include address, control, and/or data connections to enable appropriate communications among the aforementioned components.

The processor 4108 can be a hardware device for executing software, particularly that stored in memory system 4110. The processor 4108 can be any custom made or commercially available processor, a central processing unit (CPU), an auxiliary processor among several processors associated with the computing device 4101 and the server 4102, a semiconductor-based microprocessor (in the form of a microchip or chip set), or generally any device for executing software instructions. When the computing device 4101 and/or the server 4102 is in operation, the processor 4108 can be configured to execute software stored within the memory system 4110, to communicate data to and from the memory system 4110, and to generally control operations of the computing device 4101 and the server 4102 pursuant to the software.

The I/O interfaces 4112 can be used to receive user input from, and/or for providing system output to, one or more devices or components. User input can be provided via, for example, a keyboard and/or a mouse. System output can be provided via a display device and a printer (not shown). I/O interfaces 41412 can include, for example, a serial port, a parallel port, a Small Computer System Interface (SCSI), an infrared (IR) interface, a radio frequency (RF) interface, and/or a universal serial bus (USB) interface.

The network interface 4114 can be used to transmit and receive from the computing device 4101 and/or the server 4102 on the network 4104. The network interface 4114 may include, for example, a 10BaseT Ethernet Adaptor, a 100BaseT Ethernet Adaptor, a LAN PHY Ethernet Adaptor, a Token Ring Adaptor, a wireless network adapter (e.g., WiFi, cellular, satellite), or any other suitable network interface device. The network interface 4114 may include address, control, and/or data connections to enable appropriate communications on the network 4104.

The memory system 4110 can include any one or combination of volatile memory elements (e.g., random access memory (RAM, such as DRAM, SRAM, SDRAM, etc.)) and nonvolatile memory elements (e.g., ROM, hard drive, tape, CDROM, DVDROM, etc.). Moreover, the memory system 4110 may incorporate electronic, magnetic, optical, and/or other types of storage media. Note that the memory system 4110 can have a distributed architecture, where various components are situated remote from one another, but can be accessed by the processor 4108.

The software in memory system 4110 may include one or more software programs, each of which comprises an ordered listing of executable instructions for implementing logical functions. In the example of FIG. 41, the software in the memory system 4110 of the computing device 4101 can comprise the sequence data 104, the training data 410, the ICON module 108, the predictive module 110, and a suitable operating system (O/S) 4118. In the example of FIG. 41, the software in the memory system 4110 of the server 4102 can comprise, the sequence data 104, and a suitable operating system (O/S) 4118. The operating system 4118 essentially controls the execution of other computer programs and provides scheduling, input-output control, file and data management, memory management, and communication control and related services.

For purposes of illustration, application programs and other executable program components such as the operating system 4118 are illustrated herein as discrete blocks, although it is recognized that such programs and components can reside at various times in different storage components of the computing device 4101 and/or the server 4102. An implementation of the training module 220 can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” can comprise volatile and non-volatile, removable and non-removable media implemented in any methods or technology for storage of information such as computer readable instructions, data structures, program modules, or other data. Exemplary computer storage media can comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

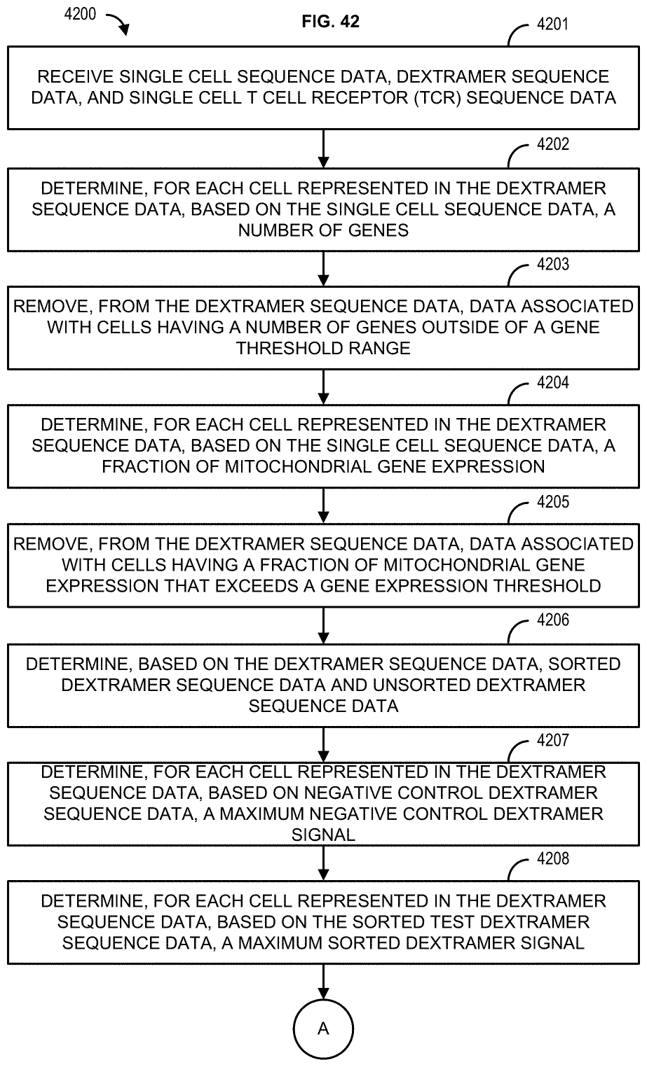

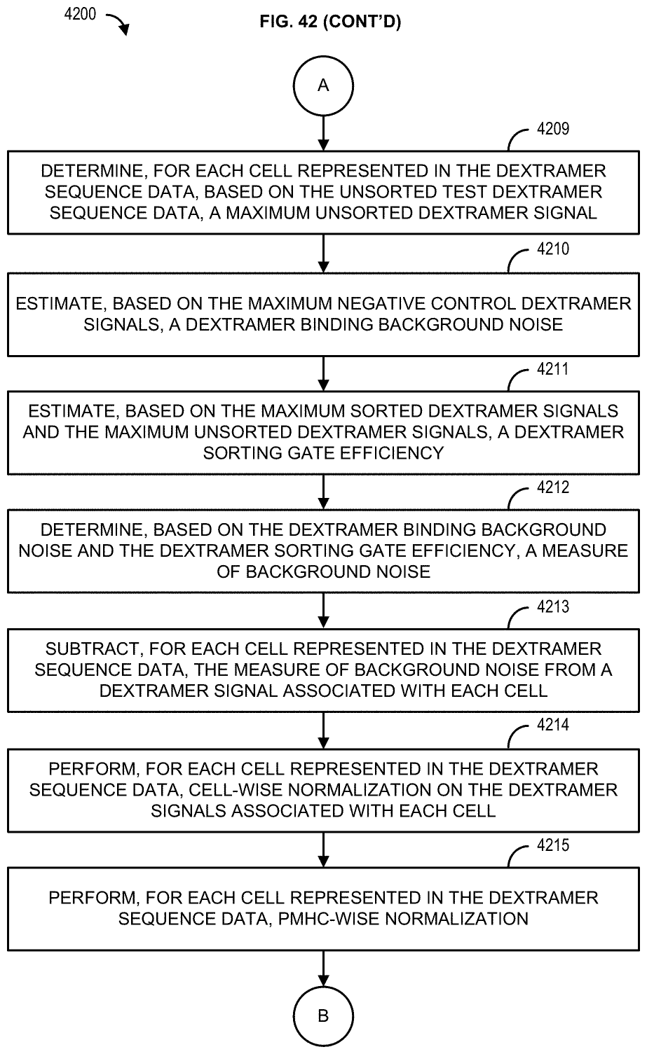

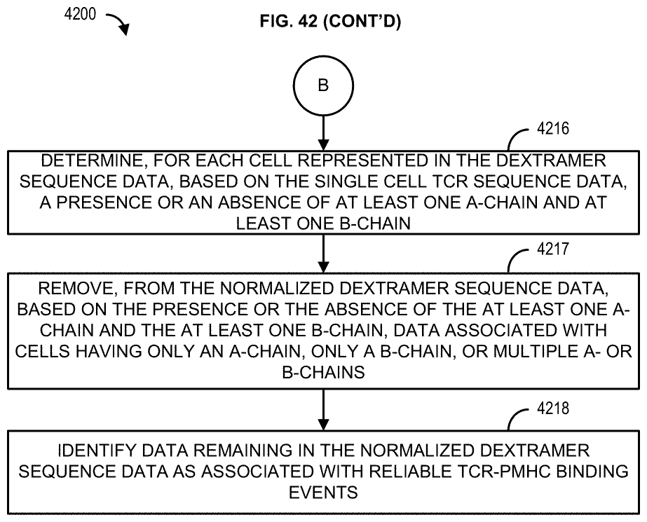

In an embodiment, the ICON module 108 and/or the predictive module 110 may be configured to perform a method 4200, shown in FIG. 42. The method 4200 may be performed in whole or in part by a single computing device, a plurality of electronic devices, and the like. The method 4200 may comprise receiving single cell sequence data, dextramer sequence data, and single cell T Cell Receptor (TCR) sequence data at step 4201. The single cell sequence data may comprise RNA-seq data, the dextramer sequence data may comprise dCODE-Dextamer-seq data, and the single cell T Cell Receptor (TCR) sequence data may comprise TCR-seq data.

The method 4200 may comprise determining, for each cell represented in the dextramer sequence data, based on the single cell sequence data, a number of genes at step 4202.

The method 4200 may comprise removing, from the dextramer sequence data, data associated with cells having a number of genes outside of a gene threshold range at step 4203. By way of example, the gene threshold range may be from about 200 genes to about 2,500 genes.

The method 4200 may comprise determining, for each cell represented in the dextramer sequence data, based on the single cell sequence data, a fraction of mitochondrial gene expression at step 4204.

The method 4200 may comprise removing, from the dextramer sequence data, data associated with cells having a fraction of mitochondrial gene expression that exceeds a gene expression threshold at step 4205. The gene expression threshold can be about 40 percent of total unique molecular identifier counts.

The method 4200 may comprise determining, based on the dextramer sequence data and unsorted dextramer sequence data at step 4206. The sorted dextramer sequence data can comprise sorted test dextramer sequence data and negative control dextramer sequence data. The unsorted dextramer sequence data can comprise unsorted test dextramer sequence data.

The method 4200 may comprise determining, for each cell represented in the dextramer sequence data, based on the negative control dextramer sequence data, a maximum negative control dextramer signal at step 4207. The maximum negative control dextramer signal may be expressed as (Max(nc₁, . . . , ncₙ)), wherein n is a number of negative control dextramers.

The method 4200 may comprise determining, for each cell represented in the dextramer sequence data, based on the sorted test dextramer sequence data, a maximum sorted dextramer signal at step 4208. The maximum sorted dextramer signal may be expressed as (Max(ds₁, . . . , dsₘ)), wherein m is a number of test dextramers.

The method 4200 may comprise determining, for each cell represented in the dextramer sequence data, based on the unsorted test dextramer sequence data, a maximum unsorted dextramer signal at step 4209. The maximum unsorted dextramer signal may be expressed as (Max(du, . . . , duₘ)), wherein m is the number of test dextramers.

The method 4200 may comprise estimating, based on the maximum negative control dextramer signals, a dextramer binding background noise at step 4210. The dextramer binding background noise may comprise determining (P_{99.9}).

The method 4200 may comprise estimating, based on the maximum sorted dextramer signals and the maximum unsorted dextramer signals, a dextramer sorting gate efficiency at step 4211. The dextramer sorting gate efficiency may be expressed as (argmax D_{s, u}). The dextramer sorting gate efficiency may be determined as a maximum difference between (Max (ds₁, . . . , dsₘ)) and (Max(du, . . . , duₘ)).

The method 4200 may comprise determining, based on the dextramer binding background noise and the dextramer sorting gate efficiency a measure of background noise at step 4212. The measure of background noise may be expressed as (d).

The method 4200 may comprise subtracting, for each cell represented in the dextramer sequence data, the measure of background noise from a dextramer signal associated with each cell at step 4213. Subtracting the measure of background noise from a dextramer signal associated with each cell may comprise evaluating (E_{c}=Eₛ−d).

The method 4200 may comprise performing, for each cell represented in the dextramer sequence data, cell-wise normalization on the dextramer signals associated with each cell at step 4214. Performing cell-wise normalization may comprise evaluating:

$${E}_{c}^{′} = \frac{log ( {E}_{c}^{ij} , 10 )}{{\sum }_{j = 1}^{j = N} log ( {E}_{c}^{ij} , 10 )}$$

The method 4200 may comprise performing, for each cell represented in the dextramer sequence data, pMHC-wise normalization at step 4215. Performing pMHC-wise normalization may comprise evaluating:

$${E}^{*} = \frac{{{E}^{′}}_{c}^{ij} - {\overline{{E}^{′}}}_{c}^{j}}{σ ( {E}^{{′}_{c}^{{{ }^{′}}^{′} j}} )}$$

The method 4200 may comprise determining, for each cell represented in the dextramer sequence data, based on the single cell TCR sequence data, a presence or an absence of at least one α-chain and at least one β-chain at step 4216.

The method 4200 may comprise removing, from the normalized dextramer sequence data, based on the presence or the absence of the at least one α-chain and the at least one β-chain, data associated with cells having only an α-chain, only a β-chain, or multiple α- or β-chains at step 4217.

The method 4200 may comprise identifying data remaining in the normalized dextramer sequence data as associated with reliable TCR-pMHC binding events at step 4218.

The method 4200 may further comprise training a predictive model based on the data associated with reliable TCR-pMHC binding events. The method 4200 may further comprise predicting a binding status of a newly presented receptor sequence according to the trained predictive model.

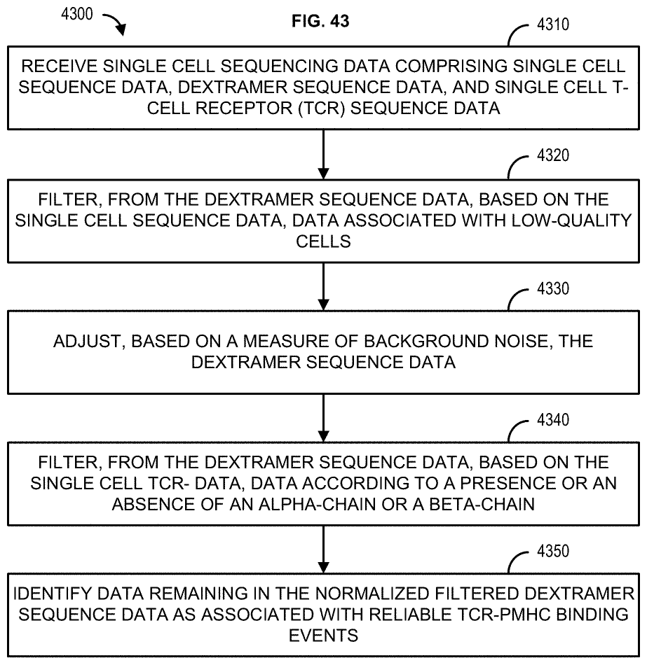

In an embodiment, the ICON module 108 and/or the predictive module 110 may be configured to perform a method 4300, shown in FIG. 43. The method 4300 may be performed in whole or in part by a single computing device, a plurality of electronic devices, and the like. The method 4300 may comprise receiving single cell sequencing data comprising single cell sequence data, dextramer sequence data, and single cell T-Cell Receptor (TCR) sequence data at step 4310. The single cell sequence data may comprise RNA-seq data, the dextramer sequence data may comprise dCODE-Dextamer-seq data, and the single cell T Cell Receptor (TCR) sequence data may comprise TCR-seq data.

The method 4300 may comprise filtering, from the dextramer sequence data, based on the single cell sequence data, data associated with low-quality cells at step 4320. Filtering, from the dextramer sequence data, based on the single cell sequence data, data associated with low-quality cells can comprise determining, for each cell represented in the dextramer sequence data, based on the single cell sequence data, a number of genes, removing, from the dextramer sequence data, data associated with cells having a number of genes outside of a gene threshold range, determining, for each cell represented in the dextramer sequence data, based on the single cell sequence data, a fraction of mitochondrial gene expression, and removing, from the dextramer sequence data, data associated with cells having a fraction of mitochondrial gene expression that exceeds a gene expression threshold. The gene threshold range may be from about 200 genes to about 2,500 genes. The gene expression threshold can be about 40 percent of total unique molecular identifier counts.

The method 4300 may comprise adjusting, based on a measure of background noise, the dextramer sequence data at step 4330. The method 4300 may further comprise determining, based on the dextramer sequence data, sorted dextramer sequence data wherein the sorted dextramer sequence data comprises sorted test dextramer sequence data and negative control dextramer sequence data and unsorted dextramer sequence data, wherein the unsorted dextramer sequence data comprises unsorted test dextramer sequence data. The method 4300 may further comprise determining, for each cell represented in the dextramer sequence data, based on the negative control dextramer sequence data, a maximum negative control dextramer signal, determining, for each cell represented in the dextramer sequence data, based on the sorted test dextramer sequence data, a maximum sorted dextramer signal, and determining, for each cell represented in the dextramer sequence data, based on the unsorted test dextramer sequence data, a maximum unsorted dextramer signal. The maximum negative control dextramer signal may be expressed as (Max(nc₁, . . . , ncₙ)), wherein n is a number of negative control dextramers. The maximum sorted dextramer signal may be expressed as (Max(ds₁, . . . , dsₘ)), wherein m is a number of test dextramers. The maximum unsorted dextramer signal may be expressed as (Max(du, . . . , duₘ)), wherein m is the number of test dextramers.

Adjusting, based on the measure of background noise, the dextramer sequence data can comprise estimating, based on the maximum negative control dextramer signals, a dextramer binding background noise, estimating, based on the maximum sorted dextramer signals and the maximum unsorted dextramer signals, a dextramer sorting gate efficiency, determining, based on the dextramer binding background noise and the dextramer sorting gate efficiency, the measure of background noise (d), and subtracting, for each cell represented in the dextramer sequence data, the measure of background noise from a dextramer signal associated with each cell. The measure of background noise may be expressed as (d). Subtracting the measure of background noise from a dextramer signal associated with each cell may comprise evaluating (E_{c}=Eₛ−d). The method 4300 may further comprise normalizing the dextramer sequence data. Normalizing the dextramer sequence data can comprise performing, for each cell represented in the dextramer sequence data, cell-wise and normalization on the dextramer signals associated with each cell and/or performing, for each cell represented in the dextramer sequence data, pMHC-wise normalization. Performing cell-wise normalization may comprise evaluating:

$${E}_{c}^{′} = \frac{log ( {E}_{c}^{ij} , 10 )}{{\sum }_{j = 1}^{j = N} log ( {E}_{c}^{ij} , 10 )}$$

Performing pMHC-wise normalization may comprise evaluating:

$${E}^{*} = \frac{{{E}^{′}}_{c}^{ij} - {\overline{{E}^{′}}}_{c}^{j}}{σ ( {E}^{{′}_{c}^{{{ }^{′}}^{′} j}} )}$$

The method 4300 may comprise filtering, from the dextramer sequence data, based on the single cell TCR-data, data according to a presence or an absence of an α-chain or a β-chain at step 4340. Filtering, from the dextramer sequence data, based on the single cell TCR-data, data according to the presence or the absence of the α-chain or the β-chain can comprise determining, for each cell represented in the dextramer sequence data, based on the single cell TCR sequence data, a presence or an absence of at least one α-chain and at least one β-chain and removing, from the normalized dextramer sequence data, based on the presence or the absence of the at least one α-chain and the at least one β-chain, data associated with cells having only an α-chain, only a β-chain, or multiple α- or β-chains.

The method 4300 may comprise identifying data remaining in the normalized filtered dextramer sequence data as associated with reliable TCR-pMHC binding events at step 4350.

The method 4300 may further comprise training a predictive model based on the data remaining in the normalized filtered dextramer sequence data. The method 4300 may further comprise predicting a binding status of a newly presented receptor sequence according to the trained predictive model.

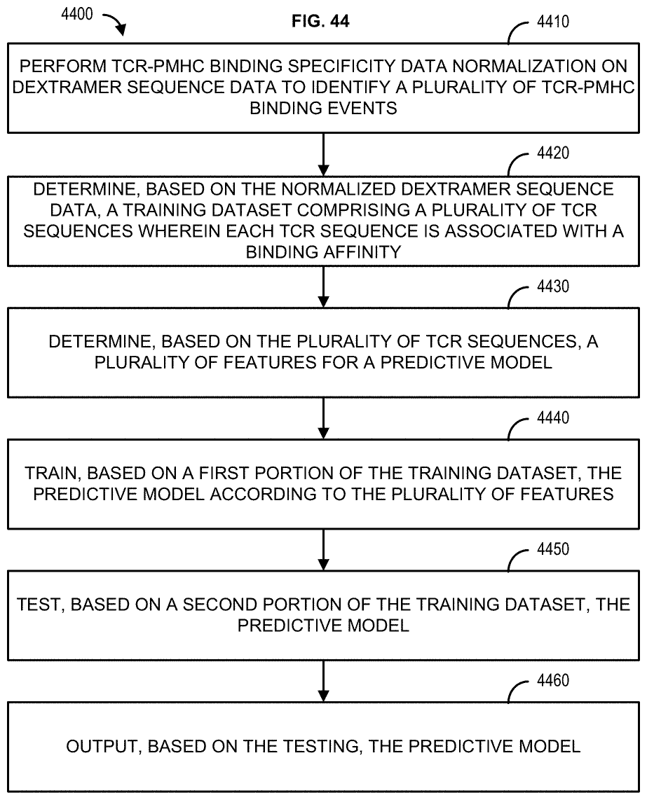

In an embodiment, the ICON module 108 and/or the predictive module 110 may be configured to perform a method 4400, shown in FIG. 44. The method 4400 may be performed in whole or in part by a single computing device, a plurality of electronic devices, and the like. The method 4400 may comprise performing TCR-pMHC binding specificity data normalization on dextramer sequence data to identify a plurality of TCR-pMHC binding events at step 4410. Performing TCR-pMHC binding specificity data normalization on the dextramer sequence data to identify the plurality of TCR-pMHC binding events may comprise some or all of the method 4200 and/or the method 4300.

The method 4400 may comprise determining, based on the normalized dextramer sequence data, a training dataset comprising a plurality of TCR sequences wherein each TCR sequence is associated with a binding affinity at step 4420. Determining, based on the normalized dextramer sequence data, the training dataset comprising the plurality of TCR sequences wherein each TCR sequence is associated with a binding affinity can comprise determining, for each TCR sequence of the plurality of TCR sequences, a paired αβ chain CDR3 amino acid sequence, a V gene identifier, and a J gene identifier and encoding, for each TCR sequence of the plurality of TCR sequences, the paired αβ chain CDR3 amino acid sequence, the V gene segment sequence, and the J gene segment sequence into a one-dimensional input vector. Encoding, for each TCR sequence of the plurality of TCR sequences, the paired αβ chain CDR3 amino acid sequence comprises transforming each alphabetical representation of an amino acid into a numerical representation of the amino acid. Encoding, for each TCR sequence of the plurality of TCR sequences, the V gene identifier and the J gene identifier comprises one hot encoding to generate a categorical and discrete representation of gene names in numerical space.

The method 4400 may further comprise clustering the one-dimensional input vectors into one or more clusters. Clustering the one-dimensional input vectors into one or more clusters comprising applying a KNN clustering algorithm to the one-dimensional input vectors. The one or more clusters are indicative binding strength.

The method 4400 may comprise determining, based on the plurality of TCR sequences, a plurality of features for a predictive model at step 4430. The predictive model can comprise a weighted binary classifier or a Convolutional Neural Network (CNN).

The method 4400 may comprise training, based on a first portion of the training dataset, the predictive model according to the plurality of features at step 4440. Training, based on the first portion of the training dataset, the predictive model according to the plurality of features comprises a training Convolutional Neural Network (CNN). Training, based on a first portion of the training dataset, the predictive model according to the plurality of features comprises applying a class-weighted cost function.

The method 4400 may comprise testing, based on a second portion of the training dataset, the predictive model at step 4450.

The method 4400 may comprise outputting, based on the testing, the predictive model at step 4460.

The method 4400 may further comprise presenting, to the trained predictive model, an unknown TCR sequence and predicting, by the trained predictive model, a binding affinity.

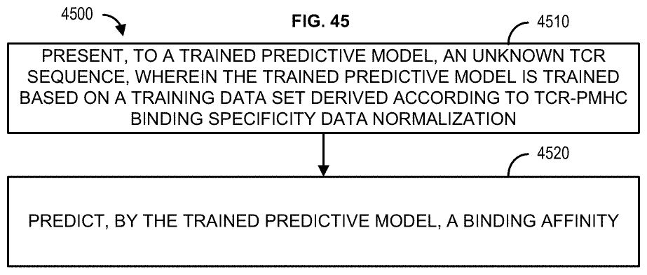

In an embodiment, the ICON module 108 and/or the predictive module 110 may be configured to perform a method 4500, shown in FIG. 45. The method 4500 may be performed in whole or in part by a single computing device, a plurality of electronic devices, and the like. The method 4500 may comprise presenting, to a trained predictive model, an unknown TCR sequence, wherein the trained predictive model is trained based on a training data set derived according to TCR-pMHC binding specificity data normalization at step 4510. The method 4500 may comprise performing the TCR-pMHC binding specificity data normalization on dextramer sequence data to identify a plurality of TCR-pMHC binding events at step 4510. Performing TCR-pMHC binding specificity data normalization on the dextramer sequence data to identify the plurality of TCR-pMHC binding events may comprise some or all of the method 4200 and/or the method 4300.

The method 4500 may comprise predicting, by the trained predictive model, a binding affinity at step 4520. The predictive model can comprise a weighted binary classifier or a Convolutional Neural Network (CNN).

The method 4500 may comprise determining, based on the normalized dextramer sequence data, a training dataset comprising a plurality of TCR sequences wherein each TCR sequence is associated with a binding affinity. The training dataset can comprise a plurality of TCR sequences wherein each TCR sequence is associated with a binding affinity. The training data set can comprise can comprise a paired αβ chain CDR3 amino acid sequence, a V gene identifier, a J gene identifier, and a binding affinity (e.g., yes/no).

The method 4500 may comprise training, based on a first portion of a training dataset, the predictive model according to the plurality of features. Training, based on the first portion of the training dataset, the predictive model according to the plurality of features comprises training a Convolutional Neural Network (CNN). Training, based on the first portion of the training dataset, the predictive model according to the plurality of features comprises training a Convolutional Neural Network (CNN) with a single translationally invariant layer applied to each TCR sequence followed by three fully connected convolutional layers to a final output layer. Training, based on a first portion of the training dataset, the predictive model according to the plurality of features comprises applying a class-weighted cost function. Training, based on the first portion of the training dataset, the predictive model according to the plurality of features comprises training a Neural Network by embedding the one-hot encoded V and J genes of each chain of the TCR sequence via learned embeddings, and concatenating these embeddings together with the output of a Convolutional Neural Network for each CDR3, which is fed the embedded CDR3, forming a 1D numerical vector representing the TCR, followed by passing each numeric TCR sequence through a final fully connected layer.

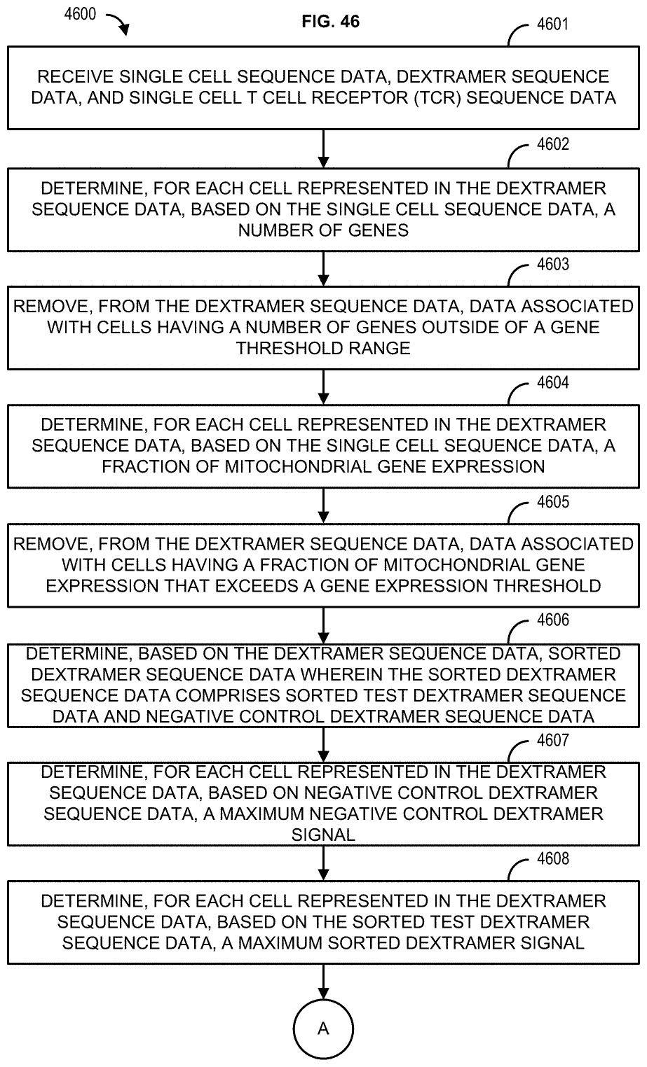

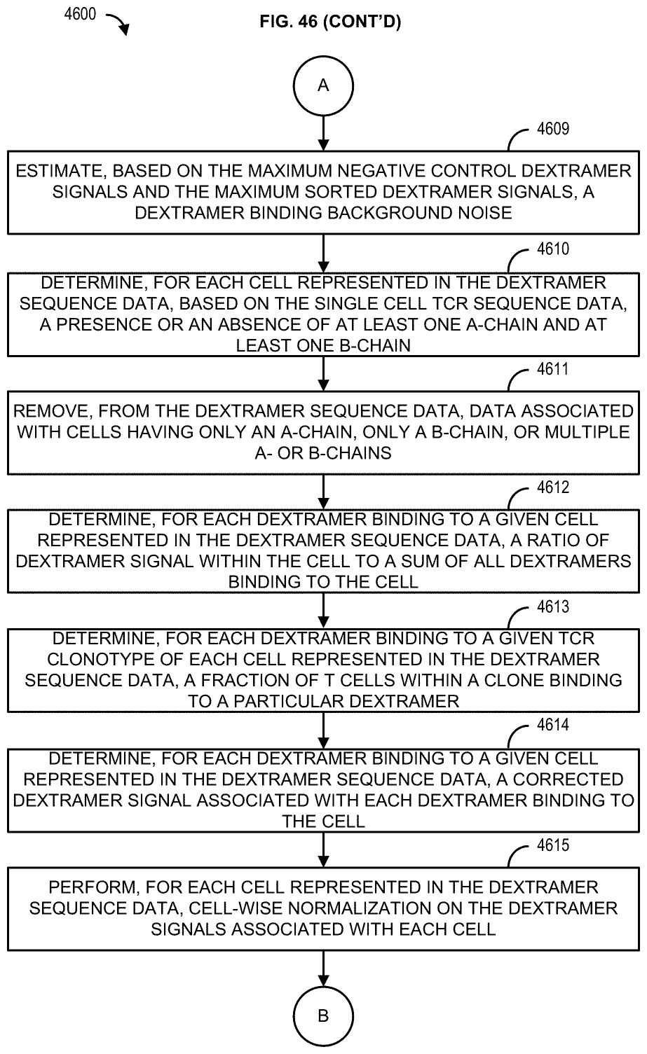

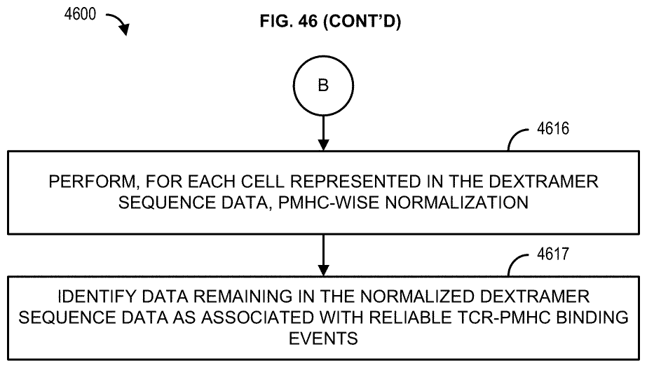

In an embodiment, the ICON module 108 and/or the predictive module 110 may be configured to perform a method 4600, shown in FIG. 46. The method 4600 may be performed in whole or in part by a single computing device, a plurality of electronic devices, and the like. The method 4600 may comprise receiving single cell sequence data, dextramer sequence data, and single cell T Cell Receptor (TCR) sequence data at 4601.

The method 4600 may comprise determining, for each cell represented in the dextramer sequence data, based on the single cell sequence data, a number of genes at 4602.

The method 4600 may comprise removing, from the dextramer sequence data, data associated with cells having a number of genes outside of a gene threshold range at 4603.

The method 4600 may comprise determining, for each cell represented in the dextramer sequence data, based on the single cell sequence data, a fraction of mitochondrial gene expression at 4604.

The method 4600 may comprise removing, from the dextramer sequence data, data associated with cells having a fraction of mitochondrial gene expression that exceeds a gene expression threshold at 4605.

The method 4600 may comprise determining, based on the dextramer sequence data, sorted dextramer sequence data wherein the sorted dextramer sequence data comprises sorted test dextramer sequence data and negative control dextramer sequence data at 4606.

The method 4600 may comprise determining, for each cell represented in the dextramer sequence data, based on the negative control dextramer sequence data, a maximum negative control dextramer signal at 4607.

The method 4600 may comprise determining, for each cell represented in the dextramer sequence data, based on the sorted test dextramer sequence data, a maximum sorted dextramer signal at 4608.

The method 4600 may comprise estimating, based on the maximum negative control dextramer signals and the maximum sorted dextramer signals, a dextramer binding background noise at 4609.

The method 4600 may comprise determining, for each cell represented in the dextramer sequence data, based on the single cell TCR sequence data, a presence or an absence of at least one α-chain and at least one β-chain at 4610.

The method 4600 may comprise removing, from the dextramer sequence data, based on the presence or the absence of the at least one α-chain and the at least one β-chain, data associated with cells having only an α-chain, only a β-chain, or multiple α- or β-chains at 4611.

The method 4600 may comprise determining, for each dextramer binding to a given cell represented in the dextramer sequence data, a ratio of dextramer signal within the cell to a sum of all dextramers binding to the cell (a measure of the dextramer binding specificity to the cell) at 4612. Determining, for each dextramer binding to a given cell represented in the dextramer sequence data, a ratio of dextramer signal within the cell to a sum of all dextramers binding to the cell may comprise determining a background noise subtracted dextramer signal Eᵢⱼ, for the i^{th} T cell binding the j^{th} dextramer and determining a fraction of dextramer signal due to binding of the j^{th} dextramer for the i^{th} T cell by evaluating:

$${RC}_{ij} = \frac{{E}_{ij}}{{\sum }_{{j}^{′} = 1}^{n} {E}_{{ij}^{′}}} .$$

The method 4600 may comprise determining, for each dextramer binding to a given TCR clonotype of each cell represented in the dextramer sequence data, a fraction of T cells within a clone binding to a particular dextramer (a measure of the dextramer binding specificity to the clonotype to which the cell belongs) at 4613. Determining, for each dextramer binding to a given TCR clonotype of each cell represented in the dextramer sequence data, a fraction of T cells within a clone binding to a particular dextramer may comprise determining a TCR clonotype kᵢ, of the i^{th} T cell, determining a number of T cells, Tₖ_{ᵢ}ⱼ, belonging to clonotype kᵢ that bind dextramer j, and determining a fraction of T cells that belong to clonotype kᵢ that bind the j^{th} dextramer by evaluating:

$${RT}_{{k}_{i} j} = \frac{{T}_{{k}_{i} j}}{{\sum }_{{j}^{′} = 1}^{n} {T}_{{k}_{i} {j}^{′}}} .$$

The method 4600 may comprise determining, for each dextramer binding to a given cell represented in the dextramer sequence data, based on the measure the of the dextramer binding specificity to the cell and the measure of the dextramer binding specificity to the clonotype to which the cell belongs, a corrected dextramer signal associated with each dextramer binding to the cell at 4641. Determining, for each dextramer binding to a given cell represented in the dextramer sequence data, based on the measure the of the dextramer binding specificity to the cell and the measure of the dextramer binding specificity to the clonotype to which the cell belongs, a corrected dextramer signal associated with each dextramer binding to the cell may comprise determining the corrected dextramer signal for the i^{th} T cell binding the j^{th} dextramer by evaluating:

Sᵢⱼ=Eᵢⱼ(RCᵢⱼ)²RTₖⱼ.

The method 4600 may comprise performing, for each cell represented in the dextramer sequence data, cell-wise normalization on the dextramer signals associated with each cell;

The method 4600 may comprise performing, for each cell represented in the dextramer sequence data, pMHC-wise normalization at 4615.

The method 4600 may comprise identifying, based on a threshold, data remaining in the normalized dextramer sequence data as associated with reliable TCR-pMHC binding events at 4616.

Those skilled in the art will recognize, or be able to ascertain using no more than routine experimentation, many equivalents to the specific embodiments of the method and compositions described herein. Such equivalents are intended to be encompassed by the following claims.

1.-9. (canceled)

10. A method comprising:
receiving single cell sequencing data comprising single cell sequence data, dextramer sequence data, and single cell T-Cell Receptor (TCR) sequence data;
filtering, from the dextramer sequence data, based on the single cell sequence data, data associated with low-quality cells;
adjusting, based on a measure of background noise, the dextramer sequence data;
filtering, from the dextramer sequence data, based on the single cell TCR-data, data according to a presence or an absence of an α-chain or a β-chain; and
identifying data remaining in the normalized filtered dextramer sequence data as associated with reliable TCR-pMHC binding events.

11. The method of claim 10, wherein filtering, from the dextramer sequence data, based on the single cell sequence data, data associated with low-quality cells comprises:
determining, for each cell represented in the dextramer sequence data, based on the single cell sequence data, a number of genes;
removing, from the dextramer sequence data, data associated with cells having a number of genes outside of a gene threshold range;
determining, for each cell represented in the dextramer sequence data, based on the single cell sequence data, a fraction of mitochondrial gene expression; and
removing, from the dextramer sequence data, data associated with cells having a fraction of mitochondrial gene expression that exceeds a gene expression threshold.

12. (canceled)

13. (canceled)

14. The method of claim 10, further comprising determining, based on the dextramer sequence data, sorted dextramer sequence data wherein the sorted dextramer sequence data comprises sorted test dextramer sequence data and negative control dextramer sequence data and unsorted dextramer sequence data, wherein the unsorted dextramer sequence data comprises unsorted test dextramer sequence data.

15. The method of claim 14, further comprising:
determining, for each cell represented in the dextramer sequence data, based on the negative control dextramer sequence data, a maximum negative control dextramer signal;
determining, for each cell represented in the dextramer sequence data, based on the sorted test dextramer sequence data, a maximum sorted dextramer signal; and
determining, for each cell represented in the dextramer sequence data, based on the unsorted test dextramer sequence data, a maximum unsorted dextramer signal.

16. The method of claim 15, wherein adjusting, based on the measure of background noise, the dextramer sequence data comprises:
estimating, based on the maximum negative control dextramer signals, a dextramer binding background noise;
estimating, based on the maximum sorted dextramer signals and the maximum unsorted dextramer signals, a dextramer sorting gate efficiency;
determining, based on the dextramer binding background noise and the dextramer sorting gate efficiency measure of background noise; and
subtracting, for each cell represented in the dextramer sequence data, the measure of background noise from a dextramer signal associated with each cell.

17. The method of claim 16, wherein estimating, based on the maximum sorted dextramer signals and the maximum unsorted dextramer signals, the dextramer sorting gate efficiency comprises determining a maximum difference between the maximum sorted dextramer signals and the maximum unsorted dextramer signals.

18. (canceled)

19. The method of claim 10, further comprising normalizing the dextramer sequence data, wherein normalizing the dextramer sequence data comprises:
performing, for each cell represented in the dextramer sequence data, cell-wise and normalization on the dextramer signals associated with each cell; and
performing, for each cell represented in the dextramer sequence data, pMHC-wise normalization.

20. The method of claim 10, wherein filtering, from the dextramer sequence data, based on the single cell TCR-data, data according to the presence or the absence of the α-chain or the β-chain comprises:
determining, for each cell represented in the dextramer sequence data, based on the single cell TCR sequence data, a presence or an absence of at least one α-chain and at least one β-chain; and
removing, from the normalized dextramer sequence data, based on the presence or the absence of the at least one α-chain and the at least one β-chain, data associated with cells having only an α-chain, only a β-chain, or multiple α- or β-chains.

21. The method of claim 10, further comprising:
training a predictive model based on the data remaining in the normalized filtered dextramer sequence data;
predicting a binding status of a newly presented receptor sequence according to the trained predictive model;
presenting, to the predictive model, subject TCR sequence data;
determining, by the predictive model, based on the subject TCR sequence data, a subject TCR binding pattern; and
determining, based on a repository of antigen locations and the subject TCR binding pattern, a likelihood that a subject associated with the TCR sequence data has traveled to one or more locations.

22. (canceled)

23. (canceled)

24. (canceled)

25. The method of claim 10, further comprising:
generating, based on the data remaining in the normalized dextramer sequence data associated with reliable TCR-pMHC binding events, a TCR binding pattern for a subject;
receiving, at a subsequent point in time, second single cell sequence data, second dextramer sequence data, and second single cell T Cell Receptor (TCR) sequence data for the subject;
determining, based on the second single cell sequence data, second dextramer sequence data, and second single cell T Cell Receptor (TCR) sequence data for the subject, a second TCR binding pattern; and
identifying, based on a comparison of the TCR binding pattern for the subject and the second TCR binding pattern, the subject.

26. A method comprising:
performing TCR-pMHC binding specificity data normalization on dextramer sequence data to identify a plurality of TCR-pMHC binding events;
determining, based on the normalized dextramer sequence data, a training dataset comprising a plurality of TCR sequences wherein each TCR sequence is associated with a binding affinity;
determining, based on the plurality of TCR sequences, a plurality of features for a predictive model;
training, based on a first portion of the training dataset, the predictive model according to the plurality of features;
testing, based on a second portion of the training dataset, the predictive model; and
outputting, based on the testing, the predictive model.

27. (canceled)

28. The method of claim 26, wherein determining, based on the normalized dextramer sequence data, the training dataset comprising the plurality of TCR sequences wherein each TCR sequence is associated with a binding affinity comprises:
determining, for each TCR sequence of the plurality of TCR sequences, a paired αβ chain CDR3 amino acid sequence, a V gene segment sequence, and a J gene segment sequence; and
encoding, for each TCR sequence of the plurality of TCR sequences, the paired αβ chain CDR3 amino acid sequence, the V gene segment sequence, and the J gene segment sequence into a one-dimensional input vector.

29. The method of claim 28, wherein encoding, for each TCR sequence of the plurality of TCR sequences, the paired αβ chain CDR3 amino acid sequence comprises transforming each alphabetical representation of an amino acid into a numerical representation of the amino acid.

30. The method of claim 28, wherein encoding, for each TCR sequence of the plurality of TCR sequences, the V gene segment sequence and the J gene segment sequence comprises one hot encoding to generate a categorical and discrete representation of gene names in numerical space.

31. (canceled)

32. The method of claim 28, further comprising clustering the one-dimensional input vectors into one or more clusters, wherein clustering the one-dimensional input vectors into one or more clusters comprising applying a KNN clustering algorithm to the one-dimensional input vectors.

33. (canceled)

34. (canceled)

35. The method of claim 26, wherein training, based on the first portion of the training dataset, the predictive model according to the plurality of features comprises training a Neural Network by embedding the one-hot encoded V and J genes of each chain of the TCR sequence via learned embeddings, and concatenating these embeddings together with the output of a Convolutional Neural Network for each CDR3, which is fed the embedded CDR3, forming a 1D numerical vector representing the TCR, followed by passing each numeric TCR sequence through a final fully connected layer.

36. The method of claim 26, wherein training, based on a first portion of the training dataset, the predictive model according to the plurality of features comprises applying a class-weighted cost function.

37. The method of claim 26, further comprising:
presenting, to the trained predictive model, an unknown TCR sequence, wherein trained the predictive model comprises a weighted binary classifier or a Convolutional Neural Network (CNN); and
predicting, by the trained predictive model, a binding affinity.

38. The method of claim 26, further comprising:
presenting, to the predictive model, subject TCR sequence data;
determining, by the predictive model, based on the subject TCR sequence data, a subject TCR binding pattern; and
determining, based on a repository of antigen locations and the subject TCR binding pattern, a likelihood that a subject associated with the TCR sequence data has traveled to one or more locations.

39. (canceled)

40. The method of claim 26, further comprising:
generating, based on the data remaining in the normalized dextramer sequence data associated with reliable TCR-pMHC binding events, a TCR binding pattern for a subject;
receiving, at a subsequent point in time, second single cell sequence data, second dextramer sequence data, and second single cell T Cell Receptor (TCR) sequence data for the subject;
determining, based on the second single cell sequence data, second dextramer sequence data, and second single cell T Cell Receptor (TCR) sequence data for the subject, a second TCR binding pattern; and
identifying, based on a comparison of the TCR binding pattern for the subject and the second TCR binding pattern, the subject.

41.-47. (canceled)
